(19) United States
(12) Patent Application Publication
(10) Pub. No.: US 2025/0382656 A1
(43) Pub. Date: Dec. 18, 2025

(54) CELL-FREE CLOSTRIDIAL NEUROTOXIN ASSAYS

(71) Applicant: IPSEN BIOPHARM LIMITED, Wrexham (GB)

(72) Inventors: Rick BEARDMORE, Wrexham (GB); Sam COWARD, Wrexham (GB); Ciara MATIER, Wrexham (GB); Renske DE LEEUW, Wrexham (GB)

(21) Appl. No.: 19/111,084

(22) PCT Filed: Sep. 14, 2023
(86) PCT No.: PCT/GB2023/052386

§ 371 Date: Mar. 12, 2025

(30) Foreign Application Priority Data
Sep. 14, 2022 (GB) 2213479.5

(51) Int. Cl. C12Q 1/37; A61K 8/66; A61K 38/00; C12N 9/64; G01N 33/543; G01N 33/573

(52) U.S. Cl. CPC C12Q 1/37; A61K 8/66; C12N 9/6489; G01N 33/543; G01N 33/573; A61K 38/00; C12Y 304/24069; G01N 2333/705; G01N 2333/952; G01N 2440/38

(57) ABSTRACT

The present invention is directed to cell-free methods, such as those for determining the clostridial neurotoxin activity of a composition, determining whether or not a composition comprises clostridial neurotoxin polypeptides, and/or determining whether or not clostridial neurotoxin polypeptides or portions thereof comprised in a composition comprise an activity-altering property. The invention is also directed to an isolated capture substrate for a clostridial neurotoxin, use of the same, therapeutic or cosmetic clostridial neurotoxin compositions, and methods for producing the same.

### FIELD OF THE INVENTION

The present invention relates to clostridial neurotoxins and methods for determining activity of the same, in particular.

### BACKGROUND

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, and X (see WO 2018/009903 A2), as well as those produced by C. baratii and C. butyricum. Both tetanus and botulinum toxins act by inhibiting the function of affected neurons, specifically the release of neurotransmitters. While botulinum neurotoxins act at the neuromuscular junction and inhibits cholinergic transmission in the peripheral nervous system, tetanus toxin acts in the central nervous system.

In nature, clostridial neurotoxins (e.g. botulinum neurotoxins [BoNTs]) are synthesised as a single-chain polypeptide that is modified post-translationally by a proteolytic cleavage event to form two polypeptide chains joined together by a disulphide bond. Cleavage occurs at a specific cleavage site, often referred to as the activation site that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin). The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins.

When producing and formulating clostridial neurotoxins for therapeutic and/or cosmetic purposes, there is a need to accurately assess activity of a given composition.

The mouse LD₅₀ assay has historically been the principal assay for the assessment of clostridial neurotoxin activity. The assay simultaneously tests the action of all three domains (i.e. binding, translocation, and protease). In more detail, it defines the median lethal intraperitoneal dose of the toxin at a defined time-point usually 2-4 days after dosing (activity is expressed in mouse LD₅₀ units). Regrettably, however, LD₅₀ assays use large numbers of animals. Moreover, LD₅₀ units are not absolute measurements because they are not biological constants—as such they are highly dependent on the assay conditions. In particular, errors associated with this assay can be as high as 60% between different testing facilities (Sesardic et al. 2003; Biologicals 31(4):265-276).

The mouse flaccid paralysis assay, which is also known as the “mouse abdominal ptosis assay”, relates the activity of clostridial neurotoxins to the degree of abdominal bulging seen after the toxin is subcutaneously injected into the left inguinocrural region of a mouse—the magnitude of the paralysis is dose-dependent. This approach has been proposed as a refinement to the mouse LD₅₀ test, because it relies on a humane endpoint. This assay is approximately 10 times more sensitive than the LD₅₀ assay, uses a sub-lethal dose of toxin and is more rapid than the LD₅₀ test as it provides results in 24 to 48 hours, compared to 72 to 96 hours for a typical LD₅₀ assay. The results from this assay show excellent agreement with the LD₅₀ values (Sesardic et al., 1996; Pharmacol Toxicol, 78 (5): 283-8). Although this assay uses 20% of the animals used in the LD₅₀ assay it still necessitates the use of animals.

Assays such as the mouse/rat phrenic nerve hemi-diaphragm assay (which are based on the use of ex vivo nerve/muscle preparations) relate the activity of a clostridial neurotoxin to a decrease in the amplitude of a twitch response of the preparation after it is applied to a maintenance medium. The usual endpoint of the assay is the time required before a 50% decrease in amplitude is observed. Regrettably, however, the hemi-diaphragm assay (like the LD₅₀ assay) results in the use of large numbers of animals. In addition, the assay requires highly skilled personnel trained in the use of sophisticated and expensive equipment.

All of the above assays have particular failings, notably animal welfare issues. Moreover, none of the above-mentioned assays are suitable for high throughput testing. Thus, there is a need in the art for alternative and/or improved clostridial neurotoxin assays. In addition, when carrying out cell-based assays, clostridial neurotoxins are formulated in cell growth media. Thus, such assays are not amenable to characterising therapeutic and/or cosmetic clostridial neurotoxin formulations, e.g. for identifying optimal excipients and concentrations thereof.

Cell-free assays known in the art typically comprise incubating a test clostridial neurotoxin with a SNARE protein and determining the amount of SNARE protein cleaved by the test clostridial neurotoxin using routine techniques, such as SDS-PAGE and Western blotting. Alternatively, assays are known that test binding of clostridial neurotoxins to cell receptors, such as ELISAs, which employ receptor substrates expressed in prokaryotic host cells (typically E. coli), which lack the post-translational machinery of mammalian cells. Conventional cell-free assays may lack sensitivity to determine small differences in activity. This may be particularly relevant in the context of characterising therapeutic and/or cosmetic clostridial neurotoxin formulations, where differences in activity for formulations with different excipients may be small but therapeutically/cosmetically significant.

The use of clostridial neurotoxins in therapeutic and cosmetic treatment of humans and other mammals is anticipated to expand to an ever-widening range of diseases and ailments that can benefit from the properties of these toxins. In view of this, there is an increasing demand for large-scale manufacture of clostridial neurotoxins and appropriate formulation thereof.

The large-scale manufacture of biotherapeutics, and clostridial neurotoxins in particular, is challenging, with the possibility for unwanted polypeptide modification and/or degradation at multiple stages of the process. Where such unwanted polypeptide modification and/or degradation is present, the activity per pg of clostridial neurotoxin may be significantly reduced compared to a composition lacking said modification and/or degradation. One such unwanted modification is oxidation, which can occur during cellular expression of a clostridial neurotoxin, purification, bioprocessing, formulation, and/or storage. Indeed, oxidation is one of the principal degradation pathways for biotherapeutics. Oxidizing agents such as peroxides, dissolved oxygen, metal ions, light and free-radicals can catalyse oxidation of amino acids, such as methionine, cysteine, histidine, tryptophan, tyrosine, and phenylalanine (Torosantucci et al (2014), Pharm Res, 31, 541-553). In bioprocessing and formulation, metal catalysts may come from metal contaminated buffers and/or metal contact surfaces, with the metal-catalysed oxidation of histidine and methionine residues having been demonstrated to cause loss of activity, for example due to aggregation and/or precipitation of oxidized polypeptides. Moreover, oxidizing agents are typically employed for decontamination in large-scale manufacture. Clostridial neurotoxins are large polypeptides having many surface exposed amino acid residues that are candidates for oxidation.

It is important for therapeutic and/or cosmetic purposes that the amount of unwanted polypeptide modification and/or degradation present in a clostridial neurotoxin composition meets rigorously defined standards. Thus, there is a need to sensitively and/or specifically determine whether a composition comprises such unwanted polypeptide modification and/or degradation. As mentioned above, conventional cell-free assays typically test only one aspect of neurotoxin activity, such as L-chain proteolytic activity or H_{C} domain binding affinity. Therefore, the conventional assays may be of somewhat limited value in this regard, as they do not provide a holistic insight into the nature of the clostridial neurotoxin polypeptides present in a composition. For example, such assays do not allow for the determination of: the amount of contaminating free L-chain or H-chain present in a composition; and/or whether or not the clostridial neurotoxin present in the composition is degraded and/or subject of unwanted modification (e.g. oxidation). In other words, the conventional assays do not provide sufficient information for the skilled person to determine whether any activity observed in a clostridial neurotoxin composition is arising from unwanted polypeptide modification and/or degradation.

The present invention overcomes one or more of the above-mentioned problems.

### SUMMARY OF THE INVENTION

The present inventors have developed a novel cell-free method for determining the clostridial neurotoxin activity of a composition. Advantageously, the cell-free method may be particularly sensitive and/or specific, thereby allowing for the determination of small differences in activity between compositions. This may be particularly advantageous in the context of characterising therapeutic and/or cosmetic clostridial neurotoxin formulations, where differences in activity for formulations with different excipients may be small but therapeutically/cosmetically significant.

Additionally or alternatively, the activity results obtained using a cell-free method of the invention were surprisingly similar to those determined using a cell-based method, while avoiding the disadvantages associated with cell-based methods. In particular, unlike cell-based methods, the methods of the invention are amenable to characterising therapeutic and/or cosmetic clostridial neurotoxin formulations, e.g. for identifying optimal excipients and concentrations thereof, as the clostridial neurotoxins for testing do not need to be formulated in growth media.

Advantageously, the cell-free methods of the invention may also be used to determine whether a composition comprises an activity-altering property (e.g. unwanted polypeptide modification and/or degradation (e.g. oxidation)). For example, the cell-free methods have been shown to accurately predict loss of potency associated with clostridial neurotoxin oxidation.

Moreover, in embodiments where assaying of L-chain activity is carried out in an assay sample comprising dissociated L-chain polypeptides and complexes comprising a capture substrate and clostridial neurotoxin receptor binding domain (H_{C} domain), advantageously, the number of plates used for a given assay can be reduced. Thus, the cleavage assay may be associated with reduced wastage and cost, and more amenable to high-throughput testing.

### DETAILED DESCRIPTION

In one aspect, the invention provides a cell-free method for determining the clostridial neurotoxin activity of a composition comprising clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding a reducing agent for dissociating light-chain (L-chain) polypeptides of the bound clostridial neurotoxin polypeptides; and
(e) determining an amount of cleavable substrates cleaved by the L-chain polypeptides, thereby determining the clostridial neurotoxin activity of the composition.

In one aspect, the invention provides a cell-free method for determining whether or not a composition comprises clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing any unbound clostridial neurotoxin polypeptides;
(d) adding a reducing agent for dissociating any light-chain (L-chain) polypeptides of any bound clostridial neurotoxin polypeptides; and
(e) determining that cleavable substrates have been cleaved, thereby determining that the composition comprises clostridial neurotoxin polypeptides, or determining that cleavable substrates have not been cleaved, thereby determining that the composition does not comprise clostridial neurotoxin polypeptides.

In the foregoing aspect, when a composition does not comprise clostridial neurotoxin polypeptides, there will be no unbound clostridial neurotoxin polypeptides in step (c) and no dissociated L-chain polypeptides in step (d).

In one aspect, the invention provides a cell-free method for determining the clostridial neurotoxin activity of a composition comprising clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding a reducing agent for dissociating light-chain (L-chain) polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining an amount of cleavable substrates cleaved in the assay sample by the L-chain polypeptides, thereby determining the clostridial neurotoxin activity of the composition.

In one aspect, the invention provides a cell-free method for determining whether or not a composition comprises clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing any unbound clostridial neurotoxin polypeptides;
(d) adding a reducing agent for dissociating any light-chain (L-chain) polypeptides of any bound clostridial neurotoxin polypeptides, thereby providing an assay sample comprising any dissociated L-chain polypeptides and any complexes comprising a capture substrate and clostridial neurotoxin receptor binding domain (H_{CC} domain, e.g. H_{C} domain); and
(e) determining that cleavable substrates have been cleaved in the assay sample, thereby determining that the composition comprises clostridial neurotoxin polypeptides, or determining that cleavable substrates have not been cleaved in the assay sample, thereby determining that the composition does not comprise clostridial neurotoxin polypeptides.

In the foregoing aspect, when a composition does not comprise clostridial neurotoxin polypeptides, there will be no unbound clostridial neurotoxin polypeptides in step (c), no dissociated L-chain polypeptides in step (d), and no complexes comprising a capture substrate and clostridial neurotoxin receptor binding domain (H_{CC} domain, e.g. H_{C} domain) in step (d). In other words, when a composition does not comprise clostridial neurotoxin polypeptides, the assay sample may not comprise dissociated L-chain polypeptides and complexes comprising a capture substrate and clostridial neurotoxin receptor binding domain (H_{CC} domain, e.g. H_{C} domain).

In one embodiment, the invention provides a cell-free method for determining that a composition comprises clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding a reducing agent for dissociating light-chain (L-chain) polypeptides of bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining that cleavable substrates have been cleaved in the assay sample, thereby determining that the composition comprises clostridial neurotoxin polypeptides.

The cleavable substrates are separate to the capture substrates (e.g. are not covalently associated therewith, for example are different polypeptides) and may be added before, during, or after (preferably during) a step of adding a reducing agent. Thus, the cleavable substrates are preferably added simultaneously with the reducing agent.

For example, a cell-free method for determining the clostridial neurotoxin activity of a composition comprising clostridial neurotoxin polypeptides may comprise:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding cleavable substrates and a reducing agent for dissociating light-chain (L-chain) polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining an amount of cleavable substrates cleaved in the assay sample by the L-chain polypeptides, thereby determining the clostridial neurotoxin activity of the composition.

The cell-free methods of the present invention are in vitro methods. The term “cell-free” as used herein means that the method is not carried out in a cell or a cell lysate. The components of the invention (e.g. capture substrates and cleavable substrates) are thus preferably prepared recombinantly and isolated from cells. Advantageously, this constitutes a much cleaner system that may be better controlled.

In some embodiments, the components of the invention (e.g. capture substrates and cleavable substrates) have been purified. The term “purified” may be used to refer to a substance such as a polypeptide that is “substantially pure”. Thus, in a composition the components may account for at least 90%, 95%, 99% or 99.9% of total biological material (e.g. fatty acids, nucleic acids, and/or polypeptides) present.

When carrying out a method of the invention, capture substrates may be contacted with a composition under conditions suitable for binding of clostridial neurotoxin polypeptides (when present in the composition) to the capture substrates. For example, 20-500 nM capture substrates may be contacted with 0.1-150 PM clostridial neurotoxin polypeptides. The capture substrates and composition may be contacted for at least 5 minutes, 10 minutes, 30 minutes or 45 minutes, preferably at least 50 minutes. The capture substrates and composition may be contacted for ≤5 hours, ≤4 hours, ≤3 hours, ≤2 hours, or ≤1.5 hours, preferably ≤75 minutes. The capture substrates and composition may be contacted for 5 minutes to 5 hours, 10 minutes to 4 hours, 10 minutes to 3 hours, 30 minutes to 2 hours, or 45 minutes to 75 minutes, preferably 50 minutes to 70 minutes (e.g. 60 minutes). The capture substrates and composition may be incubated at 20-45° C. or 30-40° C. during the contacting, preferably at 35-40° C. (e.g. at 37° C.). Thus, the capture substrates may be contacted for 50-70 minutes and incubated at 35-40° C. during said contacting. It is preferred that the capture substrates and composition are agitated during contacting, e.g. by way of use of a plate shaker. Agitation may be at 100-1000 rpm, 400-800 rpm, or 500-700 rpm, preferably 550-650 rpm (e.g. 600 rpm). In one embodiment, when capture substrates are immobilised on a solid support, any supernatant may be removed prior to contacting the capture substrates with the composition.

The methods herein may be tolerant of non-clostridial neurotoxin components (e.g. buffers and/or excipients) that may be present in the composition. Nevertheless, suitable components may comprise a buffer (e.g. Dulbecco's phosphate-buffered saline (DPBS)), a polypeptide (e.g. bovine serum albumin (BSA), such as 0.5-2% (preferably 1%) BSA), and a detergent (e.g. Tween-20, such as 0.025%-0.1% (preferably 0.05%) Tween-20). Preferably, a composition comprises at least BSA (e.g. 0.5-2% (preferably 1%) BSA). Advantageously, use of BSA may improve sensitivity of the method (FIG. 2), e.g. when compared to casein.

A method may comprise removing clostridial neurotoxin polypeptides that have not bound to the capture substrates, such as clostridial neurotoxin polypeptides that have not bound to the capture substrates via the H_{CC} domain (e.g. H_{C} domain) thereof. The skilled person will appreciate that any step of “removing unbound clostridial neurotoxin polypeptides” may not necessarily remove all unbound clostridial neurotoxin polypeptides. Thus, the method may comprise removing substantially all unbound clostridial neurotoxin polypeptides. The term “substantially all unbound clostridial neurotoxin polypeptides” may mean at least 90%, 95%, 98% or 99% of unbound clostridial neurotoxin polypeptides. Preferably, the method comprises removing 100% of unbound clostridial neurotoxin polypeptides. Removal may be achieved by any suitable technique known in the art. In one embodiment, when capture substrates immobilised on a solid support have been contacted with a composition in accordance with the invention, the supernatant may be removed, thereby removing unbound clostridial neurotoxin polypeptides. Removal may additionally or alternatively comprise a wash step with a suitable buffer, optionally incubated at a suitable temperature and/or under suitable agitation conditions. For example, removing unbound clostridial neurotoxin polypeptides may comprise a wash step with a composition comprising the same non-clostridial neurotoxin components present in the composition contacted with the capture substrates (e.g. a wash buffer). An exemplary composition for the wash step may comprise: a buffer (e.g. Dulbecco's phosphate-buffered saline (DPBS)), a polypeptide (e.g. bovine serum albumin (BSA), such as 0.5-2% (preferably 1%) BSA), and a detergent (e.g. Tween-20, such as 0.025%-0.1% (preferably 0.05%) Tween-20). The wash may be repeated, if necessary. In one embodiment, when capture substrates immobilised on a solid support have been contacted with a composition in accordance with the invention, the supernatant may be removed, and a wash buffer added, the wash buffer may then be removed, thereby removing unbound clostridial neurotoxin polypeptides.

A method may comprise adding a reducing agent for dissociating light-chain (L-chain) polypeptides of any clostridial neurotoxin polypeptides bound to the capture substrates. Any suitable reducing agent may be used, so long as the reducing agent is capable of reducing the di-sulphide bond between the L-chain and H-chain of the clostridial neurotoxin without significantly reducing L-chain activity of the clostridial neurotoxin. A suitable reducing agent may be dithiothreitol (DTT), 2-mercaptoethanol, or tris(2-carboxyethyl) phosphine (TCEP). The reducing agent may be present at any suitable concentration, however, exemplary concentrations of DTT include 2.5-7.5 mM, preferably 5 mM. The reducing agent may be contacted with any clostridial neurotoxin polypeptides bound to the capture substrates for at least 30 minutes, 60 minutes or 120 minutes, preferably at least 160 minutes. The reducing agent may be contacted with any clostridial neurotoxin polypeptides bound to the capture substrates for ≤6 hours, ≤5 hours, or ≤4 hours, preferably ≤3 hours. The reducing agent may be contacted with any clostridial neurotoxin polypeptides bound to the capture substrates for 30 minutes to 6 hours, 1 hour to 5 hours, or 2 hours to 5 hours, preferably 2.5 hours to 3.5 hours (e.g. 3 hours). The reducing agent and any clostridial neurotoxin polypeptides bound to the capture substrates may be incubated during the contacting at 20-45° C. or 30-40° C. during the contacting, preferably at 35-40° C. (e.g. at 37° C.). Thus, the reducing agent may be contacted with any clostridial neurotoxin polypeptides bound to the capture substrates for 2.5-3.5 hours and incubated at 35-40° C. during said contacting. It is preferred that the reducing agent and any clostridial neurotoxin polypeptides bound to the capture substrates are agitated during contacting, e.g. by way of use of a plate shaker. Agitation may be at 100-1000 rpm, 400-800 rpm, or 500-700 rpm, preferably 550-650 rpm (e.g. 600 rpm).

A method may comprise a step of separating L-chain polypeptides from the capture substrate and clostridial neurotoxin H_{CC} domain (e.g. H_{C} domain) complexes (e.g. the complexes comprising the capture substrate and clostridial neurotoxin H_{CC} domain (e.g. H_{C} domain)) following contacting with the reducing agent. In one embodiment, when the capture substrates are immobilised on a solid support, the supernatant comprising the L-chain polypeptides may be placed in a separate container (e.g. a vial or well). The L-chain polypeptides may then be contacted with a cleavable substrate.

However, it is most preferred that the dissociated L-chain polypeptides are not separated from the capture substrate and clostridial neurotoxin H_{CC} domain (e.g. H_{C} domain) complexes (e.g. the complexes comprising the capture substrate and clostridial neurotoxin H_{CC} domain (e.g. H_{C} domain)) following contacting with the reducing agent (e.g. and are not transferred to a separate container, such as vial or well). In other words, while not being part of the complexes, the L-chain polypeptides may be maintained in a solution comprising the complexes (e.g. the complexes may be immobilised). Advantageously, the present inventors have found that this step substantially improves the sensitivity of the cell-free assay. This may be especially the case when the cleavable substrates comprise a first luciferase domain, a linker comprising a clostridial neurotoxin cleavage site, and a second luciferase domain, as described herein. Prior to this finding, it was expected that the non-specific background binding would have been substantial and that specificity of the method would have been negatively affected. Unexpectedly, this was not the case and it was found that adopting this method not only significantly improved the sensitivity of the assay, but did so without substantially increasing non-specific background, as detailed in the present Examples. In view of this finding, advantageously, the number of plates used for a given assay can also be reduced. Thus, such methods may be associated with reduced wastage and cost, and be more amenable to high-throughput testing. In such embodiments (which are most preferred), activity of the L-chain is assessed in the presence of the capture substrate and clostridial neurotoxin H_{CC} domain (e.g. H_{C} domain) complexes (e.g. the complexes comprising the capture substrate and clostridial neurotoxin H_{CC} domain (e.g. H_{C} domain)). The combination comprising the dissociated L-chain polypeptides, capture substrate and clostridial neurotoxin H_{CC} domain (e.g. H_{C} domain) complexes (e.g. as described herein, e.g. the complexes comprising the capture substrate and clostridial neurotoxin H_{CC} domain (e.g. H_{C} domain)) may be referred to herein as an “assay sample”. The cleavable substrates may be added to the assay sample after the step of adding a reducing agent for dissociating light-chain (L-chain) polypeptides. Although, preferably, the cleavable substrates are added at the same time as the reducing agent (e.g. simultaneously with the reducing agent). Thus preferably, an assay sample is a combination comprising the dissociated L-chain polypeptides, capture substrate and clostridial neurotoxin H_{CC} domain (e.g. H_{C} domain) complexes, and cleavable substrates. The cleavable substrates may be present in the assay sample for at least 30 minutes, 60 minutes or 120 minutes, preferably at least 160 minutes. The cleavable substrates may be present in the assay sample for ≤6 hours, ≤5 hours, or ≤4 hours, preferably ≤3 hours. The cleavable substrates may be present in the assay sample for 30 minutes to 6 hours, 1 hour to 5 hours, or 2 hours to 5 hours, preferably 2.5 hours to 3.5 hours (e.g. 3 hours). The assay sample comprising the cleavable substrates may be incubated at 20-45° C. or 30-40° C. during the contacting, preferably at 35-40° C. (e.g. at 37° C.).

Thus, cleavable substrates may be present in the assay sample for 2.5-3.5 hours and the assay sample comprising the cleavable substrates incubated at 35-40° C. during said contacting. It is preferred that the assay sample comprising the cleavable substrates is agitated during contacting, e.g. by way of use of a plate shaker. Agitation may be at 100-1000 rpm, 400-800 rpm, or 500-700 rpm, preferably 550-650 rpm (e.g. 600 rpm).

Methods in which the L-chain polypeptides are not separated from the capture substrate and clostridial neurotoxin H_{CC} domain (e.g. H_{C} domain) complex following contacting with the reducing agent may be more practical and easier to perform and/or may be associated with reduced variability (assay noise). Advantageously, the improved sensitivity (e.g. as measured by the EC₅₀ value) may enable the testing of compositions comprising very low amounts of clostridial neurotoxin polypeptides. Such compositions may be drug product compositions produced by diluting a drug substance (obtainable after purification of clostridial neurotoxin polypeptides). Any such advantages may be evident by comparison with an equivalent method in which L-chain polypeptides are separated from the capture substrate and clostridial neurotoxin H_{CC} domain (e.g. H_{C} domain) complexes following contacting with the reducing agent (e.g. and transferred to a separate vial/well).

A capture substrate described herein (e.g. in the context of a method, capture substrate use or isolated capture substrate of the invention) may be part of a complex comprising the capture substrate and a clostridial neurotoxin receptor binding domain (H_{CC} domain or H_{C} domain). Said complexing may occur following a contacting step described herein. The complex may comprise the capture substrate and a full-length clostridial neurotoxin polypeptide comprising or consisting of the light-chain and heavy-chain thereof. The complex preferably comprises the capture substrate and the heavy-chain of the clostridial neurotoxin (e.g. comprising or consisting of the translocation domain [H_{N} domain] and H_{C} domain). Said complex preferably lacks the light-chain of the clostridial neurotoxin when a reducing agent has been added to a complex comprising a capture substrate and a clostridial neurotoxin polypeptide. In said complexes the H_{C} domain or H_{CC} domain of the heavy-chain is preferably bound to the capture substrate (e.g. by way of non-covalent interactions).

Thus, an assay sample described herein preferably comprises dissociated L-chain polypeptides (e.g. where present) and complexes comprising a capture substrate and the heavy-chain of a clostridial neurotoxin. Said heavy chain preferably comprises or consists of the translocation domain (H_{N} domain) and H_{C} domain of the clostridial neurotoxin.

Cleavable substrates may be used in a method of the invention (e.g. present in an assay sample) at a concentration of 1-1000 nM, such as 10-500 nM, preferably at 50-150 nM (e.g. 100 nM).

Cleavable substrates are preferably not directly or indirectly immobilised on a solid support (e.g. on a plate, such as in a well thereof). By avoiding such immobilisation (e.g. by providing the cleavable substrates free in solution), sensitivity may be increased. In other words, sensitivity may be decreased by directly or indirectly immobilising cleavable substrates on a solid support (e.g. on a plate, such as in a well thereof). Said decreased sensitivity may occur by reducing the total number of available binding sites on the solid support (e.g. on a plate, such as in a well thereof).

A capture substrate may be any substrate capable of binding to a clostridial neurotoxin. Suitably, the capture substrate for use in a method of the invention is selected for the clostridial neurotoxin and/or clostridial neurotoxin H_{CC} domain (e.g. H_{CC} domain) being assayed. A capture substrate may comprise a clostridial neurotoxin receptor polypeptide or a ganglioside to which a clostridial neurotoxin binds. The method of the invention may employ the use of a combination of a capture substrate comprising a clostridial neurotoxin receptor polypeptide and a capture substrate comprising a ganglioside. The capture substrate comprising a clostridial neurotoxin receptor polypeptide and a capture substrate comprising a ganglioside may be complexed (e.g. via non-covalent interactions). Such complexing may occur before, during, or after contact with and/or binding of a clostridial neurotoxin polypeptide or part (e.g. an H_{C} or H_{CC} domain) thereof. In some instances, the capture substrate comprising a clostridial neurotoxin receptor polypeptide may be directly or indirectly immobilised on a solid support while the capture substrate comprising a ganglioside may be added thereto. The capture substrate comprising a ganglioside may then form a complex with the immobilised capture substrate comprising a clostridial neurotoxin receptor polypeptide. However, it is preferred that a capture substrate comprises a clostridial neurotoxin receptor polypeptide. Thus, in some embodiments, it is preferred that the method does not comprise the use of a capture substrate comprising (or consisting of) a ganglioside (e.g. GT1b). In fact, combined use of a capture substrate comprising a clostridial neurotoxin receptor polypeptide (e.g. comprising an extracellular portion of a neuronal clostridial neurotoxin receptor polypeptide) and a capture substrate comprising a ganglioside was shown to reduce sensitivity of the method, e.g. when the clostridial neurotoxin receptor polypeptide (e.g. the extracellular portion of a neuronal clostridial neurotoxin receptor polypeptide) used was SV2c and the ganglioside was GT1b. In contrast, combined use of a capture substrate comprising a clostridial neurotoxin receptor polypeptide (e.g. comprising an extracellular portion of a neuronal clostridial neurotoxin receptor polypeptide) and a capture substrate comprising a ganglioside was shown to increase sensitivity of the method when the clostridial neurotoxin receptor polypeptide (e.g. the extracellular portion of a neuronal clostridial neurotoxin receptor polypeptide) used was SYT-I. Thus, preferably, when the capture substrate comprises SYT-I (e.g. an extracellular portion thereof), capture substrates comprising a ganglioside (preferably GT1b) may also be used. Said capture substrates may be particularly advantageous when the clostridial neurotoxin comprises a modified BoNT/B H_{CC} domain as described herein.

In some instances herein, “capture substrates” are referred to. However, this is to indicate that more than one capture substrate molecule is present when carrying out the method. It is not intended to necessarily indicate that two or more different types of capture substrates are present, although this is encompassed. Thus, the capture substrates may be of one type (e.g. all capture substrates comprise a receptor polypeptide or a ganglioside for a first clostridial neurotoxin) or multiple types (e.g. a portion of capture substrates comprise a receptor polypeptide for a first clostridial neurotoxin type and a portion of capture substrates comprise a receptor polypeptide for a second, different clostridial neurotoxin). Preferably, the capture substrates are of one type.

Gangliosides are oligoglycosylceramides derived from lactosylceramide and containing a sialic acid residue such as N-acetylneuraminic acid (‘NANA’ or ‘SA’ or ‘Neu5Ac’ or ‘NeuAc’). In some embodiments, the sialic acid component is N-glycolyl-neuraminic acid (Neu5Gc), or a Neu5Ac analogue in which the amine group is replaced by OH(3-deoxy-D-glycero-D-galacto-nonulosonic acid, given the abbreviation ‘KDN’). Gangliosides are defined by a nomenclature system proposed by Svennerholm in which M, D, T and Q refer to mono-, di-, tri- and tetrasialogangliosides, respectively, and the numbers 1, 2, 3, etc. refer to the order of migration of the gangliosides on thin-layer chromatography. For example, the order of migration of monosialogangliosides is GM3>GM2>GM1. To indicate variations within the basic structures, further terms are added, e.g. GM1a, GD1b, etc. Glycosphingolipids having 0,1, 2, and 3 sialic acid residues linked to the inner galactose unit are termed asialo-(or 0-), a-, b- and c-series gangliosides, respectively, while gangliosides having sialic acid residues linked to the inner N-galactosamine residue are classified as α-series gangliosides. Pathways for the biosynthesis of the 0-, a-, b- and c-series of gangliosides involve sequential activities of sialyltransferases and glycosyltransferases as illustrated e.g. in Ledeen et al., 2015 (Ledeen, Robert W., and Gusheng Wu. “The multi-tasked life of GM1 ganglioside, a true factotum of nature.” Trends in biochemical sciences 40.7 (2015): 407-418). Further sialization of each of the series and in different positions in the carbohydrate chain can occur to give an increasingly complex and heterogeneous range of products, such as the α-series gangliosides with sialic acid residue(s) linked to the inner N-acetylgalactosamine residue.

In the context of the cell, gangliosides are transferred to the external leaflet of the plasma membrane by a transport system involving vesicle formation. Gangliosides are present and concentrated on cell surfaces, with the two hydrocarbon chains of the ceramide moiety embedded in the plasma membrane and the oligosaccharides located on the extracellular surface, where they present points of recognition for extracellular molecules or surfaces of neighbouring cells. The sialoglycan components of gangliosides extend out from the cell surface, where they can participate in intermolecular interactions. They function by recognizing specific molecules at the cell surface and by regulating the activities of proteins in the plasma membrane. Gangliosides also bind specifically to viruses and to bacterial toxins, such as those from botulinum, tetanus and cholera. For example, the specific cell surface receptor for the cholera toxin is ganglioside GM1 (or GM1a): Neu5Acα2-3 (Galβ1-3GalNAcβ1-4) Galβ1-4Glcβ1Cer.

BoNTs possess two independent binding regions in the H_{CC} domain for gangliosides and neuronal protein receptors. BoNT/A, /B, /E, /F and/G have a conserved ganglioside-binding site in the H_{CC} domain composed of a “E(Q) . . . H(K) . . . SXWY . . . G” motif, whereas BoNT/C, /D and/DC display two independent ganglioside-binding sites. (Lam, Kwok-Ho, et al. “Diverse binding modes, same goal: The receptor recognition mechanism of botulinum neurotoxin.” Progress in biophysics and molecular biology 117.2 (2015): 225-231.) Most BoNTs bind only to gangliosides that have an 2,3-linked N-acetylneuraminic acid residue (denoted Sia5) attached to Gal4 of the oligosaccharide core, whereas the corresponding ganglioside-binding pocket on TeNT can also bind to GM1a, a ganglioside lacking the Sia5 sugar residue. It has been shown that introducing a H1241K mutation into a recombinant BoNT/F confers GM1 binding ability (Benson, Marc A., et al. “Unique ganglioside recognition strategies for clostridial neurotoxins.” Journal of Biological Chemistry 286.39 (2011): 34015-34022). BoNT/D has been found to bind GM1a and GD1a (Kroken, Abby R., et al. “Novel ganglioside-mediated entry of botulinum neurotoxin serotype D into neurons.” Journal of Biological Chemistry 286.30 (2011): 26828-26837.)

A ganglioside may be GM1 (e.g. GM1a or GM1b), GM2, GM3 (e.g. NeuAc GM3 or NeuGc GM3), GM4, GD1a, GD1b, GalNAc-GD1a, GT1a, GT1b, GQ1b, GD2, or GD3.

Combining the data derived from ganglioside-deficient mice and biochemical assays, BoNT/A, E, F and G display a preference for the terminal NAcGal-Gal-NAcNeu moiety being present in GD1a and GT1b, whereas BoNT/B, C, D and TeNT require the disialyl motif found in GD1b, GT1b and GQ1b.

Thus, a ganglioside may comprise a terminal NAcGal-Gal-NAcNeu moiety or a disialyl motif. A ganglioside may comprise GD1a, GT1b, GD1b, GQ1b, or GM1 (Neu5Aca2-3 (Galβ1-3GalNAcβ1-4) Galβ1-4Glcβ1Cer). For example, a ganglioside may comprise GD1a, GT1b, GD1b, or GQ1b.

Suitable gangliosides may be selected from those described in WO 2018/060351.

Capture substrates comprising gangliosides may be employed at a concentration of 0.1-500 μM, for example 1-250 μM, 1-100 μM, 10-50 μM, or 20-30 μM, e.g. 24.1 μM.

A capture substrate preferably comprises a clostridial neurotoxin receptor polypeptide. The term “clostridial neurotoxin receptor polypeptide” may encompass a full-length clostridial neurotoxin receptor polypeptide or a portion thereof. The clostridial neurotoxin receptor polypeptide may be a neuronal clostridial neurotoxin receptor polypeptide, such as a full-length neuronal clostridial neurotoxin receptor polypeptide or a portion thereof. Preferably, the clostridial neurotoxin receptor polypeptide comprises (or consists of) an extracellular portion of a neuronal clostridial neurotoxin receptor, more preferably comprises (or consists of) an extracellular portion of a neuronal botulinum neurotoxin receptor.

Advantageously, by employing capture substrates comprising a clostridial neurotoxin receptor polypeptide (and optionally capture substrates comprising a ganglioside), the method of the invention may be improved. In particular, the method may allow for the improved determination as to whether a composition comprises an activity-altering property (e.g. unwanted polypeptide modification and/or degradation (e.g. oxidation)). For example, capture substrates comprising a clostridial neurotoxin receptor polypeptide (and optionally capture substrates comprising a ganglioside) may be more sensitive to an activity-altering property (e.g. unwanted polypeptide modification and/or degradation (e.g. oxidation)) of a clostridial neurotoxin when compared to an antibody capture substrate. Antibodies may be unable to detect or distinguish between clostridial neurotoxins that do or do not comprise an activity-altering property (e.g. unwanted polypeptide modification and/or degradation (e.g. oxidation)).

A capture substrate may comprise non-extracellular portions of the neuronal clostridial neurotoxin receptor but typically only those non-extracellular portions that are soluble in aqueous solution. In fact, it is preferred that any non-extracellular portions are absent, e.g. to improve solubility and thus ease of recombinant manufacturing and/or handling.

A neuronal clostridial neurotoxin receptor polypeptide may be a human neuronal clostridial neurotoxin receptor polypeptide (e.g. an extracellular portion thereof). A neuronal clostridial neurotoxin receptor polypeptide may be synaptic vesicle glycoprotein 2 (SV2) isoform A (SV2a), SV2 isoform B (SV2b), SV2 isoform C (SV2c), synaptotagmin | (SYT-I), or synaptotagmin II (SYT-II). Preferably, a neuronal clostridial neurotoxin receptor polypeptide comprises (or consists of) an extracellular portion of SV2a, SV2b, SV2c, SYT-I, or SYT-II. Thus, a capture substrate of the invention may comprise an extracellular portion of SV2a, SV2b, SV2c, SYT-I, or SYT-II. The polypeptide sequence of the neuronal clostridial neurotoxin receptor polypeptide (e.g. extracellular portion thereof) may be the same as the wild-type (e.g. human) polypeptide sequence. In another embodiment, the polypeptide of the neuronal clostridial neurotoxin receptor polypeptide (e.g. extracellular portion thereof) may comprise one or more modifications when compared to the wild-type (e.g. human) polypeptide sequence. Preferably, where the clostridial neurotoxin receptor polypeptide (e.g. extracellular portion thereof) is human SYT-II, the polypeptide sequence comprises a leucine 51 to phenylalanine substitution (L51F) as shown in SEQ ID NO: 21.

Where the clostridial neurotoxin composition comprises a clostridial neurotoxin comprising a BoNT/A H_{CC} (preferably H_{C}) domain, a capture substrate may comprise an extracellular portion of SV2c, SV2a, or SV2b, preferably SV2c. There are three SV2 isoforms found in humans, SV2a, SV2b, and SV2c however BoNT/A has the greatest affinity for SV2c. BoNT/A may bind specifically to the luminal domain 4 of SV2 (SV2-LD₄) via direct backbone-backbone interactions between a β-strand of SV2-LD₄ and a β-strand of BoNT H_{C}, and also through interactions with an N559-linked glycan.

Where the clostridial neurotoxin composition comprises a clostridial neurotoxin comprising a BoNT/B H_{CC} (preferably H_{C}) domain, a capture substrate may comprise an extracellular portion of SYT-II or SYT-I, preferably SYT-II. While BoNT/B binds to SYT-I and SYT-II, SYT-II is believed to be more abundant on human neuronal cells. Therefore, SYT-II may be a preferred neuronal clostridial neurotoxin receptor polypeptide for use in the present invention.

Where the clostridial neurotoxin composition comprises a clostridial neurotoxin comprising a BoNT/D H_{CC} (preferably H_{C}) domain, a capture substrate may comprise an extracellular portion of SV2c, SV2a, or SV2b.

Where the clostridial neurotoxin composition comprises a clostridial neurotoxin comprising a BoNT/E H_{CC} (preferably H_{C}) domain, a capture substrate may comprise an extracellular portion of SV2b or SV2c.

Where the clostridial neurotoxin composition comprises a clostridial neurotoxin comprising a BoNT/F H_{CC} (preferably H_{C}) domain, a capture substrate may comprise an extracellular portion of SV2c, SV2a, or SV2b.

Where the clostridial neurotoxin composition comprises a clostridial neurotoxin comprising a BoNT/G H_{CC} (preferably H_{C}) domain, a capture substrate may comprise an extracellular portion of SYT-II or SYT-I, preferably SYT-II.

An extracellular portion of SYT-II may comprise an extracellular portion of amino acid residues 1-75, 1-70, 1-65, 1-64, or 1-61 (preferably 1-61) of full-length SYT-II.

An extracellular portion of human SYT-II may refer to amino acid residues 1-61 of full-length human SYT-II. Positioning of residues may be determined by amino acid sequence alignment to SEQ ID NO: 23.

A full-length human SYT-II may comprise a polypeptide sequence having at least 70% sequence identity to SEQ ID NO: 23. In one embodiment, a full-length human SYT-II may comprise a polypeptide sequence having at least 80%, 85%, 90%, 95%, 99%, or 99.9% sequence identity to SEQ ID NO: 23. Preferably, a full-length human SYT-II may comprise SEQ ID NO: 23. A full-length human SYT-II may consist of a polypeptide sequence having at least 70% sequence identity to SEQ ID NO: 23. In one embodiment, a full-length human SYT-II may consist of a polypeptide sequence having at least 80%, 85%, 90%, 95%, 99%, or 99.9% sequence identity to SEQ ID NO: 23. Preferably, a full-length human SYT-II may consist of SEQ ID NO: 23.

An extracellular portion of mouse SYT-II may refer to amino acid residues 1-64 of full-length mouse SYT-II. Positioning of residues may be determined by amino acid sequence alignment to SEQ ID NO: 24.

A full-length mouse SYT-II may comprise a polypeptide sequence having at least 70% sequence identity to SEQ ID NO: 24. In one embodiment, a full-length mouse SYT-II may comprise a polypeptide sequence having at least 80%, 85%, 90%, 95%, 99%, or 99.9% sequence identity to SEQ ID NO: 24. Preferably, a full-length mouse SYT-II may comprise SEQ ID NO: 24. A full-length mouse SYT-II may consist of a polypeptide sequence having at least 70% sequence identity to SEQ ID NO: 24. In one embodiment, a full-length mouse SYT-II may consist of a polypeptide sequence having at least 80%, 85%, 90%, 95%, 99%, or 99.9% sequence identity to SEQ ID NO: 24. Preferably, a full-length mouse SYT-II may consist of SEQ ID NO: 24.

Thus, a capture substrate comprising an extracellular portion of mouse SYT-II may comprise a polypeptide sequence having at 70% sequence identity to SEQ ID NO: 22. In one embodiment, a capture substrate comprising an extracellular portion of mouse SYT-II may comprise a polypeptide sequence having at least 80%, 85%, 90%, 95%, 99%, or 99.9% sequence identity to SEQ ID NO: 22. Preferably, a capture substrate comprising an extracellular portion of mouse SYT-II may comprise SEQ ID NO: 22. Thus, a capture substrate comprising an extracellular portion of mouse SYT-II may consist of a polypeptide sequence having at 70% sequence identity to SEQ ID NO: 22. In one embodiment, a capture substrate comprising an extracellular portion of mouse SYT-II may consist of a polypeptide sequence having at least 80%, 85%, 90%, 95%, 99%, or 99.9% sequence identity to SEQ ID NO: 22. Preferably, a capture substrate comprising an extracellular portion of mouse SYT-II may consist of SEQ ID NO: 22.

The SYT-II is preferably human SYT-II.

An extracellular portion of human SYT-II may comprise a polypeptide sequence having at least 70% sequence identity to SEQ ID NO: 19. In one embodiment, an extracellular portion of human SYT-II may comprise a polypeptide sequence having at least 80%, 85%, 90%, 95%, 99%, or 99.9% sequence identity to SEQ ID NO: 19. Preferably, an extracellular portion of human SYT-II may comprise SEQ ID NO: 19. An extracellular portion of human SYT-II may consist of a polypeptide sequence having at least 70% sequence identity to SEQ ID NO: 19. In one embodiment, an extracellular portion of human SYT-II may consist of a polypeptide sequence having at least 80%, 85%, 90%, 95%, 99%, or 99.9% sequence identity to SEQ ID NO: 19. Preferably, an extracellular portion of human SYT-II may consist of SEQ ID NO: 19.

Thus, a capture substrate comprising an extracellular portion of human SYT-II may comprise a polypeptide sequence having at 70% sequence identity to SEQ ID NO: 18. In one embodiment, a capture substrate comprising an extracellular portion of human SYT-II may comprise a polypeptide sequence having at least 80%, 85%, 90%, 95%, 99%, or 99.9% sequence identity to SEQ ID NO: 18. Preferably, a capture substrate comprising an extracellular portion of human SYT-II may comprise SEQ ID NO: 18. Thus, a capture substrate comprising an extracellular portion of human SYT-II may consist of a polypeptide sequence having at 70% sequence identity to SEQ ID NO: 18. In one embodiment, a capture substrate comprising an extracellular portion of human SYT-II may consist of a polypeptide sequence having at least 80%, 85%, 90%, 95%, 99%, or 99.9% sequence identity to SEQ ID NO: 18. Preferably, a capture substrate comprising an extracellular portion of human SYT-II may consist of SEQ ID NO: 18.

As indicated, above, it is preferred that where the SYT-II is human SYT-II, the polypeptide sequence comprises a leucine 51 to phenylalanine substitution (L51F). In other words, the human SYT-II is preferably a modified human SYT-II. Positioning of residue 51 may be determined by amino acid sequence alignment to SEQ ID NO: 19. An extracellular portion of modified human SYT-II may comprise the L51F substitution and a polypeptide sequence having at least 70% sequence identity to SEQ ID NO: 21. In one embodiment, an extracellular portion of modified human SYT-II may comprise the L51F substitution and a polypeptide sequence having at least 80%, 85%, 90%, 95%, 99%, or 99.9% sequence identity to SEQ ID NO: 21. Preferably, an extracellular portion of modified human SYT-II may comprise SEQ ID NO: 21. An extracellular portion of modified human SYT-II may comprise the L51F substitution and consist of a polypeptide sequence having at least 70% sequence identity to SEQ ID NO: 21. In one embodiment, an extracellular portion of modified human SYT-II may comprise the L51F substitution and consist of a polypeptide sequence having at least 80%, 85%, 90%, 95%, 99%, or 99.9% sequence identity to SEQ ID NO: 21. Preferably, an extracellular portion of modified human SYT-II may consist of SEQ ID NO: 21. An extracellular portion of modified human SYT-II may comprise the L51F substitution and be encoded by a nucleotide sequence comprising at least 70% sequence identity to SEQ ID NO: 50. In one embodiment, an extracellular portion of modified human SYT-II may comprise the L51F substitution and be encoded by a nucleotide sequence comprising at least 80%, 85%, 90%, 95%, 99%, or 99.9% sequence identity to SEQ ID NO: 50. Preferably, an extracellular portion of modified human SYT-II may comprise the L51F substitution and be encoded by a nucleotide sequence comprising (more preferably consisting of) SEQ ID NO: 50.

Thus, a capture substrate comprising an extracellular portion of modified human SYT-II may comprise the L51F substitution and a polypeptide sequence having at least 70% sequence identity to SEQ ID NO: 20. In one embodiment, a capture substrate comprising an extracellular portion of modified human SYT-II may comprise the L51F substitution and a polypeptide sequence having at least 80%, 85%, 90%, 95%, 99%, or 99.9% sequence identity to SEQ ID NO: 20. Preferably, a capture substrate comprising an extracellular portion of modified human SYT-II may comprise SEQ ID NO: 20. Thus, a capture substrate comprising an extracellular portion of modified human SYT-II may comprise the L51F substitution and consist of a polypeptide sequence having at least 70% sequence identity to SEQ ID NO: 20. In one embodiment, a capture substrate comprising an extracellular portion of modified human SYT-II may comprise the L51F substitution and consist of a polypeptide sequence having at least 80%, 85%, 90%, 95%, 99%, or 99.9% sequence identity to SEQ ID NO: 20. Preferably, a capture substrate comprising an extracellular portion of modified human SYT-II may consist of SEQ ID NO: 20.

An extracellular portion of SYT-I may comprise an extracellular portion of amino acid residues 1-57 (e.g. amino acid residues 33-54) of full-length SYT-I.

An extracellular portion of mouse SYT-I may comprise an extracellular portion of amino acid residues 1-59 of full-length mouse SYT-I. An extracellular portion of mouse SYT-I may refer to amino acid residues 1-59 of full-length mouse SYT-I. Positioning of residues may be determined by amino acid sequence alignment to SEQ ID NO: 82.

An extracellular portion of mouse SYT-I may comprise a polypeptide sequence having at least 70% sequence identity to amino acid residues 1-59 of SEQ ID NO: 82. In one embodiment, an extracellular portion of mouse SYT-I may comprise a polypeptide sequence having at least 80%, 85%, 90%, 95%, 99%, or 99.9% sequence identity to amino acid residues 1-59 of SEQ ID NO: 82. Preferably, an extracellular portion of mouse SYT-I may comprise amino acid residues 1-59 of SEQ ID NO: 82. An extracellular portion of mouse SYT-I may consist of a polypeptide sequence having at least 70% sequence identity to amino acid residues 1-59 of SEQ ID NO: 82. In one embodiment, an extracellular portion of mouse SYT-I may consist of a polypeptide sequence having at least 80%, 85%, 90%, 95%, 99%, or 99.9% sequence identity to amino acid residues 1-59 of SEQ ID NO: 82. Preferably, an extracellular portion of mouse SYT-I may consist of amino acid residues 1-59 of SEQ ID NO: 82.

A full-length mouse SYT-I may comprise a polypeptide sequence having at least 70% sequence identity to SEQ ID NO: 82. In one embodiment, a full-length mouse SYT-I may comprise a polypeptide sequence having at least 80%, 85%, 90%, 95%, 99%, or 99.9% sequence identity to SEQ ID NO: 82. Preferably, a full-length mouse SYT-I may comprise SEQ ID NO: 82. A full-length mouse SYT-I may consist of a polypeptide sequence having at least 70% sequence identity to SEQ ID NO: 82. In one embodiment, a full-length mouse SYT-I may consist of a polypeptide sequence having at least 80%, 85%, 90%, 95%, 99%, or 99.9% sequence identity to SEQ ID NO: 82. Preferably, a full-length mouse SYT-I may consist of SEQ ID NO: 82.

An extracellular portion of mouse SYT-I may comprise a polypeptide sequence having at least 70% sequence identity to SEQ ID NO: 83. In one embodiment, an extracellular portion of mouse SYT-I may comprise a polypeptide sequence having at least 80%, 85%, 90%, 95%, 99%, or 99.9% sequence identity to SEQ ID NO: 83. Preferably, an extracellular portion of mouse SYT-I may comprise SEQ ID NO: 83. An extracellular portion of mouse SYT-I may consist of a polypeptide sequence having at least 70% sequence identity to SEQ ID NO: 83. In one embodiment, an extracellular portion of mouse SYT-I may consist of a polypeptide sequence having at least 80%, 85%, 90%, 95%, 99%, or 99.9% sequence identity to SEQ ID NO: 83. Preferably, an extracellular portion of mouse SYT-I may consist of SEQ ID NO: 83.

Thus, a capture substrate comprising an extracellular portion of mouse SYT-I may comprise a polypeptide sequence having at least 70% sequence identity to SEQ ID NO: 84. In one embodiment, a capture substrate comprising an extracellular portion of mouse SYT-I may comprise a polypeptide sequence having at least 80%, 85%, 90%, 95%, 99%, or 99.9% sequence identity to SEQ ID NO: 84. Preferably, a capture substrate comprising an extracellular portion of mouse SYT-I may comprise SEQ ID NO: 84. Thus, a capture substrate comprising an extracellular portion of mouse SYT-I may consist of a polypeptide sequence having at least 70% sequence identity to SEQ ID NO: 84. In one embodiment, a capture substrate comprising an extracellular portion of mouse SYT-I may consist of a polypeptide sequence having at least 80%, 85%, 90%, 95%, 99%, or 99.9% sequence identity to SEQ ID NO: 84. Preferably, a capture substrate comprising an extracellular portion of mouse SYT-I may consist of SEQ ID NO: 84.

The SYT-I is preferably human SYT-I. An extracellular portion of human SYT-I may refer to amino acid residues 1-57 (e.g. amino acid residues 33-54) of full-length human SYT-I. Thus, an extracellular potin of human SYT-I may comprise amino acid residues 1-57 (e.g. amino acid residues 33-54) of full-length human SYT-I. Positioning of residues may be determined by amino acid sequence alignment to SEQ ID NO: 28.

An extracellular portion of human SYT-I may comprise a polypeptide sequence having at least 70% sequence identity to amino acid residues 1-57 (e.g. amino acid residues 33-54) of SEQ ID NO: 28. In one embodiment, an extracellular portion of human SYT-I may comprise a polypeptide sequence having at least 80%, 85%, 90%, 95%, 99%, or 99.9% sequence identity to amino acid residues 1-57 (e.g. amino acid residues 33-54) of SEQ ID NO: 28. Preferably, an extracellular portion of human SYT-I may comprise amino acid residues 1-57 (e.g. amino acid residues 33-54) of SEQ ID NO: 28. An extracellular portion of human SYT-I may consist of a polypeptide sequence having at least 70% sequence identity to amino acid residues 1-57 (e.g. amino acid residues 33-54) of SEQ ID NO: 28. In one embodiment, an extracellular portion of human SYT-I may consist of a polypeptide sequence having at least 80%, 85%, 90%, 95%, 99%, or 99.9% sequence identity to amino acid residues 1-57 (e.g. amino acid residues 33-54) of SEQ ID NO: 28. Preferably, an extracellular portion of human SYT-I may consist of amino acid residues 1-57 (e.g. amino acid residues 33-54) of SEQ ID NO: 28.

Preferably an extracellular portion of SYT-I may comprise an extracellular portion of amino acid residues 1-60 (e.g. amino acid residues 33-54) of full-length SYT-I. An extracellular portion of human SYT-I may refer to amino acid residues 1-60 (e.g. amino acid residues 33-54) of full-length human SYT-I. Positioning of residues may be determined by amino acid sequence alignment to SEQ ID NO: 28.

An extracellular portion of human SYT-I may comprise a polypeptide sequence having at least 70% sequence identity to amino acid residues 1-60 (e.g. amino acid residues 33-54) of SEQ ID NO: 28. In one embodiment, an extracellular portion of human SYT-I may comprise a polypeptide sequence having at least 80%, 85%, 90%, 95%, 99%, or 99.9% sequence identity to amino acid residues 1-60 (e.g. amino acid residues 33-54) of SEQ ID NO: 28. Preferably, an extracellular portion of human SYT-I may comprise amino acid residues 1-60 (e.g. amino acid residues 33-54) of SEQ ID NO: 28. An extracellular portion of human SYT-I may consist of a polypeptide sequence having at least 70% sequence identity to amino acid residues 1-60 (e.g. amino acid residues 33-54) of SEQ ID NO: 28. In one embodiment, an extracellular portion of human SYT-I may consist of a polypeptide sequence having at least 80%, 85%, 90%, 95%, 99%, or 99.9% sequence identity to amino acid residues 1-60 (e.g. amino acid residues 33-54) of SEQ ID NO: 28. Preferably, an extracellular portion of human SYT-I may consist of amino acid residues 1-60 (e.g. amino acid residues 33-54) of SEQ ID NO: 28.

A full-length human SYT-I may comprise a polypeptide sequence having at least 70% sequence identity to SEQ ID NO: 28. In one embodiment, a full-length human SYT-I may comprise a polypeptide sequence having at least 80%, 85%, 90%, 95%, 99%, or 99.9% sequence identity to SEQ ID NO: 28. Preferably, a full-length human SYT-I may comprise SEQ ID NO: 28. A full-length human SYT-I may consist of a polypeptide sequence having at least 70% sequence identity to SEQ ID NO: 28. In one embodiment, a full-length human SYT-I may consist of a polypeptide sequence having at least 80%, 85%, 90%, 95%, 99%, or 99.9% sequence identity to SEQ ID NO: 28. Preferably, a full-length human SYT-I may consist of SEQ ID NO: 28.

An extracellular portion of human SYT-I may comprise a polypeptide sequence having at least 70% sequence identity to SEQ ID NO: 80. In one embodiment, an extracellular portion of human SYT-I may comprise a polypeptide sequence having at least 80%, 85%, 90%, 95%, 99%, or 99.9% sequence identity to SEQ ID NO: 80. Preferably, an extracellular portion of human SYT-I may comprise SEQ ID NO: 80. An extracellular portion of human SYT-I may consist of a polypeptide sequence having at least 70% sequence identity to SEQ ID NO: 80. In one embodiment, an extracellular portion of human SYT-I may consist of a polypeptide sequence having at least 80%, 85%, 90%, 95%, 99%, or 99.9% sequence identity to SEQ ID NO: 80. Preferably, an extracellular portion of human SYT-I may consist of SEQ ID NO: 80.

Thus, a capture substrate comprising an extracellular portion of human SYT-I may comprise a polypeptide sequence having at least 70% sequence identity to SEQ ID NO: 81. In one embodiment, a capture substrate comprising an extracellular portion of human SYT-I may comprise a polypeptide sequence having at least 80%, 85%, 90%, 95%, 99%, or 99.9% sequence identity to SEQ ID NO: 81. Preferably, a capture substrate comprising an extracellular portion of human SYT-I may comprise SEQ ID NO: 81. Thus, a capture substrate comprising an extracellular portion of human SYT-I may consist of a polypeptide sequence having at least 70% sequence identity to SEQ ID NO: 81. In one embodiment, a capture substrate comprising an extracellular portion of human SYT-I may consist of a polypeptide sequence having at least 80%, 85%, 90%, 95%, 99%, or 99.9% sequence identity to SEQ ID NO: 81. Preferably, a capture substrate comprising an extracellular portion of human SYT-I may consist of SEQ ID NO: 81.

An extracellular portion of SV2a may comprise an extracellular portion of amino acid residues 469-599 (e.g. amino acid residues 469-598 or 469-595) of full-length SV2a. An extracellular portion of human SV2a may refer to amino acid residues 469-599 (e.g. amino acid residues 469-598 or 469-595) of full-length human SV2a. Positioning of residues may be determined by amino acid sequence alignment to SEQ ID NO: 25.

A full-length human SV2a may comprise a polypeptide sequence having at least 70% sequence identity to SEQ ID NO: 25. In one embodiment, a full-length human SV2a may comprise a polypeptide sequence having at least 80%, 85%, 90%, 95%, 99%, or 99.9% sequence identity to SEQ ID NO: 25. Preferably, a full-length human SV2a may comprise SEQ ID NO: 25. A full-length human SV2a may consist of a polypeptide sequence having at least 70% sequence identity to SEQ ID NO: 25. In one embodiment, a full-length human SV2a may consist of a polypeptide sequence having at least 80%, 85%, 90%, 95%, 99%, or 99.9% sequence identity to SEQ ID NO: 25. Preferably, a full-length human SV2a may consist of SEQ ID NO: 25.

An extracellular portion of SV2b may comprise an extracellular portion of amino acid residues 410-539 of full-length SV2b. An extracellular portion of human SV2b may refer to amino acid residues 410-539 of full-length human SV2b. Positioning of residues may be determined by amino acid sequence alignment to SEQ ID NO: 26.

A full-length human SV2b may comprise a polypeptide sequence having at least 70% sequence identity to SEQ ID NO: 26. In one embodiment, a full-length human SV2b may comprise a polypeptide sequence having at least 80%, 85%, 90%, 95%, 99%, or 99.9% sequence identity to SEQ ID NO: 26. Preferably, a full-length human SV2b may comprise SEQ ID NO: 26. A full-length human SV2b may consist of a polypeptide sequence having at least 70% sequence identity to SEQ ID NO: 26. In one embodiment, a full-length human SV2b may consist of a polypeptide sequence having at least 80%, 85%, 90%, 95%, 99%, or 99.9% sequence identity to SEQ ID NO: 26. Preferably, a full-length human SV2b may consist of SEQ ID NO: 26.

An extracellular portion of SV2c may comprise an extracellular portion of amino acid residues 400-600, 450-590, 460-580, 470-570, or 500-570 of full-length SV2c. An extracellular portion of human SV2c may comprise at least luminal domain 4 (e.g. amino acid residues 519-563 of SV2c). Thus, an extracellular portion of human SV2c may refer to amino acid residues 519-563 of full-length human SV2c. The extracellular portion of human SV2c comprising at least luminal domain 4 may comprise 473-567 of full-length human SV2c. Positioning of residues may be determined by amino acid sequence alignment to SEQ ID NO: 27.

A full-length human SV2c may comprise a polypeptide sequence having at least 70% sequence identity to SEQ ID NO: 27. In one embodiment, a full-length human SV2c may comprise a polypeptide sequence having at least 80%, 85%, 90%, 95%, 99%, or 99.9% sequence identity to SEQ ID NO: 27. Preferably, a full-length human SV2c may comprise SEQ ID NO: 27. A full-length human SV2c may consist of a polypeptide sequence having at least 70% sequence identity to SEQ ID NO: 27. In one embodiment, a full-length human SV2c may consist of a polypeptide sequence having at least 80%, 85%, 90%, 95%, 99%, or 99.9% sequence identity to SEQ ID NO: 27. Preferably, a full-length human SV2c may consist of SEQ ID NO: 27.

An extracellular portion of human SV2c may comprise a polypeptide sequence having at least 70% sequence identity to SEQ ID NO: 30. In one embodiment, an extracellular portion of human SV2c may comprise a polypeptide sequence having at least 80%, 85%, 90%, 95%, 99%, or 99.9% sequence identity to SEQ ID NO: 30. Preferably, an extracellular portion of human SV2c may comprise SEQ ID NO: 30. An extracellular portion of human SV2c may consist of a polypeptide sequence having at least 70% sequence identity to SEQ ID NO: 30. In one embodiment, an extracellular portion of human SV2c may consist of a polypeptide sequence having at least 80%, 85%, 90%, 95%, 99%, or 99.9% sequence identity to SEQ ID NO: 30. Preferably, an extracellular portion of human SV2c may consist of SEQ ID NO: 30.

Thus, a capture substrate comprising an extracellular portion of human SV2c may comprise a polypeptide sequence having at least 70% sequence identity to SEQ ID NO: 29. In one embodiment, a capture substrate comprising an extracellular portion of human SV2c may comprise a polypeptide sequence having at least 80%, 85%, 90%, 95%, 99%, or 99.9% sequence identity to SEQ ID NO: 29. Preferably, a capture substrate comprising an extracellular portion of human SV2c may comprise SEQ ID NO: 29. Thus, a capture substrate comprising an extracellular portion of human SV2c may consist of a polypeptide sequence having at least 70% sequence identity to SEQ ID NO: 29. In one embodiment, a capture substrate comprising an extracellular portion of human SV2c may consist of a polypeptide sequence having at least 80%, 85%, 90%, 95%, 99%, or 99.9% sequence identity to SEQ ID NO: 29. Preferably, a capture substrate comprising an extracellular portion of human SV2c may consist of SEQ ID NO: 29.

A capture substrate comprising an extracellular portion of human SV2c may be encoded by a nucleotide sequence comprising at least 70% sequence identity to SEQ ID NO: 51. In one embodiment, a capture substrate comprising an extracellular portion of human SV2c may be encoded by a nucleotide sequence comprising at least 80%, 85%, 90%, 95%, 99%, or 99.9% sequence identity to SEQ ID NO: 51. Preferably, a capture substrate comprising an extracellular portion of human SV2c may be encoded by a nucleotide sequence comprising (more preferably consisting of) SEQ ID NO: 51.

Thus, a capture substrate comprising an extracellular portion of human SV2c may comprise a polypeptide sequence having at least 70% sequence identity to SEQ ID NO: 40. In one embodiment, a capture substrate comprising an extracellular portion of human SV2c may comprise a polypeptide sequence having at least 80%, 85%, 90%, 95%, 99%, or 99.9% sequence identity to SEQ ID NO: 40. Preferably, a capture substrate comprising an extracellular portion of human SV2c may comprise SEQ ID NO: 40. Thus, a capture substrate comprising an extracellular portion of human SV2c may consist of a polypeptide sequence having at least 70% sequence identity to SEQ ID NO: 40. In one embodiment, a capture substrate comprising an extracellular portion of human SV2c may consist of a polypeptide sequence having at least 80%, 85%, 90%, 95%, 99%, or 99.9% sequence identity to SEQ ID NO: 40. Preferably, a capture substrate comprising an extracellular portion of human SV2c may consist of SEQ ID NO: 40.

Of the capture substrates comprising an extracellular portion of human SV2c, SEQ ID NO: 40 is most preferred.

Prior to carrying out a method of the invention, capture substrates may be immobilised on a solid support. Such immobilisation may be achieved using any means known in the art, preferably by way of a tag present on the capture substrate (e.g. a GST tag) and an appropriate binding partner (e.g. glutathione or a derivative thereof) present on the solid support. Other suitable tags are known in the at, such as His-tags. Suitable solid supports are known in the art. Advantageously, this allows for convenient washing of any capture substrate-clostridial neurotoxin complex, and easy removal of supernatant while minimising loss of such complexes. A solid support may be a plastic support, such as an inner surface (in normal use) of a plate (e.g. a multi-well plate, such as a microtitre plate), a column, or a tube (e.g. microcentrifuge tube). Preferably, a solid support is an inner surface (in normal use) of a multi-well plate. Multi-well plates are convenient for handling a large number of samples and lend themselves to use in high-throughput techniques.

A capture substrate of the invention may comprise a post-translational modification, such as a glycosylation. The glycosylation is preferably N-linked glycosylation. The N-linked glycosylation preferably occurs at an asparagine residue present in a clostridial neurotoxin receptor polypeptide comprised in the capture substrate. Preferably, the clostridial neurotoxin receptor polypeptide comprises an extracellular portion of SV2c glycosylated at N559. Positioning of residues may be determined by amino acid sequence alignment to SEQ ID NO: 27. A glycosylation may comprise a Man-5 glycan, a G0f glycan, a G1f glycan, or a G2f glycan. The glycosylation preferably further comprises N-acetyl glucosamine (GlcNAc), e.g. G0f-GlcNAc.

A capture substrate comprising a post-translational modification may be produced by expressing a nucleic acid encoding said capture substrate in a suitable host cell. The capture substrate may then be isolated from said host cell using standard techniques. For example, the host cell may be a mammalian cell (e.g. from a mammalian cell line), preferably a human host cell, such as a human cell line host cell. Such host cells may be particularly advantageous when assaying clostridial neurotoxin compositions for human therapeutic and/or cosmetic use, as the capture substrates preferably exhibit post-translational modifications that are more similar to those found in a human when compared to capture substrates produced using alternative methods, such as those produced via prokaryotic (e.g. E. coli) expression.

Advantageously, when the capture substrates comprise an SV2c (e.g. an extracellular portion thereof) comprising a glycosylation, said capture substrate may allow for improved (e.g. more sensitive) differentiation between oxidised and non-oxidised clostridial neurotoxin polypeptides (when compared to an equivalent capture substrate that does not comprise the glycosylation).

An appropriate host cell for producing a capture substrate comprising a post-translational modification may be a HEK293 cell, a Chinese hamster ovary (CHO) cell, or a Drosophila melanogaster cell, preferably a HEK293 cell. In some embodiments, a nucleic acid encoding a capture substrate may be expressed using a Drosophila expression system known in the art.

A nucleic acid may comprise at least 70% sequence identity to SEQ ID NO: 50. In one embodiment, a nucleic acid may comprise at least 80%, 85%, 90%, 95%, 99%, or 99.9% sequence identity to SEQ ID NO: 50. Preferably, a nucleic acid may comprise (more preferably consist of) SEQ ID NO: 50.

A nucleic acid may comprise at least 70% sequence identity to SEQ ID NO: 51. In one embodiment, a nucleic acid may comprise at least 80%, 85%, 90%, 95%, 99%, or 99.9% sequence identity to SEQ ID NO: 51. Preferably, a nucleic acid may comprise (more preferably consist of) SEQ ID NO: 51.

A cleavable substrate for use in the present invention is any substrate capable of being cleaved by a clostridial neurotoxin. Suitably, the cleavable substrate is selected for the clostridial neurotoxin and/or clostridial neurotoxin L-chain being assayed. A cleavable substrate comprises a clostridial neurotoxin cleavage site, e.g. a scissile bond of a SNARE. Suitable cleavable substrates and components thereof may be those described in WO 2018/075783 A2, which is incorporated herein by reference.

The clostridial neurotoxin cleavage site may further comprise a number of amino acids N-terminal and/or C-terminal to the scissile bond in a SNARE, preferably N-terminal and C-terminal. Thus, a clostridial neurotoxin cleavage site may comprise at least amino acid residues P3P2P1P1′P2′P3′ of a SNARE, wherein P1 and P1′ are the residues located N-terminal and C-terminal to the scissile bond (scissile peptide bond) cleaved by the clostridial neurotoxin, such as Gln197 and Arg198 of SNAP-25 cleaved by an L-chain of BoNT/A.

Thus, the clostridial neurotoxin cleavage site may further comprise at least 2 (e.g. the cleavage site comprises at least P3P2P1P1′), 5, 10, 15, 20, 30, 40, 50, or 100 amino acid residues N-terminal of the amino acid residues forming a scissile bond in a SNARE. The clostridial neurotoxin cleavage site may further comprise at least 2 (e.g. the cleavage site comprises at least P1P1′P2′P3′), 5, 10, 15, 20, 25, 30, 40, 50, or 100 amino acid residues C-terminal of the of the amino acid residues forming a scissile bond in a SNARE. For example, the clostridial neurotoxin cleavage site may further comprise: up to 5 amino acid residues N-terminal and up to 5 amino acid residues C-terminal of the amino acid residues forming a scissile bond in the SNARE; up to 10 amino acid residues N-terminal and up to 10 amino acid residues C-terminal of the amino acid residues forming a scissile bond in the SNARE; up to 25 amino acid residues N-terminal and up to 25 amino acid residues C-terminal of the amino acid residues forming a scissile bond in the SNARE; up to 50 amino acid residues N-terminal and up to 50 amino acid residues C-terminal of the amino acid residues forming a scissile bond in the SNARE; or up to 100 amino acid residues N-terminal and up to 100 amino acid residues C-terminal of the amino acid residues forming a scissile bond in the SNARE. For the avoidance of doubt, the term “up to” as used in this context encompasses the number indicated, e.g. “up to 5 amino acid residues” encompasses “5 amino acid residues”.

Preferably, a clostridial neurotoxin cleavage site comprises 8 amino acid residues C-terminal of the amino acid residues forming a scissile bond in the SNARE and up to 55 amino acid residues N-terminal of the amino acid residues forming a scissile bond in the SNARE (e.g. a clostridial neurotoxin cleavage site may comprise up to a 65 amino acid portion of a SNARE).

A clostridial neurotoxin cleavage site comprising a scissile bond of a SNARE may comprise (or consist of) at least 6, 7, 8, 9, 10, 20, 30, 40, 50, 60, 70, 80, 90, or 100 amino acid residues of a SNARE as described herein, preferably at least 55 amino acid residues of a SNARE as described herein. A clostridial neurotoxin cleavage site comprising a scissile bond of a SNARE may comprise (or consist of)≤200, ≤150, ≤100, ≤90, ≤80, ≤70, ≤60, ≤50, ≤40, ≤30, ≤20, ≤10, ≤9, ≤8, or ≤7 amino acid residues of a SNARE as described herein. For example, a clostridial neurotoxin cleavage site comprising a scissile bond of a SNARE may comprise (or consist of) 6-200, 10-150, 20-100, 50-85, 55-80 or 60-75 amino acid residues of a SNARE as described herein. Preferably, a clostridial neurotoxin cleavage site comprising a scissile bond of a SNARE may comprise (or consist of) 60-70 amino acid residues of a SNARE as described herein.

Thus, in some embodiments, the clostridial neurotoxin cleavage site is from a SNARE (e.g. is a fragment of a SNARE). A SNARE may be a SNAP-25, a synaptobrevin (VAMP), or a Syntaxin. Thus, a clostridial neurotoxin cleavage site may comprise a scissile bond of a SNAP-25, a synaptobrevin (VAMP), or a Syntaxin.

A SNARE for use in the invention is preferably a SNAP-25. Examples of SNAP-25 polypeptides include NCBI Gene ID: 6616, NCBI Reference Sequence: NP 570824.1 (human SNAP25b) and NCBI Reference Sequence: NP_112253.1 (rat SNAP25b). It is preferred that the SNAP-25 is human SNAP-25. SNAP-25 may comprise a polypeptide sequence having at least 70% sequence identity to SEQ ID NO: 4. In one embodiment, a SNAP-25 may comprise a polypeptide sequence having at least 80%, 85%, 90%, 95%, 99%, or 99.9% sequence identity to SEQ ID NO: 4. Preferably, a SNAP-25 may comprise SEQ ID NO: 4. SNAP-25 may consist of a polypeptide sequence having at least 70% sequence identity to SEQ ID NO: 4. In one embodiment, a SNAP-25 may consist of a polypeptide sequence having at least 80%, 85%, 90%, 95%, 99%, or 99.9% sequence identity to SEQ ID NO: 4. Preferably, a SNAP-25 may consist of SEQ ID NO: 4.

A scissile bond of SNAP-25 (SEQ ID NO: 4) cleaved by an L-chain of BoNT/A may be Gln197-Arg198. A scissile bond of SNAP-25 (SEQ ID NO: 4) cleaved by an L-chain of BoNT/C1 may be Arg198-Ala199. A scissile bond of SNAP-25 (SEQ ID NO: 4) cleaved by an L-chain of BoNT/E may be Arg180-Ile181.

A clostridial neurotoxin cleavage site comprising a SNAP-25 scissile bond may comprise a polypeptide sequence having at least 70% sequence identity to SEQ ID NO: 4. In one embodiment, a clostridial neurotoxin cleavage site comprising a SNAP-25 scissile bond may comprise a polypeptide sequence having at least 80%, 85%, 90%, 95%, 99%, or 99.9% sequence identity to SEQ ID NO: 4. Preferably, a clostridial neurotoxin cleavage site comprising a SNAP-25 scissile bond may comprise SEQ ID NO: 4. A clostridial neurotoxin cleavage site comprising a SNAP-25 scissile bond may consist of a polypeptide sequence having at least 70% sequence identity to SEQ ID NO: 4. In one embodiment, a clostridial neurotoxin cleavage site comprising a SNAP-25 scissile bond may consist of a polypeptide sequence having at least 80%, 85%, 90%, 95%, 99%, or 99.9% sequence identity to SEQ ID NO: 4. Preferably, a clostridial neurotoxin cleavage site comprising a SNAP-25 scissile bond may consist of SEQ ID NO: 4. Preferably, the N-terminal methionine residue (residue 1) of SEQ ID NO: 4 is not present when the clostridial neurotoxin cleavage site is comprised in a cleavable substrate of the invention.

A clostridial neurotoxin cleavage site comprising a SNAP-25 scissile bond may comprise a polypeptide sequence having at least 70% sequence identity to SEQ ID NO: 5. In one embodiment, a clostridial neurotoxin cleavage site comprising a SNAP-25 scissile bond may comprise a polypeptide sequence having at least 80%, 85%, 90%, 95%, 99%, or 99.9% sequence identity to SEQ ID NO: 5. Preferably, a clostridial neurotoxin cleavage site comprising a SNAP-25 scissile bond may comprise SEQ ID NO: 5. A clostridial neurotoxin cleavage site comprising a SNAP-25 scissile bond may consist of a polypeptide sequence having at least 70% sequence identity to SEQ ID NO: 5. In one embodiment, a clostridial neurotoxin cleavage site comprising a SNAP-25 scissile bond may consist of a polypeptide sequence having at least 80%, 85%, 90%, 95%, 99%, or 99.9% sequence identity to SEQ ID NO: 5. Preferably, a clostridial neurotoxin cleavage site comprising a SNAP-25 scissile bond may consist of SEQ ID NO: 5.

A SNARE may be a VAMP. A VAMP may be a VAMP1, VAMP2, VAMP3, VAMP4, VAMP5, or YKT6. The VAMP may be a human VAMP. Exemplary VAMP polypeptide sequences are shown in the table below. Thus, a VAMP may comprise a polypeptide sequence having at least 70% sequence identity to any of SEQ ID NOs: 31-36. In one embodiment, a VAMP may comprise a polypeptide sequence having at least 80%, 85%, 90%, 95%, 99%, or 99.9% sequence identity to any of SEQ ID NOs: 31-36. Preferably, a VAMP may comprise any of SEQ ID NOs: 31-36. Thus, a VAMP may consist of a polypeptide sequence having at least 70% sequence identity to any of SEQ ID NOs: 31-36. In one embodiment, a VAMP may consist of a polypeptide sequence having at least 80%, 85%, 90%, 95%, 99%, or 99.9% sequence identity to any of SEQ ID NOs: 31-36. Preferably, a VAMP may consist of any of SEQ ID NOs: 31-36.

VAMP scissile bonds cleaved by L-chains of the clostridial neurotoxins indicated are shown in the table below:

| | SEQ | | | BoNT/ | | |
|---|---|---|---|---|---|---|
| | ID | BoNT/ | BoNT/ | B & | BoNT/ | BoNT/ |
| VAMP | NO | F | D | TeNT | G | X |
| VAMP1_human | 31 | Gln60- | Lys61- | Gln78- | Ala83- | Arg68- |
| (P23763) | | Lys61 | Leu62 | Phe79 | Ala84 | Ala69 |
| VAMP2_human | 32 | Gln58- | Lys59- | Gln76- | Ala81- | Arg66- |
| (P63027) | | Lys59 | Leu60 | Phe77 | Ala82 | Ala67 |
| VAMP3_human | 33 | Gln41- | Lys42- | Gln59- | Ala64- | Arg49- |
| (Q15836) | | Lys42 | Leu43 | Phe60 | Ala65 | Ala50 |
| VAMP4_human | 34 | Not | Not | Not | Not | Lys87- |
| (O75379) | | cleaved | cleaved | cleaved | cleaved | Ser88 |
| VAMP5_human | 35 | Not | Not | Not | Not | Arg40- |
| (O95183) | | cleaved | cleaved | cleaved | cleaved | Ser41 |
| YKT6_human | 36 | Not | Not | Not | Not | Lys173- |
| (O15498) | | cleaved | cleaved | cleaved | cleaved | Ser174 |

Human VAMP1, VAMP2, and VAMP3 may be cleaved by an L-chain of BoNT/B, BoNT/D, BoNT/F, BoNT/G, BoNT/X, and TeNT. VAMP4, VAMP5, and YKT6 may be cleaved by an L-chain of BoNT/X.

A SNARE may be a Syntaxin. A Syntaxin may be Syntaxin 1A or Syntaxin 1B.

Syntaxin 1A may comprise a polypeptide sequence having at least 70% sequence identity to SEQ ID NO: 37. In one embodiment, Syntaxin 1A may comprise a polypeptide sequence having at least 80%, 85%, 90%, 95%, 99%, or 99.9% sequence identity to SEQ ID NO: 37. Preferably, Syntaxin 1A may comprise SEQ ID NO: 37. Syntaxin 1A may consist of a polypeptide sequence having at least 70% sequence identity to SEQ ID NO: 37. In one embodiment, Syntaxin 1A may consist of a polypeptide sequence having at least 80%, 85%, 90%, 95%, 99%, or 99.9% sequence identity to SEQ ID NO: 37. Preferably, Syntaxin 1A may consist of SEQ ID NO: 37.

Syntaxin 1B may comprise a polypeptide sequence having at least 70% sequence identity to SEQ ID NO: 38. In one embodiment, Syntaxin 1B may comprise a polypeptide sequence having at least 80%, 85%, 90%, 95%, 99%, or 99.9% sequence identity to SEQ ID NO: 38. Preferably, Syntaxin 1B may comprise SEQ ID NO: 38. Syntaxin 1B may consist of a polypeptide sequence having at least 70% sequence identity to SEQ ID NO: 38. In one embodiment, Syntaxin 1B may consist of a polypeptide sequence having at least 80%, 85%, 90%, 95%, 99%, or 99.9% sequence identity to SEQ ID NO: 38. Preferably, Syntaxin 1B may consist of SEQ ID NO: 38.

Syntaxin 1A and Syntaxin 1B may be cleaved by BoNT/C1. A scissile bond of Syntaxin 1A (SEQ ID NO: 37) cleaved by an L-chain of BoNT/C1 may be Lys253-Ala254. A scissile bond of Syntaxin 1B (SEQ ID NO: 38) cleaved by an L-chain of BoNT/C1 may be Lys252-Ala253.

In embodiments where a polypeptide sequence of a clostridial neurotoxin cleavage site varies from a recited SNARE polypeptide sequence (SEQ ID NO) by way of sequence identity, said clostridial neurotoxin cleavage site still comprises the relevant scissile bond of said SNARE polypeptide sequence.

A cleavable substrate may further comprise an element for indicating the presence or absence of cleavage of the cleavable substrate at the clostridial neurotoxin cleavage site. The element may comprise two different states, one state indicating the presence of cleavage and another state indicating the absence of cleavage. The element may comprise a single module or multiple modules (preferably two modules). The multiple modules may be different from one another. Preferably, the element comprises two modules, wherein a first module is N-terminal to a linker comprising (or consisting of) a clostridial neurotoxin cleavage site and a second module is C-terminal to a linker comprising (or consisting of) a clostridial neurotoxin cleavage site. The cleavable substrate is preferably a single-chain polypeptide (e.g. fusion polypeptide) when in an uncleaved form.

Advantageously, cleavable substrates comprising an element for indicating the presence or absence of cleavage may exhibit improved properties, e.g. when used in a method of the invention. In contrast, methods that employ cleavable substrates and an antibody that bind to the cleaved form (and not the uncleaved form) of the substrate may be associated with high levels of background signal. This may arise because of non-specific antibody binding.

An element may comprise a detectable label. The detectable label may be a label that can be detected visually, by way of the label's optical properties. A detectable label may be a fluorescent label. Such a label may be detected using fluorescent techniques, e.g. fluorescent microscopy. Thus, in a particularly preferred embodiment, a detectable label is a fluorophore (e.g. a fluorescent dye) or a fluorescent polypeptide. A fluorescent dye may be a HiLyte fluorescent dye (commercially available from AnaSpec), an AlexaFluor (commercially available from Thermo Fisher), an Atto (commercially available from Sigma-Aldrich), a Quantum Dot commercially available from Sigma-Aldrich), or a Janelia Fluor dyes (available from Janelia, US). A fluorescent polypeptide may be a cyan fluorescent protein (CFP), a yellow fluorescent protein (YFP), a green fluorescent protein (GFP), or a red fluorescent protein (RFP).

In one embodiment, a first module of the element is a fluorescent label (e.g. a fluorescent polypeptide) and a second module of the element is a different fluorescent label (e.g. a different fluorescent polypeptide). For example, a first module of the element may be a YFP and a second module of the element may be a CFP. The fluorescent labels may be carefully selected such that they are capable of functioning as fluorescence resonance energy transfer (FRET) donor and acceptor pairs. Thus, in one embodiment, when the cleavable substrate is intact and has not been cleaved by a clostridial neurotoxin, the fluorescent labels are in close proximity and FRET occurs, and when the cleavable substrate has been cleaved by a clostridial neurotoxin, the fluorescent labels are no longer in close proximity and FRET does not occur. Measuring a change in the FRET signal (e.g. fluorescent properties of the cleavable substrate) may therefore indicate whether or not the cleavable substrate has been cleaved. Suitable cleavable substrates and associated FRET methodology is taught in EP 2332959 A2, which is incorporated herein by reference.

In a preferred embodiment, the element comprises a luciferase. The term “luciferase” as used herein refers to an enzyme that catalyses a bioluminescent reaction, e.g., by catalysing the oxidation of luciferin, emitting light and releasing oxyluciferin. A luciferase may be naturally-occurring or engineered. A “functional” luciferase as used herein is a luciferase that is capable of catalysing a reaction in the presence of a suitable substrate.

Examples of luciferases include Nanoluc (SEQ ID NO: 1), firefly luciferase (e.g. Photinus pyralis luciferase), bacterial luciferase (e.g. Vibrio fischeri or Vibrio harveyi luciferase), sea pansy luciferase (e.g. Renilla reniformis luciferase), dinoflagellate luciferase, Gaussia luciferase, and copepod luciferase. Other suitable luciferases are described U.S. Pat. No. 8,557,970, which is incorporated herein by reference.

Preferably, a luciferase comprises a polypeptide sequence having at least 70% sequence identity to SEQ ID NO: 1. In one embodiment, a luciferase may comprise a polypeptide sequence having at least 80%, 85%, 90%, 95%, 99%, or 99.9% sequence identity to SEQ ID NO: 1. Preferably, a luciferase may comprise SEQ ID NO: 1. Preferably, a luciferase consists of a polypeptide sequence having at least 70% sequence identity to SEQ ID NO: 1. In one embodiment, a luciferase may consist of a polypeptide sequence having at least 80%, 85%, 90%, 95%, 99%, or 99.9% sequence identity to SEQ ID NO: 1. Preferably, a luciferase may consist of SEQ ID NO: 1.

The luciferase may be present as two separate modules of the element for indicating the presence or absence of cleavage of the cleavable substrate at the clostridial neurotoxin cleavage site. A first module of the element may be a first luciferase domain and a second module of the element may be a second luciferase domain.

Thus, in a particularly preferred embodiment, a cleavable substrate is a single-chain polypeptide comprising: (i) a first luciferase domain; (ii) a linker comprising a clostridial neurotoxin cleavage site; and (iii) a second luciferase domain. In one embodiment: (i) when the linker is intact (i.e., wherein the clostridial neurotoxin cleavage site has not been cleaved by a clostridial neurotoxin), the linker functionally joins the first and second luciferase domains, thereby providing a functional luciferase (i.e. the cleavable substrate is capable of luciferase activity); and (ii) when the linker has been cleaved (i.e., wherein the clostridial neurotoxin cleavage site has been cleaved by a clostridial neurotoxin), the linker no longer functionally joins the first and second luciferase domains, resulting in a loss of luciferase activity (i.e. the cleavable substrate is not capable of luciferase activity). The term “single-chain” when used in the context of the cleavable substrate may refer to a single polypeptide molecule having a series of amino acid residues, connected to each other by peptide bonds between the alpha-amino and carboxy groups of adjacent residues. In other words, each recited element of the single-chain polypeptide may be connected to the other element(s) by means of a peptide bond.

The term “functionally join” as used in the context of first and second luciferase domains refers to joining the first and second luciferase domains in such a way that any intervening sequence does not prevent the two fragments from forming an active and functional tertiary structure. The first and second luciferase domains when functionally joined exhibit luciferase activity, e.g. as they would in the absence of the linker.

The first luciferase domain is preferably N-terminal to the linker comprising the clostridial neurotoxin cleavage site. Preferably, the first luciferase domain is the most N-terminal element of the cleavable substrate. A first luciferase domain may comprise a polypeptide sequence having at least 70% sequence identity to SEQ ID NO: 2. In one embodiment, a first luciferase domain may comprise a polypeptide sequence having at least 80%, 85%, 90%, 95%, 99%, or 99.9% sequence identity to SEQ ID NO: 2. Preferably, a first luciferase domain may comprise SEQ ID NO: 2. A first luciferase domain may consist of a polypeptide sequence having at least 70% sequence identity to SEQ ID NO: 2. In one embodiment, a first luciferase domain may consist of a polypeptide sequence having at least 80%, 85%, 90%, 95%, 99%, or 99.9% sequence identity to SEQ ID NO: 2. Preferably, a first luciferase domain may consist of SEQ ID NO: 2.

The second luciferase domain is preferably C-terminal to the linker comprising the clostridial neurotoxin cleavage site. Preferably, the second luciferase domain is the most C-terminal element of the cleavable substrate. A second luciferase domain may comprise a polypeptide sequence having at least 70% sequence identity to SEQ ID NO: 3. In one embodiment, a second luciferase domain may comprise a polypeptide sequence having at least 80%, 85%, 90%, 95%, 99%, or 99.9% sequence identity to SEQ ID NO: 3. Preferably, a second luciferase domain may comprise SEQ ID NO: 3. A second luciferase domain may consist of a polypeptide sequence having at least 70% sequence identity to SEQ ID NO: 3. In one embodiment, a second luciferase domain may consist of a polypeptide sequence having at least 80%, 85%, 90%, 95%, 99%, or 99.9% sequence identity to SEQ ID NO: 3. Preferably, a second luciferase domain may consist of SEQ ID NO: 3.

In some embodiments, the linker may consist of a clostridial neurotoxin cleavage site. However, it is preferred that the linker further comprises one or more spacers. Most preferably the linker comprises (even more preferably consists of) a first spacer N-terminal to the clostridial neurotoxin cleavage site and a second spacer C-terminal to the clostridial neurotoxin cleavage site. Where more than one spacer is present, the spacers may have the same or different polypeptide sequences (preferably the same polypeptide sequences).

It is well-within the skilled person's capability to select an appropriate spacer sequence and size. A spacer may be of any suitable length, such as 3-20, 2-15, 5-15 or 4-8 amino acids in length. A spacer may comprise (or consist of) glycine and serine residues. In particular, a spacer may comprise a polypeptide sequence having at least 70% sequence identity to SEQ ID NO: 39. In one embodiment, a spacer may comprise a polypeptide sequence having at least 80%, 85%, 90%, 95%, 99%, or 99.9% sequence identity to SEQ ID NO: 39. Preferably, a spacer may comprise SEQ ID NO: 39. In particular, a spacer may consist of a polypeptide sequence having at least 70% sequence identity to SEQ ID NO: 39. In one embodiment, a spacer may consist of a polypeptide sequence having at least 80%, 85%, 90%, 95%, 99%, or 99.9% sequence identity to SEQ ID NO: 39. Preferably, a spacer may consist of SEQ ID NO: 39.

Preferably, a cleavable substrate of the invention comprises (from N-terminus to C-terminus): (i) a first luciferase domain; (ii) a first spacer; (iii) a clostridial neurotoxin cleavage site; (iv) a second spacer; and (v) a second luciferase domain. More preferably, a cleavable substrate of the invention may consist of (from N-terminus to C-terminus): (i) a first luciferase domain; (ii) a first spacer; (iii) a clostridial neurotoxin cleavage site; (iv) a second spacer; and (v) a second luciferase domain. In such embodiments, the linker is formed of elements (ii), (iii), and (iv). The first and second spacers preferably have the same polypeptide sequence.

A cleavable substrate may comprise a polypeptide sequence having at least 70% sequence identity to SEQ ID NO: 6. In one embodiment, a cleavable substrate may comprise a polypeptide sequence having at least 80%, 85%, 90%, 95%, 99%, or 99.9% sequence identity to SEQ ID NO: 6. Preferably, a cleavable substrate may comprise SEQ ID NO: 6.

A cleavable substrate may consist of a polypeptide sequence having at least 70% sequence identity to SEQ ID NO: 6. In one embodiment, a cleavable substrate may consist of a polypeptide sequence having at least 80%, 85%, 90%, 95%, 99%, or 99.9% sequence identity to SEQ ID NO: 6. Preferably, a cleavable substrate may consist of SEQ ID NO: 6.

In some instances herein, “cleavable substrates” are referred to. However, this is to indicate that more than one “cleavable substrate” may be present when carrying out the method. It is not intended to necessarily indicate that two or more different types of cleavable substrates are present, although this is encompassed. Thus, the cleavable substrates may be of one type (e.g. all the cleavable substrates comprise: (i) a first luciferase domain; (ii) a linker comprising a SNAP-25 clostridial neurotoxin cleavage site; and (iii) a second luciferase domain) or multiple types (e.g. a portion of the cleavable substrates comprise: (i) a first luciferase domain; (ii) a linker comprising a SNAP-25 clostridial neurotoxin cleavage site; and (iii) a second luciferase domain and a portion of the cleavable substrates comprise: (i) a YFP; (ii) a linker comprising a SNAP-25 clostridial neurotoxin cleavage site; and (iii) a GFP). Preferably, the cleavable substrates are of one type.

A method may comprise determining whether or not cleavable substrates have been cleaved. In particular, a method may comprise determining an amount of cleavable substrates cleaved (preferably in the assay sample) by the L-chain polypeptides, thereby determining the clostridial neurotoxin activity of the composition. The method of making such a determination will depend on the specific nature of the cleavable substrates used in the method. For example, where a cleavable substrate comprises a detectable label, a difference in the detection properties may indicate whether or not cleavable substrates have been cleaved and/or an amount of cleavable substrates that have been cleaved. Said difference may be determined by comparison to a suitable control. For example, determining whether or not cleavable substrates comprising a fluorescent label have been cleaved may comprise comparing fluorescence of cleavable substrates with a control and determining whether there is a difference in fluorescence (e.g. a loss of a FRET signal).

A suitable control may be a negative control. The negative control may be provided by carrying out the method in the same way but wherein the composition tested does not comprise clostridial neurotoxin polypeptides. The negative control may be a negative reference standard. The negative reference standard may correspond to a value that has been either theoretically or experimentally determined and which represents a negative result in a method of the invention. The value may have been determined prior to carrying out a method of the invention or may be determined simultaneously with, or subsequent to, carrying out a method of the invention.

In one embodiment, when the control is a negative control, a difference in a property of the cleavable substrates when compared to the negative control indicates that a composition tested comprises L-chain polypeptides (preferably comprises clostridial neurotoxin polypeptides). A difference in a property of the cleavable substrate when compared to the negative control may indicate that a composition also comprises H-chain polypeptides (or H_{C} or H_{CC} domain polypeptides). In such instances, this may allow for a determination to be made that a composition comprises clostridial neurotoxin polypeptides.

In one embodiment, when the control is a negative control, no difference in a property of the cleavable substrates when compared to the negative control indicates that a composition tested does not comprise L-chain polypeptides (preferably does not comprise clostridial neurotoxin polypeptides). In one embodiment, when the control is a negative control, no difference in a property of the cleavable substrates when compared to the negative control indicates that a composition tested does not comprise L-chain polypeptides and/or does not comprise H-chain polypeptides (or H_{C} or H_{CC} domain polypeptides) (preferably does not comprise clostridial neurotoxin polypeptides). In such instances, this may allow for a determination to made that a composition does not comprise clostridial neurotoxin polypeptides.

In one embodiment, when the control is a negative control, a difference in a property of the cleavable substrates when compared to the negative control indicates that a composition tested comprises active L-chain polypeptides (preferably comprises active clostridial neurotoxin polypeptides). The extent of the difference may be quantified to indicate the number of active L-chain polypeptides (e.g. the number of active clostridial neurotoxin polypeptides). A difference in a property of the cleavable substrates when compared to the negative control may indicate that a composition also comprises H-chain polypeptides (or H_{C} or H_{CC} domain polypeptides) that do not comprise an activity-reducing property. The extent of the difference may be quantified to indicate the number of H-chain polypeptides (or H_{C} or H_{CC} domain polypeptides) that do not comprise an activity-reducing property (e.g. the number of active clostridial neurotoxin polypeptides). The extent of the difference may be quantified to indicate the activity level of the composition comprising clostridial neurotoxin polypeptides.

Preferably, the greater the difference compared to the negative control, the higher the number of active L-chain polypeptides (e.g. the number of active clostridial neurotoxin polypeptides). Preferably, the greater the difference compared to the negative control, the higher the number of H-chain polypeptides (or H_{C} or H_{CC} domain polypeptides) that do not comprise an activity-reducing property. Preferably, the greater the difference compared to the negative control, the higher the activity level of the composition comprising the clostridial neurotoxin polypeptides.

In one embodiment, when the control is a negative control, no difference in a property of the cleavable substrates when compared to the negative control indicates that a composition tested comprises inactive L-chain polypeptides (preferably comprises inactive clostridial neurotoxin polypeptides). In one embodiment, when the control is a negative control, no difference in a property of the cleavable substrates when compared to the negative control indicates that a composition tested comprises inactive L-chain polypeptides and/or comprises H-chain polypeptides (or H_{C} or H_{CC} domain polypeptides) that comprise an activity-reducing property. Such a composition may be considered an inactive composition.

In one embodiment, when the control is a negative control, a difference in a property of the cleavable substrates when compared to the negative control may be employed to indicate an activity level of the composition. Said difference may be used to indicate the number of active L-chain polypeptides (e.g. the number of active clostridial neurotoxin polypeptides) present in a composition. Said difference may be used to indicate the number of H-chain polypeptides (or H_{C} or H_{CC} domain polypeptides) that do not comprise an activity-reducing property present in a composition.

In some embodiments a difference in a property of cleavable substrates when compared to a control may be quantified to indicate an activity level of the composition (e.g. the number of active clostridial neurotoxin polypeptides present in the composition).

A suitable control may be a positive control. The positive control may be provided by carrying out the method in the same way but wherein the composition comprises clostridial neurotoxin polypeptides, preferably associated with a known activity level. The positive control may be a positive reference standard. The positive reference standard may correspond to a value that has been either theoretically or experimentally determined and which represents a positive result in a method of the invention. The positive result may represent an ideal activity level for a composition comprising clostridial neurotoxin polypeptides. Such a value may be used for quality-control purposes. The value may have been determined prior to carrying out a method of the invention or may be determined simultaneously with, or subsequent to, carrying out a method of the invention. Thus, where “a property of the cleavable substrates” or a “luminescence” (e.g. a level of luminescence) is used herein in reference to a control, this may mean “a property of the cleavable substrates” or a “luminescence” (e.g. a level of luminescence) represented by said control.

In one embodiment, when the control is a positive control, no difference in a property of the cleavable substrates when compared to the positive control indicates that a composition tested comprises L-chain polypeptides (preferably comprises clostridial neurotoxin polypeptides). No difference in a property of the cleavable substrate when compared to the positive control may indicate that a composition also comprises H-chain polypeptides (or H_{C} or H_{CC} domain polypeptides). In such instances, this may allow for a determination to be made that a composition comprises clostridial neurotoxin polypeptides.

In one embodiment, when the control is a positive control, no difference in a property of the cleavable substrates when compared to the positive control indicates that a composition tested comprises active L-chain polypeptides (preferably comprises active clostridial neurotoxin polypeptides). No difference in a property of the cleavable substrate when compared to the positive control may indicate that a composition also comprises H-chain polypeptides (or H_{C} or H_{CC} domain polypeptides) that do not comprise an activity-reducing property. Such a composition may be considered an active composition.

In one embodiment, when the control is a positive control, a difference in a property of the cleavable substrates when compared to the positive control may be employed to indicate an activity level of the composition. Said difference may be used to indicate the number of active L-chain polypeptides (e.g. the number of active clostridial neurotoxin polypeptides) present in a composition. Said difference may be used to indicate the number of H-chain polypeptides (or H_{C} or H_{CC} domain polypeptides) that do not comprise an activity-reducing property present in a composition.

In one embodiment, when the control is a positive control, no difference in a property of the cleavable substrates when compared to the positive control may be employed to indicate that the composition has the same activity level as the positive control. This may be used to indicate the number of active L-chain polypeptides (e.g. the number of active clostridial neurotoxin polypeptides) present in a composition. This may be used to indicate the number of H-chain polypeptides (or H_{C} or H_{CC} domain polypeptides) that do not comprise an activity-reducing property present in a composition.

Where the cleavable substrates comprise (i) a first luciferase domain; (ii) a linker comprising a clostridial neurotoxin cleavage site; and (iii) a second luciferase domain, determining whether or not cleavable substrates have been cleaved may be assessed by measuring luciferase activity. This may be achieved by adding a suitable luciferase substrate (preferably adding said luciferase substrate to the assay sample). Preferably, the luciferase substrate is furimazine (2-furanylmethyl-deoxy-coelenterazine). In such instances, a determination of whether or not cleavable substrates have been cleaved can be assessed by measuring luminescence. For example, the cleavable substrates may be contacted with furimazine (preferably in the assay sample) at a concentration of 1-100 μM, preferably 10-50 μM (e.g. 37.5 μM). Luminescence may be measured using any suitable technique, such as an automated plate reader (e.g. a BMG Labtech CLARIOstar plate reader).

Thus, in preferred embodiments, a property of the cleavable substrates may be luciferase activity. A difference in the property of the cleavable substrates may be a difference in the level of luciferase activity. As indicated above, this may be determined by measuring luminescence.

Thus, in one embodiment, when the control is a negative control, a lower level of luminescence when compared to the negative control indicates that a composition tested comprises L-chain polypeptides (preferably comprises clostridial neurotoxin polypeptides). A lower level of luminescence when compared to the negative control may indicate that a composition tested also comprises H-chain polypeptides (or H_{C} or H_{CC} domain polypeptides). In such instances, this may allow for a determination to made that a composition comprises clostridial neurotoxin polypeptides.

In one embodiment, when the control is a negative control, the same luminescence when compared to the negative control indicates that a composition tested does not comprise L-chain polypeptides (preferably does not comprise clostridial neurotoxin polypeptides). In one embodiment, when the control is a negative control, the same luminescence when compared to the negative control indicates that a composition tested does not comprise L-chain polypeptides and/or does not comprise H-chain polypeptides (or H_{C} or H_{CC} domain polypeptides) (preferably does not comprise clostridial neurotoxin polypeptides). In such instances, this may allow for a determination to made that a composition does not comprise clostridial neurotoxin polypeptides.

In one embodiment, when the control is a negative control, a lower level of luminescence when compared to the negative control indicates that a composition tested comprises active L-chain polypeptides (preferably comprises active clostridial neurotoxin polypeptides). The extent by which the level is lower may be quantified to indicate the number of active L-chain polypeptides (e.g. the number of active clostridial neurotoxin polypeptides). A lower level of luminescence when compared to the negative control may indicate that a composition also comprises H-chain polypeptides (or H_{C} or H_{CC} domain polypeptides) that do not comprise an activity-reducing property. The extent by which the level is lower may be quantified to indicate the activity level of the composition comprising clostridial neurotoxin polypeptides. Preferably, the lower the level of luminescence compared to the negative control, the higher the number of active L-chain polypeptides (e.g. the number of active clostridial neurotoxin polypeptides). Preferably, the lower the level of luminescence compared to the negative control, the higher the number of H-chain polypeptides (or H_{C} or H_{CC} domain polypeptides) that do not comprise an activity-reducing property. Preferably, the lower the level of luminescence compared to the negative control, the higher the activity level of the composition comprising clostridial neurotoxin polypeptides.

In one embodiment, when the control is a negative control, the same luminescence when compared to the negative control indicates that a composition tested comprises inactive L-chain polypeptides (preferably comprises inactive clostridial neurotoxin polypeptides). In one embodiment, when the control is a negative control, the same luminescence when compared to the negative control indicates that a composition tested comprises inactive L-chain polypeptides and/or comprises H-chain polypeptides (or H_{C} or H_{CC} domain polypeptides) that comprise an activity-reducing property.

Thus, in one embodiment, when the control is a positive control, the same (or a lower level of) luminescence when compared to the positive control indicates that a composition tested comprises L-chain polypeptides (preferably comprises clostridial neurotoxin polypeptides). The same (or a lower level of) luminescence when compared to the positive control may indicate that a composition also comprises H-chain polypeptides (or H_{C} or H_{CC} domain polypeptides). In such instances, this may allow for a determination to be made that a composition comprises clostridial neurotoxin polypeptides.

In one embodiment, when the control is a positive control, the same (or a lower level of) luminescence when compared to the positive control indicates that a composition tested comprises active L-chain polypeptides (preferably comprises active clostridial neurotoxin polypeptides). The same (or a lower level of) luminescence when compared to the positive control may indicate that a composition also comprises H-chain polypeptides (or H_{C} or H_{CC} domain polypeptides) that do not comprise an activity-reducing property.

The same level of luminescence when compared to the positive control may indicate that the composition tested comprises the same number of active L-chain polypeptides (e.g. active clostridial neurotoxin polypeptides) as the positive control. The same level of luminescence when compared to the positive control may indicate that the composition tested comprises the same number of H-chain polypeptides (or H_{C} or H_{CC} domain polypeptides) that do not comprise an activity-reducing property as the positive control. The same level of luminescence when compared to the positive control may indicate that the composition has the same activity level as the positive control.

A lower level of luminescence when compared to the positive control may indicate that the composition tested comprises a higher number of active L-chain polypeptides (e.g. active clostridial neurotoxin polypeptides) than the positive control. A lower level of luminescence when compared to the positive control may indicate that the composition tested comprises a higher number of H-chain polypeptides (or H_{C} or H_{CC} domain polypeptides) that do not comprise an activity-reducing property than the positive control and/or comprises H-chain polypeptides (or H_{C} or H_{CC} domain polypeptides) that comprise an activity-increasing property. A lower level of luminescence when compared to the positive control may indicate that the composition has a higher activity level than the positive control. The extent by which the level is lower may be quantified to indicate the activity level of the composition comprising clostridial neurotoxin polypeptides. Preferably, the lower the level of luminescence compared to the positive control, the higher the number of active L-chain polypeptides (e.g. the number of active clostridial neurotoxin polypeptides). Preferably, the lower the level of luminescence compared to the positive control, the higher the number of H-chain polypeptides (or H_{C} or H_{CC} domain polypeptides) that do not comprise an activity-reducing property. Preferably, the lower the level of luminescence compared to the positive control, the greater the effect of an activity-increasing property of the H-chain polypeptides (or H_{C} or H_{CC} domain polypeptides). Preferably, the lower the level of luminescence compared to the positive control, the higher the activity level of the composition comprising clostridial neurotoxin polypeptides.

In one embodiment, when the control is a positive control, a higher level of luminescence when compared to the positive control indicates that a composition tested does not comprise L-chain polypeptides (preferably comprises clostridial neurotoxin polypeptides). In one embodiment, when the control is a positive control, a higher level of luminescence when compared to the positive control indicates that a composition tested does not comprise L-chain polypeptides (preferably comprises clostridial neurotoxin polypeptides) and/or does not comprise H-chain polypeptides (or H_{C} or H_{CC} domain polypeptides). In such instances, this may allow for a determination to made that a composition does not comprise clostridial neurotoxin polypeptides. To make a more definitive determination, a comparison may also be made to a negative control, as described herein.

In one embodiment, when the control is a positive control, a higher level of luminescence when compared to the positive control indicates that a composition tested comprises inactive L-chain polypeptides (preferably comprises active clostridial neurotoxin polypeptides). In one embodiment, when the control is a positive control, a higher level of luminescence when compared to the positive control indicates that a composition tested comprises inactive L-chain polypeptides (preferably comprises active clostridial neurotoxin polypeptides) and/or comprises H-chain polypeptides (or H_{C} or H_{CC} domain polypeptides) comprising an activity-reducing property.

A higher level of luminescence when compared to the positive control may indicate that the composition tested comprises fewer active L-chain polypeptides (e.g. active clostridial neurotoxin polypeptides) than the positive control. A higher level of luminescence when compared to the positive control may indicate that the composition tested comprises fewer H-chain polypeptides (or H_{C} or H_{CC} domain polypeptides) that do not comprise an activity-reducing property than the positive control. A higher level of luminescence when compared to the positive control may indicate that the composition has a lower activity level than the positive control. The extent by which the level is higher may be quantified to indicate the activity level of the composition comprising clostridial neurotoxin polypeptides. Preferably, the higher the level of luminescence compared to the positive control, the lower the number of active L-chain polypeptides (e.g. the number of active clostridial neurotoxin polypeptides). Preferably, the higher the level of luminescence compared to the positive control, the lower the number of H-chain polypeptides (or H_{C} or H_{CC} domain polypeptides) that do not comprise an activity-reducing property. Preferably, the higher the level of luminescence compared to the positive control, the lower the activity level of the composition comprising clostridial neurotoxin polypeptides.

The term “different” (and associated terms such as “change”, “changed”, and “difference”, and synonyms thereof) as used herein may mean a difference that is substantially different to a comparator (e.g. a control as described herein). A difference (and associated terms such as “change”, “changed”, and “difference”, and synonyms thereof) may mean a statistically-significant difference when compared to a comparator (e.g. a control as described herein). A “substantial difference” may be a difference of at least 5%, 10%, 15%, 20%, 25% or 30% when compared to a comparator (e.g. a control as described herein). The term “no difference” (and associated terms such as “unchanged” and “the same”, and synonyms thereof) may mean that there is no substantial difference when compared to a comparator (e.g. a control as described herein). No difference (and associated terms such as “unchanged” and “the same”, and synonyms thereof) may mean that there is no statistically-significant difference when compared to a comparator (e.g. a control as described herein). The term “lower” (and associated terms such as “less than”) as used herein may mean at least 10%, 25%, 20%, 50%, 75%, 100%, 150%, or 200% lower when compared to a comparator (e.g. a control as described herein). The term “lower” (and associated terms such as “less than”) may mean statistically-significantly lower when compared to a comparator (e.g. a control as described herein). The term “higher” (and associated terms such as “higher than”) as used herein may mean at least 10%, 25%, 20%, 50%, 75%, 100%, 150%, or 200% higher when compared to a comparator (e.g. a control as described herein). The term “higher” (and associated terms such as “higher than”) may mean statistically-significantly higher when compared to a comparator (e.g. a control as described herein).

A final step of a method of the invention may comprise assigning a measured activity value. Said measured activity value may be a luciferase activity value (e.g. determined by luminescence). Preferably, said measured activity value is a measured luminescence value. In some embodiments, said measured activity value (e.g. luminescence value) is compared to a control value. A method of the invention may be used to determine a potency value (EC₅₀) of a composition comprising clostridial neurotoxin polypeptides. In order to obtain said potency value, the method may be carried out using at least a second third and/or fourth composition comprising a different concentration of the clostridial neurotoxin polypeptides, and determining an activity value for the at least second, third, and/or fourth compositions and determining a potency value (EC₅₀) of the composition by comparing the measured activity value (for the first composition) with the measured luciferase activity value(s) for the at least second, third, and/or fourth compositions.

The “EC₅₀” may refer to a clostridial neurotoxin (e.g. amount or concentration thereof in a composition) which induces a response halfway between the baseline and maximum after some specified exposure time. It is commonly used as a measure of potency. The EC₅₀ of a graded dose response curve may represent the concentration of a clostridial neurotoxin where 50% of its maximal effect is observed.

In some instances herein, “clostridial neurotoxin polypeptides” and “light-chain polypeptides” (respectively) are referred to. However, this is to indicate that more than one clostridial neurotoxin polypeptide or light-chain polypeptide (respectively) may be present (e.g. in a composition) when carrying out the method. It is not intended to necessarily indicate that two or more different types of clostridial neurotoxin polypeptides or light-chain polypeptides (respectively) are present, although this is encompassed. Thus, the clostridial neurotoxin polypeptides or light-chain polypeptides (respectively) may be of one type (e.g. all the clostridial neurotoxin polypeptides are BoNT/A polypeptides or all the light-chain polypeptides are BoNT/A light-chain polypeptides [respectively]) or multiple types (e.g. a portion of the clostridial neurotoxin polypeptides are BoNT/B polypeptides and a portion are BoNT/A polypeptides or a portion the light-chain polypeptides are BoNT/A light-chain polypeptides and a portion are BoNT/D light-chain polypeptides [respectively]). Preferably, the clostridial neurotoxin polypeptides are of one type. Preferably, the light-chain polypeptides are of one type. The above applies analogously to some instances herein where “botulinum neurotoxin polypeptides” are referred to.

A clostridial neurotoxin according to the invention may comprise a botulinum neurotoxin or a tetanus neurotoxin (TeNT) H_{CC} domain. A clostridial neurotoxin of the invention may comprise a BoNT/A H_{CC} domain, a BoNT/B H_{CC} domain, a BoNT/C1 H_{CC} domain, a BoNT/D H_{CC} domain, a BoNT/E H_{CC} domain, a BoNT/F H_{CC} domain, a BoNT/G H_{CC} domain, a BoNT/X H_{CC} domain, or TeNT H_{CC} domain. Preferably, a clostridial neurotoxin of the invention comprises a BoNT/B H_{CC} domain or a BoNT/A H_{CC} domain, more preferably a BoNT/B H_{CC} domain.

A clostridial neurotoxin according to the invention may comprise a botulinum neurotoxin or a tetanus neurotoxin (TeNT) H_{C} domain. A clostridial neurotoxin of the invention may comprise a BoNT/A H_{C} domain, a BoNT/B H_{C} domain, a BoNT/C1 H_{C} domain, a BoNT/D H_{C} domain, a BoNT/E H_{C} domain, a BoNT/F H_{C} domain, a BoNT/G H_{C} domain, a BoNT/X H_{C} domain, or TeNT H_{C} domain. Preferably, a clostridial neurotoxin of the invention comprises a BoNT/B H_{C} domain or a BoNT/A H_{C} domain, more preferably a BoNT/B H_{C} domain.

The term “clostridial neurotoxin” embraces toxins produced by C. botulinum (botulinum neurotoxin serotypes A, B, C1, D, E, F, G, and X), C. tetani (tetanus neurotoxin), C. butyricum (botulinum neurotoxin serotype E), and C. baratii (botulinum neurotoxin serotype F). A reference BoNT/A sequence is shown as SEQ ID NO: 12. A reference BoNT/B sequence is shown as SEQ ID NO: 13. A reference BoNT/C1 (also referred to as BoNT/C herein) sequence is shown as SEQ ID NO: 41. A reference BoNT/D sequence is shown as SEQ ID NO: 42. A reference BoNT/E sequence is shown as SEQ ID NO: 43. A reference BoNT/F sequence is shown as SEQ ID NO: 44. A reference BoNT/G sequence is shown as SEQ ID NO: 45. A reference TeNT sequence is shown as SEQ ID NO: 46. A reference BoNT/X sequence is shown as SEQ ID NO: 47. The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO2244.1), which cleaves VAMP2 and SNAP25, and the Chryseobacterium pipero encoded toxin (NCBI Ref.Seq: WP_034687872.1).

Thus, a clostridial neurotoxin may be selected from BoNT/A, BoNT/B, BoNT/C, BoNT/D, BoNT/E, BoNT/F, BoNT/G, BoNT/X, and TeNT (tetanus neurotoxin). Thus, a composition of the invention may comprise BoNT/A, BoNT/B, BoNT/C, BoNT/D, BoNT/E, BoNT/F, BoNT/G, BoNT/X, or TeNT. Therefore, the clostridial neurotoxin polypeptides may be BoNT/A polypeptides, BoNT/B polypeptides, BoNT/C polypeptides, BoNT/D polypeptides, BoNT/E polypeptides, BoNT/F polypeptides, BoNT/G polypeptides, BoNT/X polypeptides, or TeNT polypeptides. Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X. Thus, a composition of the invention may comprise BoNT/A, BoNT/B, BoNT/C, BoNT/D, BoNT/E, BoNT/F, BoNT/G, or BoNT/X. Therefore, the clostridial neurotoxin polypeptides may be BoNT/A polypeptides, BoNT/B polypeptides, BoNT/C polypeptides, BoNT/D polypeptides, BoNT/E polypeptides, BoNT/F polypeptides, BoNT/G polypeptides, or BoNT/X polypeptides.

Clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain). Botulinum neurotoxin (BoNT) is produced by C. botulinum in the form of a large protein complex, consisting of BoNT itself complexed to a number of accessory proteins. There are at present eight different classes of botulinum neurotoxin, namely: botulinum neurotoxin serotypes A, B, C1, D, E, F, G, and X all of which share similar structures and modes of action. Different BoNT serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

BoNTs are absorbed in the gastrointestinal tract, and, after entering the general circulation, bind to the presynaptic membrane of cholinergic nerve terminals and prevent the release of their neurotransmitter acetylcholine. BoNT/B, BoNT/D, BoNT/F and BoNT/G cleave synaptobrevin/vesicle-associated membrane protein (VAMP); BoNT/C1, BoNT/A and BoNT/E cleave the synaptosomal-associated protein of 25 kDa (SNAP-25); and BoNT/C1 cleaves syntaxin. BoNT/X has been found to cleave SNAP-25, VAMP1, VAMP2, VAMP3, VAMP4, VAMP5, Ykt6, and syntaxin 1. Tetanus toxin is produced in a single serotype by C. tetani. C. butyricum produces BoNT/E, while C. baratii produces BoNT/F.

### Examples of L-Chain Reference Sequences Include:

Botulinum type A neurotoxin: amino acid residues 1-448
Botulinum type B neurotoxin: amino acid residues 1-440
Botulinum type C1 neurotoxin: amino acid residues 1-441
Botulinum type D neurotoxin: amino acid residues 1-445
Botulinum type E neurotoxin: amino acid residues 1-422
Botulinum type F neurotoxin: amino acid residues 1-439
Botulinum type G neurotoxin: amino acid residues 1-441
Tetanus neurotoxin: amino acid residues 1-457

For recently-identified BoNT/X, the L-chain has been reported as corresponding to amino acids 1-439 thereof, with the L-chain boundary potentially varying by approximately 25 amino acids (e.g. 1-414 or 1-464).

A BoNT/A L-chain may comprise a polypeptide sequence having at least 70%, 80%, 85%, 90%, 95%, 99%, 99.9%, or 100% to amino acid residues 1-448 of SEQ ID NO: 12. A BoNT/B L-chain may comprise a polypeptide sequence having at least 70%, 80%, 85%, 90%, 95%, 99%, 99.9%, or 100% to amino acid residues 1-440 of SEQ ID NO: 13. A BoNT/C1 L-chain may comprise a polypeptide sequence having at least 70%, 80%, 85%, 90%, 95%, 99%, 99.9%, or 100% to amino acid residues 1-441 of SEQ ID NO: 41. A BoNT/D L-chain may comprise a polypeptide sequence having at least 70%, 80%, 85%, 90%, 95%, 99%, 99.9%, or 100% to amino acid residues 1-445 of SEQ ID NO: 42. A BoNT/E L-chain may comprise a polypeptide sequence having at least 70%, 80%, 85%, 90%, 95%, 99%, 99.9%, or 100% to amino acid residues 1-422 of SEQ ID NO: 43. A BoNT/F L-chain may comprise a polypeptide sequence having at least 70%, 80%, 85%, 90%, 95%, 99%, 99.9%, or 100% to amino acid residues 1-439 of SEQ ID NO: 44. A BoNT/G L-chain may comprise a polypeptide sequence having at least 70%, 80%, 85%, 90%, 95%, 99%, 99.9%, or 100% to amino acid residues 1-441 of SEQ ID NO: 45. A BoNT/X L-chain may comprise a polypeptide sequence having at least 70%, 80%, 85%, 90%, 95%, 99%, 99.9%, or 100% to amino acid residues 1-439 of SEQ ID NO: 47. A TeNT L-chain may comprise a polypeptide sequence having at least 70%, 80%, 85%, 90%, 95%, 99%, 99.9%, or 100% to amino acid residues 1-457 of SEQ ID NO: 46.

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441
Botulinum type C1 neurotoxin: amino acid residues M1-K449
Botulinum type D neurotoxin: amino acid residues M1-R445
Botulinum type E neurotoxin: amino acid residues M1-R422
Botulinum type F neurotoxin: amino acid residues M1-K439
Botulinum type G neurotoxin: amino acid residues M1-K446
Tetanus neurotoxin: amino acid residues M1-A457

The translocation domain is a fragment of the H-chain of a clostridial neurotoxin approximately equivalent to the amino-terminal half of the H-chain, or the domain corresponding to that fragment in the intact H-chain. In one embodiment the H_{C} function of the H-chain may be removed by deletion of the H_{C} amino acid sequence (either at the DNA synthesis level, or at the post-synthesis level by nuclease or protease treatment). Alternatively, the H_{C} function may be inactivated by chemical or biological treatment. Thus, in some embodiments the H-chain may be incapable of binding to the Binding Site on a target cell to which native clostridial neurotoxin (i.e. holotoxin) binds.

### Examples of Suitable (Reference) Translocation Domains Include:

Botulinum type A neurotoxin-amino acid residues (449-871)
Botulinum type B neurotoxin-amino acid residues (441-858)
Botulinum type C neurotoxin-amino acid residues (442-866)
Botulinum type D neurotoxin-amino acid residues (446-862)
Botulinum type E neurotoxin-amino acid residues (423-845)
Botulinum type F neurotoxin-amino acid residues (440-864)
Botulinum type G neurotoxin-amino acid residues (442-863)
Tetanus neurotoxin-amino acid residues (458-879)

A BoNT/A H_{N} domain may comprise a polypeptide sequence having at least 70%, 80%, 85%, 90%, 95%, 99%, 99.9%, or 100% to amino acid residues 449-871 of SEQ ID NO: 12. A BoNT/B H_{N} domain may comprise a polypeptide sequence having at least 70%, 80%, 85%, 90%, 95%, 99%, 99.9%, or 100% to amino acid residues 441-858 of SEQ ID NO: 13. A BoNT/C1 H_{N} domain may comprise a polypeptide sequence having at least 70%, 80%, 85%, 90%, 95%, 99%, 99.9%, or 100% to amino acid residues 442-866 of SEQ ID NO: 41. A BoNT/D H_{N} domain may comprise a polypeptide sequence having at least 70%, 80%, 85%, 90%, 95%, 99%, 99.9%, or 100% to amino acid residues 446-862 of SEQ ID NO: 42. A BoNT/E H_{N} domain may comprise a polypeptide sequence having at least 70%, 80%, 85%, 90%, 95%, 99%, 99.9%, or 100% to amino acid residues 423-845 of SEQ ID NO: 43. A BoNT/F H_{N} domain may comprise a polypeptide sequence having at least 70%, 80%, 85%, 90%, 95%, 99%, 99.9%, or 100% to amino acid residues 440-864 of SEQ ID NO: 44. A BoNT/G H_{N} domain may comprise a polypeptide sequence having at least 70%, 80%, 85%, 90%, 95%, 99%, 99.9%, or 100% to amino acid residues 442-863 of SEQ ID NO: 45. A BoNT/X H_{N} domain may comprise a polypeptide sequence having at least 70%, 80%, 85%, 90%, 95%, 99%, 99.9%, or 100% to amino acid residues 440-892 of SEQ ID NO: 47. A TeNT H_{N} domain may comprise a polypeptide sequence having at least 70%, 80%, 85%, 90%, 95%, 99%, 99.9%, or 100% to amino acid residues 458-879 of SEQ ID NO: 46.

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin-amino acid residues (A449-K871)
Botulinum type B neurotoxin-amino acid residues (A442-S858)
Botulinum type C neurotoxin-amino acid residues (T450-N866)
Botulinum type D neurotoxin-amino acid residues (D446-N862)
Botulinum type E neurotoxin-amino acid residues (K423-K845)
Botulinum type F neurotoxin-amino acid residues (A440-K864)
Botulinum type G neurotoxin-amino acid residues (S447-S863)
Tetanus neurotoxin-amino acid residues (S458-V879)

In the context of the present invention, a variety of clostridial neurotoxin H_{N} regions comprising a translocation domain can be useful in aspects of the present invention. The H_{N} regions from the heavy chains of clostridial neurotoxins are approximately 410-430 amino acids in length and comprise a translocation domain. Research has shown that the entire length of a H_{N} region from a clostridial neurotoxin heavy chain is not necessary for the translocating activity of the translocation domain. Thus, aspects of this embodiment can include clostridial neurotoxin H_{N} regions comprising a translocation domain having a length of, for example, at least 350 amino acids, at least 375 amino acids, at least 400 amino acids and at least 425 amino acids. Other aspects of this embodiment can include clostridial neurotoxin H_{N} regions comprising a translocation domain having a length of, for example, at most 350 amino acids, at most 375 amino acids, at most 400 amino acids and at most 425 amino acids.

For further details on the genetic basis of toxin production in Clostridium botulinum and C. tetani, see Henderson et al (1997) in The Clostridia: Molecular Biology and Pathogenesis, Academic press.

The term H_{N} embraces naturally-occurring neurotoxin H_{N} portions, and modified H_{N} portions having amino acid sequences that do not occur in nature and/or synthetic amino acid residues. In one embodiment said modified H_{N} portions still demonstrate the above-mentioned translocation function.

### Examples of Clostridial Neurotoxin Receptor Binding Domain (H_{C}) Reference Sequences Include:

BoNT/A-N872-L1296
BoNT/B-E859-E1291
BoNT/C1-N867-E1291
BoNT/D-S863-E1276
BoNT/E-R846-K1252
BoNT/F-K865-E1274
BoNT/G-N864-E1297
TeNT-1880-D1315

For recently-identified BoNT/X, the H_{C} domain has been reported as corresponding to amino acids 893-1306 thereof, with the domain boundary potentially varying by approximately 25 amino acids (e.g. 868-1306 or 918-1306).

A BoNT/A H_{C} domain may comprise a polypeptide sequence having at least 70%, 80%, 85%, 90%, 95%, 99%, 99.9%, or 100% to amino acid residues 872-1296 of SEQ ID NO: 12. A BoNT/B H_{C} domain may comprise a polypeptide sequence having at least 70%, 80%, 85%, 90%, 95%, 99%, 99.9%, or 100% to amino acid residues 859-1291 of SEQ ID NO: 13. A BoNT/C1 H_{C} domain may comprise a polypeptide sequence having at least 70%, 80%, 85%, 90%, 95%, 99%, 99.9%, or 100% to amino acid residues 867-1291 of SEQ ID NO: 41. A BoNT/D H_{C} domain may comprise a polypeptide sequence having at least 70%, 80%, 85%, 90%, 95%, 99%, 99.9%, or 100% to amino acid residues 863-1276 of SEQ ID NO: 42. A BoNT/E H_{C} domain may comprise a polypeptide sequence having at least 70%, 80%, 85%, 90%, 95%, 99%, 99.9%, or 100% to amino acid residues 846-1252 of SEQ ID NO: 43. A BoNT/F H_{C} domain may comprise a polypeptide sequence having at least 70%, 80%, 85%, 90%, 95%, 99%, 99.9%, or 100% to amino acid residues 865-1274 of SEQ ID NO: 44. A BoNT/G H_{C} domain may comprise a polypeptide sequence having at least 70%, 80%, 85%, 90%, 95%, 99%, 99.9%, or 100% to amino acid residues 864-1297 of SEQ ID NO: 45. A BoNT/X H_{C} domain may comprise a polypeptide sequence having at least 70%, 80%, 85%, 90%, 95%, 99%, 99.9%, or 100% to amino acid residues 893-1306 of SEQ ID NO: 47. A TeNT H_{C} domain may comprise a polypeptide sequence having at least 70%, 80%, 85%, 90%, 95%, 99%, 99.9%, or 100% to amino acid residues 880-1315 of SEQ ID NO: 46.

A clostridial neurotoxin H-chain (e.g. the H_{C} domain portion) may further comprise a translocation facilitating domain (or a fragment thereof may be translocation facilitating domain fragment). Said domain facilitates delivery of the L-chain into the cytosol of the target cell and are described, for example, in WO 08/008803 and WO 08/008805, each of which is herein incorporated by reference thereto.

By way of example, a translocation facilitating domain may comprise a clostridial neurotoxin H_{CN} domain or a fragment or variant thereof. In more detail, a clostridial neurotoxin H_{CN} translocation facilitating domain may have a length of at least 200 amino acids, at least 225 amino acids, at least 250 amino acids, at least 275 amino acids. In this regard, a clostridial neurotoxin H_{CN} translocation facilitating domain preferably has a length of at most 200 amino acids, at most 225 amino acids, at most 250 amino acids, or at most 275 amino acids. Specific (reference) examples include:
Botulinum type A neurotoxin-amino acid residues (872-1110)
Botulinum type B neurotoxin-amino acid residues (859-1097)
Botulinum type C neurotoxin-amino acid residues (867-1111)
Botulinum type D neurotoxin-amino acid residues (863-1098)
Botulinum type E neurotoxin-amino acid residues (846-1085)
Botulinum type F neurotoxin-amino acid residues (865-1105)
Botulinum type G neurotoxin-amino acid residues (864-1105)
Tetanus neurotoxin-amino acid residues (880-1127)

The above sequence positions may vary a little according to serotype/sub-type, and further examples of suitable (reference) clostridial neurotoxin H_{CN} domains include:
Botulinum type A neurotoxin-amino acid residues (874-1110)
Botulinum type B neurotoxin-amino acid residues (861-1097)
Botulinum type C neurotoxin-amino acid residues (869-1111)
Botulinum type D neurotoxin-amino acid residues (865-1098)
Botulinum type E neurotoxin-amino acid residues (848-1085)
Botulinum type F neurotoxin-amino acid residues (867-1105)
Botulinum type G neurotoxin-amino acid residues (866-1105)
Tetanus neurotoxin-amino acid residues (882-1127)

Any of the above-described facilitating domains may be combined with any of the previously described translocation domain peptides that are suitable for use in the present invention. Thus, by way of example, a non-clostridial facilitating domain may be combined with a non-clostridial translocation domain peptide or with clostridial translocation domain peptide. Alternatively, a clostridial neurotoxin H_{CN} translocation facilitating domain may be combined with a non-clostridial translocation domain peptide. Alternatively, a clostridial neurotoxin H_{CN} facilitating domain may be combined with a clostridial translocation domain peptide, examples of which include:
Botulinum type A neurotoxin-amino acid residues (449-1110)
Botulinum type B neurotoxin-amino acid residues (442-1097)
Botulinum type C neurotoxin-amino acid residues (450-1111)
Botulinum type D neurotoxin-amino acid residues (446-1098)
Botulinum type E neurotoxin-amino acid residues (423-1085)
Botulinum type F neurotoxin-amino acid residues (440-1105)
Botulinum type G neurotoxin-amino acid residues (447-1105)
Tetanus neurotoxin-amino acid residues (458-1127)

The H_{C} peptide of a native clostridial neurotoxin comprises approximately 400-440 amino acid residues, and consists of two functionally distinct domains of approximately 25 kDa each, namely the N-terminal region (commonly referred to as the H_{CN} peptide or domain) and the C-terminal region (commonly referred to as the H_{CC} peptide or domain). This fact is confirmed by the following publications, each of which is herein incorporated in its entirety by reference thereto: Umland T C (1997) Nat. Struct. Biol. 4:788-792; Herreros J (2000) Biochem. J. 347: 199-204; Halpern J (1993) J. Biol. Chem. 268:15, pp. 11188-11192; Rummel A (2007) PNAS 104:359-364; Lacey D B (1998) Nat. Struct. Biol. 5:898-902; Knapp (1998) Am. Cryst. Assoc. Abstract Papers 25:90; Swaminathan and Eswaramoorthy (2000) Nat. Struct. Biol. 7:1751-1759; and Rummel A (2004) Mol. Microbiol. 51 (3), 631-643. Moreover, it has been well documented that the C-terminal region (H_{CC}), which constitutes the C-terminal 160-200 amino acid residues, is responsible for binding of a clostridial neurotoxin to its natural cell receptors, namely to nerve terminals at the neuromuscular junction—this fact is also confirmed by the above publications. Thus, reference throughout this specification to a clostridial heavy-chain lacking a functional heavy chain H_{C} peptide (or domain) such that the heavy-chain is incapable of binding to cell surface receptors to which a native clostridial neurotoxin binds means that the clostridial heavy-chain simply lacks a functional H_{CC} peptide. In other words, the H_{CC} peptide region may be either partially or wholly deleted, or otherwise modified (e.g. through conventional chemical or proteolytic treatment) to reduce its native binding ability for nerve terminals at the neuromuscular junction.

### H_{CC} Reference Sequences are Presented Below:

Botulinum type A neurotoxin-amino acid residues (Y1111-L1296)
Botulinum type B neurotoxin-amino acid residues (Y1098-E1291)
Botulinum type C neurotoxin-amino acid residues (Y1112-E1291)
Botulinum type D neurotoxin-amino acid residues (Y1099-E1276)
Botulinum type E neurotoxin-amino acid residues (Y1086-K1252)
Botulinum type F neurotoxin-amino acid residues (Y1106-E1274)
Botulinum type G neurotoxin-amino acid residues (Y1106-E1297)
Tetanus neurotoxin-amino acid residues (Y1128-D1315).

The above-identified reference sequences should be considered a guide as slight variations may occur according to sub-serotypes.

A BoNT/A H_{CC} domain may comprise a polypeptide sequence having at least 70%, 80%, 85%, 90%, 95%, 99%, 99.9%, or 100% to amino acid residues 1111-1296 of SEQ ID NO: 12. A BoNT/B H_{CC} domain may comprise a polypeptide sequence having at least 70%, 80%, 85%, 90%, 95%, 99%, 99.9%, or 100% to amino acid residues 1098-1291 of SEQ ID NO: 13. A BoNT/C1 H_{CC} domain may comprise a polypeptide sequence having at least 70%, 80%, 85%, 90%, 95%, 99%, 99.9%, or 100% to amino acid residues 1112-1291 of SEQ ID NO: 41. A BoNT/D H_{CC} domain may comprise a polypeptide sequence having at least 70%, 80%, 85%, 90%, 95%, 99%, 99.9%, or 100% to amino acid residues 1099-1276 of SEQ ID NO: 42. A BoNT/E H_{CC} domain may comprise a polypeptide sequence having at least 70%, 80%, 85%, 90%, 95%, 99%, 99.9%, or 100% to amino acid residues 1086-1252 of SEQ ID NO: 43. A BoNT/F H_{CC} domain may comprise a polypeptide sequence having at least 70%, 80%, 85%, 90%, 95%, 99%, 99.9%, or 100% to amino acid residues 1106-1274 of SEQ ID NO: 44. A BoNT/G H_{CC} domain may comprise a polypeptide sequence having at least 70%, 80%, 85%, 90%, 95%, 99%, 99.9%, or 100% to amino acid residues 1106-1297 of SEQ ID NO: 45. A TeNT H_{CC} domain may comprise a polypeptide sequence having at least 70%, 80%, 85%, 90%, 95%, 99%, 99.9%, or 100% to amino acid residues 1128-1315 of SEQ ID NO: 46.

The term “clostridial neurotoxin” is also intended to embrace modified clostridial neurotoxins and derivatives thereof, including but not limited to those described below. A modified clostridial neurotoxin or derivative may contain one or more amino acids that has been modified as compared to the native (unmodified) form of the clostridial neurotoxin, or may contain one or more inserted amino acids that are not present in the native (unmodified) form of the clostridial neurotoxin. By way of example, a modified clostridial neurotoxin may have modified amino acid sequences in one or more domains relative to the native (unmodified) clostridial neurotoxin sequence. Such modifications may modify functional aspects of the toxin, for example biological activity or persistence. Thus, in one embodiment, the clostridial neurotoxin of the invention is a modified clostridial neurotoxin, or a modified clostridial neurotoxin derivative, or a clostridial neurotoxin derivative.

A modified clostridial neurotoxin may have one or more modifications in the amino acid sequence of the heavy chain (such as a modified H_{C} domain), wherein said modified heavy chain binds to target nerve cells with a higher or lower affinity than the native (unmodified) clostridial neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified clostridial neurotoxins are described in WO 2006/027207 and WO 2006/114308, both of which are hereby incorporated by reference in their entirety.

Thus, a BoNT/A H_{CC} domain is preferably a modified BoNT/A H_{CC} domain, more preferably a modified BoNT/A H_{C} domain. Therefore, preferably, a clostridial neurotoxin in accordance with the present invention is a modified BoNT/A. Preferably, said modified clostridial neurotoxin comprises one or more modifications that increases the isoelectric point of the clostridial neurotoxin when compared to an equivalent unmodified clostridial neurotoxin lacking said one or more modifications. Suitable modified clostridial neurotoxins are described below and in WO 2015/004461 A1 and WO 2016/110662 A1, which are incorporated herein by reference. Exemplary sequences include SEQ ID NOs: 14-17 (preferably SEQ ID NO: 14-mrBoNT/A) described herein.

A modified BoNT/A may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such a modified BoNT/A may demonstrate a reduction in, or absence of, side effects compared to the use of known BoNT/A. Said modified BoNT/A may exhibit increased tissue retention properties, thereby providing increased potency and/or duration of action and can allow for reduced dosages to be used compared to known clostridial toxin therapeutics (or increased dosages without any additional adverse effects), thus providing further advantages.

The modification may be a modification when compared to a BoNT/A shown as SEQ ID NO: 12, wherein the amino acid residue numbering is determined by alignment with SEQ ID NO: 12. As the presence of a methionine residue at position 1 of SEQ ID NO: 12 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 12 includes a methionine, the position numbering will be as defined above (e.g. ASN 886 will be ASN 886 of SEQ ID NO: 12). Alternatively, where the methionine is absent from SEQ ID NO: 12 the amino acid residue numbering should be modified by −1 (e.g. ASN 886 will be ASN 885 of SEQ ID NO: 12). Similar considerations apply when the methionine at position 1 of the other polypeptide sequences described herein is present/absent, and the skilled person will readily determine the correct amino acid residue numbering using techniques routine in the art.

An alignment described herein for determining amino acid residue numbering may be carried out using any of the methods described herein for determining sequence homology and/or % sequence identity.

The amino acid residue(s) indicated for modification above are surface exposed amino acid residue(s).

A modified BoNT/A may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of a modified BoNT/A preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A may comprise 1-30, 3-20, or 5-10 amino acid modifications. More preferably, the term “one or more amino acid residue(s)” when used in the context of a modified BoNT/A means all of the indicated amino acid residue(s).

Preferably, beyond the one or more amino acid modification(s) at the indicated amino acid residue(s), the modified BoNT/A does not contain any further amino acid modifications when compared to SEQ ID NO: 12.

The Modification May be Selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A that has an increased positive surface charge and increased isoelectric point when compared to the corresponding unmodified BoNT/A (e.g. SEQ ID NO: 12). Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action.

The isoelectric point (pl) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pl). The isoelectric point is a standard concept in protein biochemistry with which the skilled person would be familiar.

The isoelectric point (pl) is therefore defined as the pH value at which a protein displays a net charge of zero. An increase in pl means that a higher pH value is required for the protein to display a net charge of zero. Thus, an increase in pl represents an increase in the net positive charge of a protein at a given pH. Conversely, a decrease in pl means that a lower pH value is required for the protein to display a net charge of zero. Thus, a decrease in pl represents a decrease in the net positive charge of a protein at a given pH.

Methods of determining the pl of a protein are known in the art and would be familiar to a skilled person. By way of example, the pl of a protein can be calculated from the average pKa values of each amino acid present in the protein (“calculated pl”). Such calculations can be performed using computer programs known in the art, such as the Compute pl/MW Tool from ExPASy (https://web.expasy.org/compute_pi/), which is the preferred method for calculating pl in accordance with the present invention. Comparisons of pl values between different molecules should be made using the same calculation technique/program.

Where appropriate, the calculated pl of a protein can be confirmed experimentally using the technique of isoelectric focusing (“observed pl”). This technique uses electrophoresis to separate proteins according to their pl. Isoelectric focusing is typically performed using a gel that has an immobilised pH gradient. When an electric field is applied, the protein migrates through the pH gradient until it reaches the pH at which it has zero net charge, this point being the pl of the protein. Results provided by isoelectric focusing are typically relatively low-resolution in nature, and thus the present inventors believe that results provided by calculated pl (as described above) are more appropriate to use.

Throughout the present specification, “pl” means “calculated pl” unless otherwise stated.

The pl of a protein may be increased or decreased by altering the number of basic and/or acidic groups displayed on its surface. This can be achieved by modifying one or more amino acids of the protein. For example, an increase in pl may be provided by reducing the number of acidic residues, or by increasing the number of basic residues.

A modified BoNT/A of the invention may have a pl value that is at least 0.2, 0.4, 0.5 or 1 pl units higher than that of BoNT/A (e.g. SEQ ID NO: 12). Preferably, a modified BoNT/A may have a pl of at least 6.6, e.g. at least 6.8.

The properties of the 20 standard amino
acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A. In an amino acid substitution, an amino acid residue that forms part of the BoNT/A polypeptide sequence is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Following modification in accordance with the invention, the modified BoNT/A is preferably capable of binding to the target cell receptors that unmodified BoNT/A (e.g. SEQ ID NO: 12) binds.

Preferably, a modified BoNT/A for use in the invention comprises between 4 and 40 amino acid modifications located in the clostridial toxin H_{CN} domain. Said modified BoNT/A preferably also has pl of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

A modified BoNT/A may comprise a polypeptide sequence having at least 70% sequence identity to any one of SEQ ID NOs: 14-17. In one embodiment, a modified BoNT/A may comprise a polypeptide sequence having at least 80%, 85%, 90%, 95%, 99%, or 99.9% sequence identity to any one of SEQ ID NOs: 14-17. Preferably, a modified BoNT/A may comprise any one of SEQ ID NOs: 14-17. A modified BoNT/A may consist of a polypeptide sequence having at least 70% sequence identity to any one of SEQ ID NOs: 14-17. In one embodiment, a modified BoNT/A may consist of a polypeptide sequence having at least 80%, 85%, 90%, 95%, 99%, or 99.9% sequence identity to any one of SEQ ID NOs: 14-17. Preferably, a modified BoNT/A may consist of any one of SEQ ID NOs: 14-17. Of the recited SEQ ID NOS, SEQ ID NO: 14 is most preferred. The skilled person will appreciate that where the polypeptide sequence of a modified BoNT/A varies compared to a given SEQ ID NO by way of % sequence identity, that the at least one of the modifications (e.g. that increase pl) are still present (e.g. unmodified) in the variant modified BoNT/A.

Thus, a composition of the invention preferably comprises a modified BoNT/A, such as a modified BoNT/A as described above. In some embodiments, the clostridial neurotoxin polypeptides are modified BoNT/A polypeptides, the L-chain is a BoNT/A L-chain, the H_{N} domain is a BoNT/A H_{N} domain, and the H_{CC} domain (e.g. H_{C} domain) is a modified BoNT/A H_{CC} domain (e.g. a modified BoNT/A H_{CC} domain).

A modified BoNT/A may be encoded by a nucleotide sequence comprising at least 70% sequence identity to SEQ ID NO: 49. In one embodiment, a modified BoNT/A may be encoded by a nucleotide sequence comprising at least 80%, 85%, 90%, 95%, 99%, or 99.9% sequence identity to SEQ ID NO: 49. Preferably, a modified BoNT/A may be encoded by a nucleotide sequence comprising (more preferably consisting of) SEQ ID NO: 49. The skilled person will appreciate that where the nucleotide sequence encoding a modified BoNT/A varies compared to a given SEQ ID NO by way of % sequence identity, that the modified BoNT/A encoded still comprises at least one of the modifications (e.g. that increase pl), thus the relevant regions of the nucleotide sequence encoding said at least one of the modifications are still present (e.g. unmodified) in the variant nucleotide sequence.

A modified BoNT/A may comprise a substitution at one or more (preferably two or more, three or more, four or more, five or more or six or more, more preferably at all) of positions 930, 955, 991, 1026, 1052, 1229, and 886. Preferably, a modified BoNT/A comprises lysine or arginine (more preferably lysine) at one or more of positions 930, 955, 991, 1026, 1052, 1229, and 886. In one embodiment, the modified BoNT/A comprises lysine or arginine (more preferably lysine) at least two, three, four, five, six or all of positions 930, 955, 991, 1026, 1052, 1229, and 886. Most preferably, the modified BoNT/A comprises lysine or arginine (more preferably lysine) at all of positions 930, 955, 991, 1026, 1052, 1229, and 886.

A clostridial neurotoxin may comprise (or consist of) a hybrid or chimeric clostridial neurotoxin. A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may comprise the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety.

A clostridial neurotoxin of the invention may be one comprising a BoNT/B H_{CC} domain (e.g. a chimeric clostridial neurotoxin comprising a BoNT/B H_{CC} domain), preferably a chimeric clostridial neurotoxin comprising a BoNT/B H_{C} domain. Thus, in a particularly preferred embodiment, a clostridial neurotoxin of the invention may be a chimeric clostridial neurotoxin comprising (preferably consisting of) a BoNT/A light-chain and translocation domain (LH_{N} domain), and a BoNT/B receptor binding domain (H_{C} domain). Most preferably, said BoNT/B H_{C} domain comprises the following substitutions E1191M and S1199Y. A suitable chimeric clostridial neurotoxin may be one taught in WO 2017/191315 A1, which is incorporated herein by reference. Such preferred sequences include SEQ ID NOs: 7-11, with SEQ ID NO: 7 being most preferred.

The BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å(=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3 BTA (http://www.rcsb.org/pdb/explore/explore.do?structureId=3 BTA) and 1 EPW (http://www.rcsb.org/pdb/explore/explore.do?structureId=1 EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3 BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3 BTA.pdb structure;
4. The superposed pdb files were inspected to locate the 3₁₀ helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | N/A | 1-872 | 873-1296 | ⁸⁷²NIINTS⁸⁷⁷ |
| (SEQ ID | | | | |
| NO: 12) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (a.k.a. SEQ | | | | |
| ID NO: 13) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8th residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and,
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and,
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure. Without wishing to be bound by theory, it is hypothesized that by not disrupting the four central amino acid residues of the 3₁₀ helix in the BoNT/AB chimera ensures an optimal conformation for the chimeric neurotoxin.

The LH_{N} domain from BoNT/A may correspond to amino acid residues 1 to 872 of SEQ ID NO: 12, or a polypeptide sequence having at least 70% sequence identity thereto. The LH_{N} domain from BoNT/A may correspond to amino acid residues 1 to 872 of SEQ ID NO: 12, or a polypeptide sequence having at least 80%, 90% or 95% sequence identity thereto. Preferably, the LH_{N} domain from BoNT/A corresponds to amino acid residues 1 to 872 of SEQ ID NO: 12.

The H_{C} domain from BoNT/B may correspond to amino acid residues 860 to 1291 of SEQ ID NO: 13, or a polypeptide sequence having at least 70% sequence identity thereto. The H_{C} domain from BoNT/B may correspond to amino acid residues 860 to 1291 of SEQ ID NO: 13, or a polypeptide sequence having at least 80%, 90% or 95% sequence identity thereto. Preferably, the H_{C} domain from BoNT/B corresponds to amino acid residues 860 to 1291 of SEQ ID NO: 13.

Preferably, the LH_{N} domain corresponds to amino acid residues 1 to 872 of BoNT/A (SEQ ID NO: 12) and the H_{C} domain corresponds to amino acid residues 860 to 1291 of BoNT/B (SEQ ID NO: 13).

Preferably, a BoNT/B H_{C} domain further comprises at least one amino acid residue substitution, addition or deletion in the H_{CC} domain (e.g. subdomain) which has the effect of increasing the binding affinity of BoNT/B neurotoxin for human Syt II as compared to the natural BoNT/B sequence. Suitable amino acid residue substitution, addition or deletion in the BoNT/B H_{CC} domain have been disclosed in WO 2013/180799 and in WO 2016/154534 (both herein incorporated by reference).

Suitable amino acid residue substitution, addition or deletion in the BoNT/B H_{CC} domain include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} domain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} domain also include a combination of three substitution mutations which are E1191M, S1199W and W1178Q.

Preferably, the suitable amino acid residue substitution, addition or deletion in the BoNT/B H_{CC} domain includes a combination of two substitution mutations which are E1191M and S1199Y.

The modification may be a modification when compared to unmodified BoNT/B shown as SEQ ID NO: 13, wherein the amino acid residue numbering is determined by alignment with SEQ ID NO: 13. As the presence of a methionine residue at position 1 of SEQ ID NO: 13 is optional, the skilled person will take the presence/absence of the methionine residue into account when determining amino acid residue numbering. For example, where SEQ ID NO: 13 includes a methionine, the position numbering will be as defined above (e.g. E1191 will be E1191 of SEQ ID NO: 13). Alternatively, where the methionine is absent from SEQ ID NO: 13 the amino acid residue numbering should be modified by −1 (e.g. E1191 will be E1190 of SEQ ID NO: 13). Similar considerations apply when the methionine at position 1 of the other polypeptide sequences described herein is present/absent, and the skilled person will readily determine the correct amino acid residue numbering using techniques routine in the art.

A chimeric clostridial neurotoxin may comprise a polypeptide sequence having at least 70% sequence identity to any one of SEQ ID NOs: 7-11. In one embodiment, a chimeric clostridial neurotoxin may comprise a polypeptide sequence having at least 80%, 85%, 90%, 95%, 99%, or 99.9% sequence identity to any one of SEQ ID NOs: 7-11. Preferably, a chimeric clostridial neurotoxin may comprise any one of SEQ ID NOs: 7-11. A chimeric clostridial neurotoxin may consist of a polypeptide sequence having at least 70% sequence identity to any one of SEQ ID NOs: 7-11. In one embodiment, a chimeric clostridial neurotoxin may consist of a polypeptide sequence having at least 80%, 85%, 90%, 95%, 99%, or 99.9% sequence identity to any one of SEQ ID NOs: 7-11. Preferably, a chimeric clostridial neurotoxin may consist of any one of SEQ ID NOs: 7-11. Of the recited SEQ ID NOS, SEQ ID NO: 7 is most preferred. The skilled person will appreciate that where the polypeptide sequence of a chimeric clostridial neurotoxin comprising at least one BoNT/B H_{CC} domain mutation varies compared to a given SEQ ID NO by way of % sequence identity, that the at least one BoNT/B H_{CC} domain mutations (preferably E1191M and S1199Y) are present (e.g. are unmodified) in the variant chimeric clostridial neurotoxin.

Thus, a composition of the invention most preferably comprises a chimeric clostridial neurotoxin, such as a chimeric clostridial neurotoxin as described above. In some embodiments, the clostridial neurotoxin polypeptides are chimeric clostridial neurotoxin polypeptides, the L-chain is a BoNT/A L-chain, the H_{N} domain is a BoNT/A H_{N} domain, and the H_{CC} domain (e.g. H_{C} domain) is a BoNT/B H_{CC} domain (e.g. a BoNT/B H_{CC} domain).

In another embodiment, a clostridial neurotoxin of the invention may be a chimeric clostridial neurotoxin comprising a BoNT/X light-chain and translocation domain (LH_{N} domain), and a receptor binding domain (H_{C} domain) or a portion thereof from a different (i.e. non-BoNT/X) clostridial neurotoxin. A suitable chimeric and/or hybrid clostridial neurotoxin may be one taught in WO 2020/065336 A1, which is incorporated herein by reference.

In embodiments where a clostridial neurotoxin described herein has a tag for purification (e.g. a His-tag) and/or a linker, said tag and/or linker are optional.

The clostridial neurotoxins of the present invention may be free from the complexing proteins that are present in a naturally occurring clostridial neurotoxin complex.

The clostridial neurotoxins of the present invention can be produced using recombinant nucleic acid technologies. Thus, in one embodiment, a clostridial neurotoxin (as described above) is a recombinant clostridial neurotoxin.

In one embodiment a nucleic acid (for example, a DNA) comprising a nucleic acid sequence encoding a clostridial neurotoxin is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |

In another preferred embodiment, the vector has a promoter selected from:

| Promoter | Induction Agent | Typical Induction Condition |
|---|---|---|
| Tac (hybrid) | IPTG | 0.2 mM (0.05-2.0 mM) |
| AraBAD | L-arabinose | 0.2% (0.002-0.4%) |
| T7-lac operator | IPTG | 0.2 mM (0.05-2.0 mM) |
| T5-lac operator | IPTG | 0.2 mM (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A clostridial neurotoxin of the invention is preferably present as a di-chain clostridial neurotoxin in which the L-chain is linked to the H-chain (or component thereof, e.g. the H_{N} domain) via a di-sulphide bond. Thus, a clostridial neurotoxin of the invention may be any clostridial neurotoxin or variant (expressed by way of % sequence identity to a given SEQ ID NO) herein that has been cleaved by a protease in its activation loop (at one or more sites).

A clostridial neurotoxin preferably comprises an L-chain and H-chain, wherein the L-chain and H-chain are joined by a di-sulphide bond and are obtainable by (e.g. obtained by) cleaving a polypeptide comprising at least 70% sequence identity to SEQ ID NO: 14 with a protease in its activation loop at one or more sites. In one embodiment, a clostridial neurotoxin comprises an L-chain and H-chain, wherein the L-chain and H-chain are joined by a di-sulphide bond and are obtainable by (e.g. obtained by) cleaving a polypeptide comprising at least 80%, 85%, 90%, 95%, 99%, or 99.9% sequence identity to SEQ ID NO: 14 with a protease in its activation loop at one or more sites. Preferably, a clostridial neurotoxin comprises an L-chain and H-chain, wherein the L-chain and H-chain are joined by a di-sulphide bond and are obtainable by (e.g. obtained by) cleaving a polypeptide comprising SEQ ID NO: 14 with a protease in its activation loop at one or more sites.

A clostridial neurotoxin most preferably comprises an L-chain and H-chain, wherein the L-chain and H-chain are joined by a di-sulphide bond and are obtainable by (e.g. obtained by) cleaving a polypeptide comprising at least 70% sequence identity to SEQ ID NO: 7 with a protease in its activation loop at one or more sites. In one embodiment, a clostridial neurotoxin comprises an L-chain and H-chain, wherein the L-chain and H-chain are joined by a di-sulphide bond and are obtainable by (e.g. obtained by) cleaving a polypeptide comprising at least 80%, 85%, 90%, 95%, 99%, or 99.9% sequence identity to SEQ ID NO: 7 with a protease in its activation loop at one or more sites. Preferably, a clostridial neurotoxin comprises an L-chain and H-chain, wherein the L-chain and H-chain are joined by a di-sulphide bond and are obtainable by (e.g. obtained by) cleaving a polypeptide comprising SEQ ID NO: 7 with a protease in its activation loop at one or more sites.

In a particularly preferred embodiment, a di-chain clostridial neurotoxin comprises (or consists of) a light-chain comprising a polypeptide sequence having at least 70%, 80%, 90%, 95%, or 99.9% sequence identity to SEQ ID NO: 77 or 78 (preferably SEQ ID NO: 77) and a heavy-chain comprising a polypeptide sequence having at least 70%, 80%, 90%, 95%, or 99.9% sequence identity to SEQ ID NO: 79, wherein the light-chain and heavy-chain are joined together by a di-sulphide bond. More preferably, a di-chain clostridial neurotoxin comprises (or consists of) a light-chain comprising SEQ ID NO: 77 or 78 (preferably SEQ ID NO: 77) and a heavy-chain comprising SEQ ID NO: 79, wherein the light-chain and heavy-chain are joined together by a di-sulphide bond. Even more preferably, a di-chain clostridial neurotoxin comprises (or consists of) a light-chain having SEQ ID NO: 77 and a heavy-chain having SEQ ID NO: 79, wherein the light-chain and heavy-chain are joined together by a di-sulphide bond. The di-sulphide bond is preferably formed by and/or is between the cysteine residue at position 429 of SEQ ID NO: 77 or 78 and the cysteine residue at position 6 of SEQ ID NO: 79. Said di-chain clostridial neurotoxin may correspond to a di-chain form of SEQ ID NO: 7.

The protease used to cleave the activation loop is preferably Lys-C. Suitable proteases and method for cleaving activation loops to produce di-chain clostridial neurotoxins are taught in WO 2014/080206, WO2014/079495, and EP2677029A2, which are incorporated herein by reference.

Suitable activation loop sequences are shown in the table below:

| | | | | SEQ |
|---|---|---|---|---|
| | aa | | aa | ID |
| Protein id | start | Sequence | end | NO: |
| BoNT/D_AB012112 | 437 | CLRLTKNSRDDSTC | 450 | 52 |
| BoNT/DC_AB745660 | 437 | CLRLTRNSRDDSTC | 450 | 53 |
| BoNT/C1_X62389 | 437 | CHKAIDGRSLYNKTLDC | 453 | 54 |
| BoNT/CD_AB200360 | 437 | CHKAIDGRSLYNKTLDC | 453 | |
| BoNT/A4_EU341307 | 430 | CVRGIITSKTKSLDEGYNKALNELC | 454 | 55 |
| BoNT/A7_JQ954969 | 430 | CVRGIITSKTKSLDEGYNKALNDLC | 454 | 56 |
| BoNT/A6_FJ981696 | 430 | CVRGIITSKTKSLDKGYNKALNDLC | 454 | 57 |
| BoNT/A1_AF488749 | 430 | CVRGIITSKTKSLDKGYNKALNDLC | 454 | |
| BoNT/A5_EU679004 | 430 | CVRGIITSKTKSLDEGYNKALNDLC | 454 | 56 |
| BoNT/A3_DQ185900 | 426 | CVRGIIPFKTKSLDEGYNKALNYLC | 450 | 58 |
| BoNT/A2_X73423 | 430 | CVRGIIPFKTKSLDEGYNKALNDLC | 454 | 59 |
| BoNT/A8_KM233166 | 430 | CVRGIIPFKTKSLDEGYNKALNDLC | 454 | |
| BoNT/H_KGO15617 | 428 | CSNSNTKNSLC | 438 | 60 |
| BoNT/E9_JX424534 | 414 | CKNIVSVKGIRKSIC | 429 | 61 |
| BoNT/E12_KM370319 | 414 | CKNIVSVKGIRKSIC | 429 | |
| BoNT/E11_KF861875 | 411 | CTNIFSPKGIRKSIC | 426 | 62 |
| BoNT/E10_KF861917 | 411 | CKNIVFSKGITKSIC | 426 | 63 |
| BoNT/E7_JN695729 | 411 | CKNIVFSKGITKSIC | 426 | |
| BoNT/E8_JN695730 | 411 | CKNIVFSKGITKSIC | 426 | |
| BoNT/E5_AB037711 | 411 | CKNIVSVKGIRKSIC | 426 | 61 |
| BoNT/E6_AM695759 | 411 | CKNIVFSKGIRKSIC | 426 | 64 |
| BoNT/E4_AB088207 | 411 | CKNIVSVKGIRKSIC | 426 | 61 |
| BoNT/E3_EF028403 | 411 | CKNIVSVKGIRKSIC | 426 | |
| BoNT/E1_GQ244314 | 411 | CKNIVSVKGIRKSIC | 426 | |
| BoNT/E2_EF028404 | 411 | CKNIVSVKGIRKSIC | 426 | |
| BoNT/F7_GU213233 | 420 | CKSIVSKKGTKNSLC | 434 | 65 |
| BoNT/F5_GU213211 | 428 | CLNSSFKKNTKKPLC | 442 | 66 |
| BoNT/F1_GU213203 | 429 | CKSVIPRKGTKAPPRLC | 445 | 67 |
| BoNT/F4_GU213214 | 429 | CKSIIPRKGTKAPPRLC | 445 | 68 |
| BoNT/F2_GU213209 | 429 | CKSIIPRKGTKQSPSLC | 445 | 69 |
| BoNT/F3_GU213227 | 429 | CKSIIPRKGTKQSPSLC | 445 | |
| BoNT/F6_M92906 | 429 | CKSVIPRKGTKAPPRLC | 445 | 67 |
| TeNT_P04958 | 439 | CKKIIPPTNIRENLYNRTASLTDLGGEL | 467 | 70 |
| | | C | | |
| BoNT/G_X74162 | 436 | CKPVMYKNTGKSEQC | 450 | 71 |
| BoNT/B4_EF051570 | 437 | CKSVKVPGIC | 446 | 72 |
| BoNT/B8_JQ964806 | 437 | CKSVRAPGIC | 446 | 73 |
| BoNT/B7_JQ354985 | 437 | CKSVKAPGIC | 446 | 74 |
| BoNT/B6_AB302852 | 437 | CKSVRAPGIC | 446 | 73 |
| BoNT/B2_AB084152 | 437 | CKSVRAPGIC | 446 | |
| BoNT/B3_EF028400 | 437 | CKSVRAPGIC | 446 | |

Lys-C may cleave an activation loop C-terminal to one or more of the lysine residues present therein. Where Lys-C cleaves the activation loop more than once, the skilled person will appreciate that a small peptide of the activation loop of a di-chain clostridial neurotoxin may be absent when compared to a SEQ ID NO shown herein. For example, SEQ ID NO: 75 or 76 may be absent.

The invention provides a method of producing a single-chain clostridial neurotoxin having a light chain and a heavy chain, the method comprising expressing a nucleic acid described herein in an expression host, lysing the host cell to provide a host cell homogenate containing the single-chain clostridial neurotoxin, and isolating the single-chain clostridial neurotoxin. In one aspect, the present invention provides a method of proteolytically processing a clostridial neurotoxin described herein, the method comprising contacting the clostridial neurotoxin with a protease that hydrolyses a peptide bond in the activation loop of the clostridial neurotoxin, thereby converting the (single-chain) clostridial neurotoxin into a corresponding di-chain clostridial neurotoxin (e.g., wherein the light chain and heavy chain are joined together by a disulphide bond).

The present invention therefore provides a di-chain clostridial neurotoxin obtainable by a method of the invention.

Where an initial methionine amino acid residue or a corresponding initial codon is indicated in any of the SEQ ID NOs disclosed herein, said residue/codon is optional. Preferably, said initial methionine amino acid residue or corresponding initial codon is absent.

A composition for use in a method of the invention may be a first clostridial neurotoxin formulation comprising one or more pharmaceutically acceptable carrier(s), excipient(s), adjuvant(s), propellant(s), and/or salt(s).

A method may further comprise determining the clostridial neurotoxin activity of at least a second clostridial neurotoxin formulation, wherein the at least second clostridial neurotoxin formulation comprises the same clostridial neurotoxin present in the same amount as the first clostridial neurotoxin formulation and one or more pharmaceutically acceptable carrier(s), excipient(s), adjuvant(s), propellant(s), and/or salt(s), wherein the one or more pharmaceutically acceptable carrier(s), excipient(s), adjuvant(s), propellant(s), and/or salt(s) is different to the one or more pharmaceutically acceptable carrier(s), excipient(s), adjuvant(s), propellant(s), and/or salt(s) present in the first clostridial neurotoxin formulation.

A method may comprise comparing the clostridial neurotoxin activity of the first clostridial neurotoxin formulation and the at least second clostridial neurotoxin formulation, and selecting the one or more pharmaceutically acceptable carrier(s), excipient(s), adjuvant(s), propellant(s), and/or salt(s) when the clostridial neurotoxin formulation comprising the same exhibits the highest activity.

The methods as described above may allow for selection of preferred pharmaceutically acceptable carrier(s), excipient(s), adjuvant(s), propellant(s), and/or salt(s) for formulating a clostridial neurotoxin.

A method may further comprise determining the clostridial neurotoxin activity of at least a second clostridial neurotoxin formulation, wherein the at least second clostridial neurotoxin formulation comprises the same clostridial neurotoxin present in the same amount as the first clostridial neurotoxin formulation and one or more pharmaceutically acceptable carrier(s), excipient(s), adjuvant(s), propellant(s), and/or salt(s), wherein the one or more pharmaceutically acceptable carrier(s), excipient(s), adjuvant(s), propellant(s), and/or salt(s) is the same as the one or more pharmaceutically acceptable carrier(s), excipient(s), adjuvant(s), propellant(s), and/or salt(s) present in the first clostridial neurotoxin formulation but present in a different amount (e.g. a different concentration).

A method may comprise comparing the clostridial neurotoxin activity of the first clostridial neurotoxin formulation and the at least second clostridial neurotoxin formulation, and selecting the amount of the one or more pharmaceutically acceptable carrier(s), excipient(s), adjuvant(s), propellant(s), and/or salt(s) when the clostridial neurotoxin formulation comprising the same exhibits the highest activity.

The methods as described above may allow for selection of preferred amounts of pharmaceutically acceptable carrier(s), excipient(s), adjuvant(s), propellant(s), and/or salt(s) for formulating a clostridial neurotoxin.

In one aspect, the invention provides a method for producing a therapeutic or cosmetic clostridial neurotoxin composition, the method comprising:
(a) obtaining the results of the method according to the invention, and formulating and/or packaging the composition for therapeutic or cosmetic use when the clostridial neurotoxin activity (e.g. an activity level) is the same as or higher than a positive control; or
(b) subjecting the composition to further purification when the clostridial neurotoxin activity is lower than a positive control, and formulating and/or packaging the further purified composition for therapeutic or cosmetic use.

In one aspect, there is provided a therapeutic or cosmetic clostridial neurotoxin composition obtainable by a method of the invention, optionally wherein the therapeutic or cosmetic clostridial neurotoxin composition is packaged.

The term “obtainable” as used herein also encompasses the term “obtained”.

In one aspect, the invention provides an isolated capture substrate for a clostridial neurotoxin, wherein the capture substrate comprises an extracellular portion of a clostridial neurotoxin receptor polypeptide that comprises an amino acid modification and/or a post-translational modification. The isolated capture substrate may be complexed with a clostridial neurotoxin, wherein an H_{CC} domain (e.g. H_{C} domain) of the clostridial neurotoxin is bound to the extracellular portion of the clostridial neurotoxin receptor polypeptide. The isolated capture substrate may be complexed with a clostridial neurotoxin receptor binding domain (H_{CC} domain or H_{C} domain). Preferably, an isolated capture substrate comprises an extracellular portion of SYT-II comprising a L51F substitution as described herein. Alternatively, an isolated capture substrate may comprise an extracellular portion of SV2c comprising a post-translational modification (preferably glycosylation) as described herein. An isolated capture substrate may refer to a capture substrate that has been isolated from a cell. Such a capture substrate may have been produced recombinantly and isolated using standard techniques. Thus, in some embodiments, the term “isolated capture substrate” is intended to encompass capture substrates in an in vitro environment. Preferably, an isolated capture substrate is immobilised on a solid support as described herein for capture substrates generally.

In one aspect, the invention provides the use of an isolated capture substrate for a clostridial neurotoxin for determining the presence or absence of an activity-altering property of clostridial neurotoxin polypeptides comprised in a composition, wherein the isolated capture substrate comprises an extracellular portion of a clostridial neurotoxin receptor polypeptide that comprises an amino acid modification and/or a post-translational modification.

An activity-altering property may be an activity-increasing or activity-decreasing property, preferably an activity-decreasing property. In one embodiment, the activity-altering property may be any property that alters activity of a clostridial neurotoxin polypeptide when compared to an otherwise identical clostridial neurotoxin (preferably an otherwise identical active di-chain clostridial neurotoxin) lacking said property. In one embodiment, the activity-altering property may be any property that alters activity of a portion of a clostridial neurotoxin (e.g. the L-chain or H-chain or portions thereof, such as the H_{C} or H_{CC} domain) when compared to an otherwise identical portion of a clostridial neurotoxin lacking said property. Thus, the activity-altering property may be a topological change and/or a structural change. The activity-altering property may be a modification (e.g. a post-translational modification), such as a covalent modification and/or a non-covalent modification. In one embodiment, an activity-altering property is deamidation, fragmentation (e.g. truncation of a clostridial neurotoxin polypeptide, e.g. resulting in the loss of a domain), aggregation, oxidation (e.g. of a methionine residue or tryptophan), glycation (e.g. of a lysine residue), lactosylation, incorrect di-sulphide bond formation (e.g. resulting in loss of the L-chain or loss of the H-chain), incorrect charge (e.g. an incorrect charge profile), and/or an intact activation loop (e.g. a composition may be contaminated with single-chain clostridial neurotoxin polypeptides). Such activity-altering properties are preferably activity-decreasing properties. Preferably, an activity-decreasing property is oxidation.

An example of an activity-altering property of an L-chain may include an intact activation loop, resulting in an L-chain polypeptide that is inactive. This may arise in instances where proteolytic cleavage of a single-chain clostridial neurotoxin into the active di-chain form has failed and/or is inefficient. Other activity-altering properties of an L-chain may include a topological change, a structural change, deamidation, fragmentation, aggregation, oxidation (e.g. of a methionine residue or tryptophan), glycation (e.g. of a lysine residue), lactosylation, incorrect di-sulphide bond formation, and/or incorrect charge (e.g. an incorrect charge profile).

An example of an activity-altering property of an H-chain (e.g. H_{C} domain) may include a topological change, a structural change, deamidation, fragmentation, aggregation, oxidation (e.g. of a methionine residue or tryptophan), glycation (e.g. of a lysine residue), lactosylation, and/or incorrect charge (e.g. an incorrect charge profile).

Amino acids that may be oxidised comprise methionine, cysteine, histidine, tryptophan, tyrosine, and/or phenylalanine (Torosantucci et al (2014), Pharm Res, 31, 541-553).

An activity-altering property may be one that changes the activity of a clostridial neurotoxin (or portion thereof, e.g. the L-chain or H-chain or portions thereof, such as the H_{C} or H_{CC} domain) by at least 1%, 2%, 5%, 10%, 25%, 50% or 100% when compared to an otherwise identical clostridial neurotoxin (or portion thereof, e.g. the L-chain or H-chain or portions thereof, such as the H_{C} or H_{CC} domain) lacking said property. An activity-increasing property may be one that increases the activity of a clostridial neurotoxin polypeptide (or portion thereof, e.g. the L-chain or H-chain or portions thereof, such as the H_{C} or H_{CC} domain) by at least 1%, 2%, 5%, 10%, 25%, 50% or 100% when compared to an otherwise identical clostridial neurotoxin (or portion thereof, e.g. the L-chain or H-chain or portions thereof, such as the H_{C} or H_{CC} domain) lacking said property. An activity-decreasing property may be one that decreases the activity of a clostridial neurotoxin polypeptide (or portion thereof, e.g. the L-chain or H-chain or portions thereof, such as the H_{C} or H_{CC} domain) by at least 1%, 2%, 5%, 10%, 25%, 50% or 100% when compared to an otherwise identical clostridial neurotoxin (or portion thereof, e.g. the L-chain or H-chain or portions thereof, such as the H_{C} or H_{CC} domain) lacking said property.

An activity-altering property is preferably an activity-altering property of the H-chain (e.g. an activity-altering modification of the H-chain), more preferably an activity-decreasing property of the H_{C} domain, such as an activity-decreasing modification present in the H_{C} domain.

The inventors have found that by combining a method of the invention with one or more additional methods, valuable insights into the nature of a clostridial neurotoxin composition may be obtained (see Example 3, for example). This may allow for the interrogation of quality and/or efficacy of such a composition in a domain-specific manner. Advantageously, this is much improved when compared to a cell-based assay which is not able to provide a mechanistic insight into the specific cause of a change in potency.

Thus, in one embodiment, a method of the invention may further comprise obtaining the results of a cell-free substrate cleavage assay. In one embodiment, a method of the invention may further comprise obtaining the results of a heavy-chain binding assay. Preferably, a method of the invention may further comprise obtaining the results of a cell-free substrate cleavage assay and the results of a heavy-chain binding assay.

In one embodiment, said results may be obtained prior to carrying out a method of the invention. In some embodiments, a negative result or a result that is lower than a control as determined by a cell-free substrate cleavage assay and/or heavy-chain binding assay may necessitate carrying out a method of the invention.

The results of a cell-free substrate cleavage assay (e.g. endopeptidase assay) and/or the results of a heavy-chain binding assay may be compared with the results of the present invention (e.g. the determined clostridial neurotoxin activity of the composition). Said comparison may allow for the determination as to whether clostridial neurotoxin polypeptides (or portion thereof, e.g. the L-chain or H-chain or portions thereof, such as the H_{C} or H_{CC} domain) comprised in a composition comprise an activity-altering property. Said comparison may allow for the determination as to an amount of clostridial neurotoxin polypeptides (or portion thereof, e.g. the L-chain or H-chain or portions thereof, such as the H_{C} or H_{CC} domain) comprised in a composition that comprise an activity-altering property. Preferably, the results of a cell-free substrate cleavage assay and the results of a heavy-chain binding assay may be compared with the results of the present invention (e.g. the determined clostridial neurotoxin activity of the composition).

The heavy-chain binding control and/or (preferably and) substrate cleavage assay control may be negative controls. The heavy-chain binding control and/or (preferably and) substrate cleavage assay control are preferably positive controls. Suitable positive controls are described herein. It is preferred that the heavy-chain binding positive control is representative of a composition comprising clostridial neurotoxin polypeptides that do not comprise an activity-altering property of a clostridial neurotoxin H-chain, preferably an activity-altering property of the H_{C} domain. It is preferred that the substrate cleavage positive control is representative of a composition comprising clostridial neurotoxin polypeptides that do not comprise an activity-altering property of the L-chain.

Where the results of a heavy-chain binding assay are representative of lower heavy-chain binding of the composition when compared to a heavy-chain binding positive control, the results of a cell-free substrate cleavage assay are representative of the same L-chain activity of the composition when compared to a substrate cleavage positive control, and the results of a method of the invention are representative of a lower clostridial neurotoxin activity of the composition when compared to a positive control (e.g. positive reference standard), it may be determined that the clostridial neurotoxin polypeptides comprised in the composition comprise an activity-decreasing property of the H-chain, preferably the H_{C} domain.

Where the results of a heavy-chain binding assay are representative of higher heavy-chain binding of the composition when compared to a heavy-chain binding positive control, the results of a cell-free substrate cleavage assay are representative of the same L-chain activity of the composition when compared to a substrate cleavage positive control, and the results of a method of the invention are representative of a higher clostridial neurotoxin activity of the composition when compared to a positive control (e.g. positive reference standard), it may be determined that the clostridial neurotoxin polypeptides comprised in the composition comprise an activity-increasing property of the H-chain, preferably the H_{C} domain.

Where the results of a heavy-chain binding assay are representative of the same heavy-chain binding of the composition when compared to a heavy-chain binding positive control, the results of a cell-free substrate cleavage assay are representative of a lower L-chain activity of the composition when compared to a substrate cleavage positive control, and the results of a method of the invention are representative of a lower clostridial neurotoxin activity of the composition when compared to a positive control (e.g. positive reference standard), it may be determined that the clostridial neurotoxin polypeptides comprised in the composition comprise an activity-decreasing property of the L-chain.

Where the results of a heavy-chain binding assay are representative of the same heavy-chain binding of the composition when compared to a heavy-chain binding positive control, the results of a cell-free substrate cleavage assay are representative of a higher L-chain activity of the composition when compared to a substrate cleavage positive control, and the results of a method of the invention are representative of a higher clostridial neurotoxin activity of the composition when compared to a positive control (e.g. positive reference standard), it may be determined that the clostridial neurotoxin polypeptides comprised in the composition comprise an activity-increasing property of the L-chain.

The skilled person will appreciate that any difference may be quantified to determine an amount of polypeptides of the composition comprising a given activity-altering property.

### A Heavy-Chain Binding Assay May Comprise:

(a) providing capture substrates for the clostridial neurotoxin polypeptides;
(b) contacting the capture substrates with a composition comprising the clostridial neurotoxin polypeptides, for binding of the clostridial neurotoxin polypeptides to the capture substrates;
(c) removing unbound clostridial neurotoxin polypeptides; and
(d) determining an amount of the clostridial neurotoxin polypeptides bound to the capture substrates.

Thus, in one aspect, the invention provides a (cell-free) method assaying heavy-chain binding of clostridial neurotoxin polypeptides, the method comprising:
(a) providing capture substrates for the clostridial neurotoxin polypeptides;
(b) contacting the capture substrates with a composition comprising the clostridial neurotoxin polypeptides or a portion thereof comprising at least an H_{CC} or H_{C} domain of the clostridial neurotoxin polypeptide, for binding of the clostridial neurotoxin polypeptides or the portion thereof to the capture substrates;
(c) removing unbound clostridial neurotoxin polypeptides or the portion thereof; and
(d) determining an amount of the clostridial neurotoxin polypeptides or the portion thereof bound to the capture substrates.

The amount of clostridial neurotoxin polypeptides (or the portion thereof) bound to the capture substrates may be determined using any suitable method. For example, an antibody (suitably a polyclonal antibody) may be used that specifically binds to the clostridial neurotoxin polypeptides (or the portion thereof). The antibody may be detected using a secondary antibody that specifically binds to the antibody (suitably polyclonal antibody) that specifically binds to the clostridial neurotoxin polypeptides (or the portion thereof). Said secondary antibody may comprise a suitable detection means, such as a conjugated peroxidase enzyme (e.g. horseradish peroxidase), which can be detected by incubation with a chromogenic (e.g. DAB, TMB, OPD), fluorogenic (e.g. ADHP) or chemiluminescent (e.g. ECL) substrate. Chromogenic, fluorogenic and/or chemiluminescent changes (indicative of catalysis by peroxidase) may be used to quantify an amount of the clostridial neurotoxin polypeptides (or the portion thereof) bound to the capture substrates. Preferably, the substrate is TMB (3,3′,5,5′-Tetramethylbenzidine).

A heavy-chain binding assay may be an ELISA, such as one known in the art. Suitable methodology is provided in Example 3 herein. Another heavy-chain binding assay technique may be or may comprise bi-layer interferometry. Such a technique may employ the Octet Red 96e system.

The term “specifically” as used in the context of an antibody that specifically binds to an antigen (e.g. clostridial neurotoxin polypeptide) may mean that the antibody binds to the antigen with greater specificity and affinity than it binds to a non-antigen. In some embodiments said binding may be at least 10 times, 50 times, 100 times, 500 times, or 1000 times greater than the binding to the non-antigen.

A cell-free substrate cleavage assay preferably comprises the use of cleavable substrates described herein. In one embodiment, a cell-free substrate cleavage assay may be carried out in the same manner as a method of the invention but omitting capture substrates, contacting them with the composition, and removal of unbound clostridial neurotoxin polypeptides. A cell-free substrate cleavage assay may comprise:
(a) contacting a composition comprising clostridial neurotoxin polypeptides and a reducing agent with cleavable substrates; and
(b) determining an amount of cleavable substrates cleaved by the L-chain polypeptides of the clostridial neurotoxin polypeptides, thereby determining the L-chain activity of the composition.

Thus, in one aspect, the invention provides a cell-free method for assaying substrate cleavage comprising:
(a) contacting a composition comprising clostridial neurotoxin polypeptides, and a reducing agent with cleavable substrates; or
(b) contacting a composition comprising at least an L-chain of the clostridial neurotoxin polypeptides with cleavable substrates; and
(b) determining an amount of cleavable substrates cleaved by the L-chain polypeptides (e.g. of the clostridial neurotoxin polypeptides), thereby determining the L-chain activity of the composition.

Owing to the presence of a reducing agent the composition may comprise L-chain polypeptides that have been dissociated from the corresponding H-chain polypeptides of the clostridial neurotoxin polypeptides. Determining an amount of cleavable substrates cleaved by the L-chain polypeptides of the clostridial neurotoxin polypeptides may suitably be carried out using any method described herein. Preferably, the same cleavable substrates are employed and the methodology used is the same as that for a method of the invention.

Suitable cell-free substrate cleavage assay methodology is provided in Example 3 herein.

In one aspect, the invention provides the use of an isolated capture substrate for a clostridial neurotoxin for determining the presence or absence of activity-decreasing heavy-chain (H-chain, e.g. H_{C} or H_{CC} domain) oxidation of clostridial neurotoxin polypeptides comprised in a composition, wherein the isolated capture substrate comprises an extracellular portion of a clostridial neurotoxin receptor polypeptide that comprises an amino acid modification and/or a post-translational modification.

In one aspect, the invention provides a cell-free method for determining whether or not clostridial neurotoxin polypeptides or a portion thereof (e.g. an H-chain, such as an H_{C} or H_{CC} domain thereof) comprised in a composition comprise an activity-altering property, the method comprising:
(a) providing capture substrates for the clostridial neurotoxin polypeptides or the portion thereof (e.g. an H-chain, such as an H_{C} or H_{CC} domain thereof);
(b) contacting the capture substrates with the composition, for binding of the clostridial neurotoxin polypeptides or the portion thereof (e.g. an H-chain, such as an H_{C} or H_{CC} domain thereof) to the capture substrates;
(c) removing unbound clostridial neurotoxin polypeptides or the portion thereof (e.g. an H-chain, such as an H_{C} or H_{CC} domain thereof);
(d) determining an amount of the clostridial neurotoxin polypeptides or the portion thereof (e.g. an H-chain, such as an H_{C} or H_{CC} domain thereof) bound to the capture substrates;
(e) comparing the amount of bound clostridial neurotoxin polypeptides or the portion thereof (e.g. an H-chain, such as an H_{C} or H_{CC} domain thereof) with a control; and
(f) determining whether or not clostridial neurotoxin polypeptides or the portion thereof (e.g. an H-chain, such as an H_{C} or H_{CC} domain thereof) comprised in the composition comprise the activity-altering property (e.g. the activity-altering property of the H-chain) based on the comparison.

Preferably, the invention provides a cell-free method for determining whether or not clostridial neurotoxin polypeptides comprised in a composition comprise an activity-altering property, the method comprising:
(a) providing capture substrates for the clostridial neurotoxin polypeptides;
(b) contacting the capture substrates with the composition, for binding of the clostridial neurotoxin polypeptides to the capture substrates;
(c) removing unbound clostridial neurotoxin polypeptides;
(d) determining an amount of the clostridial neurotoxin polypeptides bound to the capture substrates;
(e) comparing the amount of bound clostridial neurotoxin polypeptides with a control; and
(f) determining whether or not clostridial neurotoxin polypeptides comprised in the composition comprise the activity-altering property (e.g. the activity-altering property of the H-chain) based on the comparison.

A suitable control may be any control described herein. A negative control may be representative of a composition comprising clostridial neurotoxin polypeptides or a portion thereof (e.g. an H-chain, such as an H_{C} or H_{CC} domain thereof) that comprise an activity-decreasing property. In such embodiments, when an amount of clostridial neurotoxin polypeptides or a portion thereof (e.g. an H-chain, such as an H_{C} or H_{CC} domain thereof) bound to the capture substrates is higher than said negative control, it may be determined that the clostridial neurotoxin polypeptides or a portion thereof (e.g. an H-chain, such as an H_{C} or H_{CC} domain thereof) comprised in the composition do not comprise an activity-decreasing property of the H-chain. In such embodiments, when an amount of clostridial neurotoxin polypeptides or a portion thereof (e.g. an H-chain, such as an H_{C} or H_{CC} domain thereof) bound to the capture substrates is the same as or lower than said negative control, it may be determined that the clostridial neurotoxin polypeptides or the portion thereof (e.g. an H-chain, such as an H_{C} or H_{CC} domain thereof) comprised in the composition comprise an activity-decreasing property of the H-chain.

The control is preferably a positive control, more preferably a heavy-chain binding positive control as described herein.

In one embodiment, when an amount of clostridial neurotoxin polypeptides or a portion thereof (e.g. an H-chain, such as an H_{C} or H_{CC} domain thereof) bound to the capture substrates is higher than the heavy-chain binding positive control, it may be determined that the clostridial neurotoxin polypeptides or the portion thereof (e.g. an H-chain, such as an H_{C} or H_{CC} domain thereof) comprised in the composition comprise an activity-increasing property of the H-chain.

In one embodiment, when an amount of clostridial neurotoxin polypeptides or a portion thereof (e.g. an H-chain, such as an H_{C} or H_{CC} domain thereof) bound to the capture substrates is lower than the heavy-chain binding positive control, it may be determined that the clostridial neurotoxin polypeptides or the portion thereof (e.g. an H-chain, such as an H_{C} or H_{CC} domain thereof) comprised in the composition comprise an activity-decreasing property of the H-chain.

In one embodiment, when an amount of clostridial neurotoxin polypeptides or a portion thereof (e.g. an H-chain, such as an H_{C} or H_{CC} domain thereof) bound to the capture substrates is the same as the heavy-chain binding positive control, it may be determined that the clostridial neurotoxin polypeptides or a portion thereof (e.g. an H-chain, such as an H_{C} or H_{CC} domain thereof) comprised in the composition do not comprise an activity-altering property of the H-chain.

The amount of clostridial neurotoxin polypeptides or a portion thereof (e.g. an H-chain, such as an H_{C} or H_{CC} domain thereof) bound to the capture substrates may be determined using any suitable method, such as one described hereinabove.

Any of the methods described herein may further determine whether or not clostridial neurotoxin polypeptides or a portion thereof (e.g. an H-chain, such as an H_{C} or H_{CC} domain thereof) comprised in the composition comprise an activity-altering property.

The method may further comprise: (i) adding a reducing agent for dissociating light-chain (L-chain) polypeptides of the bound clostridial neurotoxin polypeptides; and (ii) determining an amount of cleavable substrates cleaved by the L-chain polypeptides, thereby determining the clostridial neurotoxin activity of the composition.

The method may further comprise: (i) adding a reducing agent for dissociating light-chain (L-chain) polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and (ii) determining an amount of cleavable substrates cleaved in the assay sample by the L-chain polypeptides, thereby determining the clostridial neurotoxin activity of the composition.

In one embodiment when a method comprises the use of capture substrates (e.g. comprising an extracellular portion of a modified human SYT-II), said methods may comprise comparing the amount of cleavable substrates cleaved by the L-chain polypeptides with a control.

A suitable control may be any control described herein. A negative control may be representative of a composition comprising clostridial neurotoxin polypeptides that comprise an activity-decreasing property.

The control is preferably a positive control, more preferably a substrate cleavage positive control as described herein.

In one embodiment, when the clostridial neurotoxin activity of the composition is lower than the substrate cleavage positive control, it may be determined that the clostridial neurotoxin polypeptides comprised in the composition comprise an activity-decreasing property of the L-chain.

In one embodiment, when the clostridial neurotoxin activity of the composition is higher than the substrate cleavage positive control, it may be determined that the clostridial neurotoxin polypeptides comprised in the composition comprise an activity-increasing property of the L-chain.

In one embodiment, when a the clostridial neurotoxin activity of the composition is the same as the substrate cleavage positive control, it may be determined that the clostridial neurotoxin polypeptides comprised in the composition do not comprise an activity-altering property of the L-chain.

In one aspect, the invention provides a method for producing a therapeutic or cosmetic clostridial neurotoxin composition, the method comprising:
(a) obtaining the results of a method according to the invention; and
(b) formulating and/or packaging the composition for therapeutic or cosmetic use when the clostridial neurotoxin polypeptides comprised in the composition do not comprise the activity-altering property; or
(c) subjecting the composition to further purification when the clostridial neurotoxin polypeptides comprised in the composition do comprise the activity-altering property; and
(d) formulating and/or packaging the further purified composition for therapeutic or cosmetic use.

In one embodiment, where the term “obtaining the result” or “obtaining the results” of a method or assay is recited herein, the method carried out to obtain said result(s) may be carried out as part of a method of the invention.

### In One Aspect, the Invention Provides a Kit Comprising:

(a) an isolated capture substrate described herein; and
(b) optionally means for detecting binding of a botulinum neurotoxin to the capture substrate; and/or
(c) optionally instructions for the use of the same.

Embodiments related to the various methods of the invention are intended to be applied equally to alternative methods, products, and/or uses, and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein.

Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262 (5131) Science 208-214 (1993); Align-M, see, e.g., Ivo Van Walle et al., Align-M-A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a SEQ ID NO described herein to define amino acid position numbering, as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

A R N D C Q E G H I L K M F P S T W Y V
A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

Total number of identical matches×100
[length of the longer sequence plus the number of gaps introduced into the longer sequence in order to align the two sequences]

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows a schematic diagram of an example assay. BoNT (¾ circle) is bound by a capture substrate (triangle), following suitable wash steps, dithiothreitol (DTT) is added to reduce the disulphide bond between the BoNT H-chain and L-chain, thereby liberating the L-chain. The L-chain polypeptides are removed to a separate vial (e.g. well) and incubated with cleavable substrates comprising SNAP-25 flanked by cyan fluorescent protein (CFP) and yellow fluorescent protein (YFP). The CFP/YFP ratio is measured to determine activity.

FIG. 2 shows toxin binding by way of an increased optical density (OD) value when casein or 1% bovine serum albumin (BSA) were used as carrier protein blocking buffers.

FIG. 3 shows binding of mrBoNT/A to either SV2c capture substrates alone or SV2c and GT1b.

FIG. 4 shows the amount of SNAP-25 cleaved (represented by a decreased CFP/YFP ratio) in an assay according to FIG. 1, when the tested composition contains full-length mrBoNT/A (circle) or L-chain only (square).

FIG. 5 shows assay qualification of both mrBoNT/A (A) and mrBoNT/AB (B) demonstrating linearity, accuracy and precision between 150% and 50% levels.

FIG. 6 shows % activity of an mrBoNT/A composition following forced oxidation for a time indicated on the x-axis when tested in a cell-based assay (circle), endopeptidase assay (triangle), ELISA (inverted triangle), or a binding and cleavage assay as per FIG. 1 (diamond). Also presented is the amount of oxidised (specifically the % of unmodified/non-oxidised) mrBoNT/A peptide over time encompassing at least a portion of the H_{C} domain (square).

FIG. 7 shows a bi-layer interferometry (BLI) binding kinetic summary of non-oxidised (control) or oxidised (48 hours 0.01% H₂O₂) mrBoNT/A to capture substrates comprising an E. coli-expressed extracellular portion of non-glycosylated GST-SV2c (upper panels) or capture substrates comprising an extracellular portion of glycosylated HEK293-expressed GST-SV2c (lower panels).

FIG. 8 shows % activity of an mrBoNT/AB composition when tested in a cell-based assay (triangle) or an ELISA using capture substrates comprising an extracellular portion of an L51F mutated human SYTII (circle). Also presented is the amount of oxidised (specifically the % of unchanged/non-oxidised) mrBoNT/AB peptide encompassing at least a portion of the H_{C} domain (square).

FIG. 9 shows a bi-layer interferometry (BLI) binding kinetic summary of non-oxidised (control) or oxidised (72 hours 0.001% H₂O₂) mrBoNT/AB to capture substrates comprising an extracellular portion of an L51F mutated human SYTII capture substrate.

FIG. 10 shows a correlation between BoNT activity results for a cell-free assay according to FIG. 1 compared to a cell-based assay.

FIG. 11 shows: (A) a cleavable substrate having: a first luciferase domain; a linker comprising a SNAP-25 cleavage site flanked by spacers; and a second luciferase domain; and (B) a schematic diagram of an example assay. BoNT (¾ circle) is bound by a capture substrate (triangle), following suitable wash steps, dithiothreitol (DTT) and cleavable substrates are added to reduce the disulphide bond between the BoNT H-chain and L-chain, thereby liberating the L-chain. The L-chain is not removed. Subsequently, a luciferase substrate is added and luminescence determined to calculate BoNT activity of the composition.

FIG. 12 shows: (A) BoNT activity of a composition when assessed using cleavable substrates shown in FIG. 1 (comprising CFP and YFP); (B) BoNT activity of the same composition when assessed using cleavable substrates shown in FIG. 11A (comprising first and second luciferase domains) but where following DTT reduction, the L-chain polypeptides are removed to a separate vial (e.g. well) and incubated with the cleavable substrates; and (C) BoNT activity of the same composition when assessed using the method shown in FIG. 11B (i.e. where the L-chain is not removed cleavable substrates according to FIG. 11A are added to the vial (e.g. well)).

FIG. 13 shows assay qualification of mrBoNT/AB method shown in FIG. 11B demonstrating linearity, accuracy and precision between 150% and 50% levels.

FIG. 14 shows: (A) a reference curve generated when assessed in accordance with FIG. 1 but using cleavable substrates shown in FIG. 11A (comprising first and second luciferase domains); and (B) 5 ng/ml mrBoNT/AB targeted recovery in drug product buffer replicating increasing reconstitution in saline.

FIG. 15 shows linearity and parallelism of drug product assay variation between 130% and 75% levels.

FIG. 16 shows combined results for various assay formats for forced degraded samples of mrBoNT/A (A) and mrBoNT/AB (48 hr 0.001% H₂O₂) (B), compared to cell based assay data (control).

FIG. 17 shows the % activity of oxidised mrBoNT/AB in a modified binding and cleavage assay (see Example 2) employing either human SYT-I (squares) or human SYT-II (L51F modified-circles) capture substrates. The activity is shown versus the % of clostridial neurotoxin polypeptides in the composition oxidised at a reference amino acid residue.

### SEQUENCE LISTING

Where an initial Met amino acid residue or a corresponding initial codon is indicated in any of the following SEQ ID NOs, said residue/codon is optional. Preferably, said initial Met amino acid residue or corresponding initial codon is absent.

SEQ ID NO: 1-Polypeptide Sequence of Luciferase
MVFTLEDFVGDWEQTAAYNLDQVLEQGGVSSLLQNLAVSVTPIQRIVRSGENALKIDIHVIIP
YEGLSADQMAQIEEVFKVVYPVDDHHFKVILPYGTLVIDGVTPNMLNYFGRPYEGIAVFDGK
KITVTGTLWNGNKIIDERLITPDGSMLFRVTINSVTGYRLFEEIL
SEQ ID NO: 2-Polypeptide Sequence of Luciferase Domain 1
MVFTLEDFVGDWEQTAAYNLDQVLEQGGVSSLLQNLAVSVTPIQRIVRSGENALKIDIHVIIP
YEGLSADQMAQIEEVFKVVYPVDDHHFKVILPYGTLVIDGVTPNMLNYFGRPYEGIAVFDGK
KITVTGTLWNGNKIIDERLITPDGSMLFRVTINS
SEQ ID NO: 3-Polypeptide Sequence of Luciferase Domain 2
VTGYRLFEEIL
SEQ ID NO: 4 Polypeptide Sequence of Full-Length SNAP-25
MAEDADMRNELEEMQRRADQLADESLESTRRMLQLVEESKDAGIRTLVMLDEQGEQLERI
EEGMDQINKDMKEAEKNLTDLGKFCGLCVCPCNKLKSSDAYKKAWGNNQDGVVASQPAR
WVDEREQMAISGGFIRRVTNDARENEMDENLEQVSGIIGNLRHMALDMGNEIDTQNRQIDRI
MEKADSNKTRIDEANQRATKMLGSG
SEQ ID NO: 5-Polypeptide Sequence of 65 Amino Acid SNAP-25
RENEMDENLEQVSGIIGNLRHMALDMGNEIDTQNRQIDRIMEKADSNKTRIDEANQRAT
KMLGSG
SEQ ID NO: 6-Polypeptide Sequence of Luciferase Cleavable Substrate
MVFTLEDFVGDWEQTAAYNLDQVLEQGGVSSLLQNLAVSVTPIQRIVRSGENALKIDIHVIIP
YEGLSADQMAQIEEVFKVVYPVDDHHFKVILPYGTLVIDGVTPNMLNYFGRPYEGIAVFDGK
KITVTGTLWNGNKIIDERLITPDGSMLFRVTINSSGGGGSRENEMDENLEQVSGIIGNLRHMA
LDMGNEIDTQNRQIDRIMEKADSNKTRIDEANQRATKMLGSGSGGGGSVTGYRLFEEIL
SEQ ID NO: 7-Polypeptide Sequence of mrBoNT/AB
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNILNNIILNLRYKDNNLIDLSGYG
AKVEVYDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVTITNNLN
NAKIYINGKLESNTDIKDIREVIANGEIIFKLDGDIDRTQFIWMKYFSIFNTELSQSNIEERYKIQS
YSEYLKDFWGNPLMYNKEYYMFNAGNKNSYIKLKKDSPVGEILTRSKYNQNSKYINYRDLYI
GEKFIIRRKSNSQSINDDIVRKEDYIYLDFFNLNQEWRVYTYKYFKKEEMKLFLAPIYDSDEFY
NTIQIKEYDEQPTYSCQLLFKKDEESTDEIGLIGIHRFYESGIVFEEYKDYFCISKWYLKEVKR
KPYNLKLGCNWQFIPKDEGWTE
SEQ ID NO: 8-Polypeptide Sequence of BoNT/AB Variant 2
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKSEILNNIILNLRYKDNNLIDLSGYGAKVE
VYDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVT
ITNNLNNAKIYINGKLESNTDIKDIREVIANGEIIFKLDGDIDRTQFIWMKYFSIFNTEL
SQSNIEERYKIQSYSEYLKDFWGNPLMYNKEYYMFNAGNKNSYIKLKKDSPVGEILTRSK
YNQNSKYINYRDLYIGEKFIIRRKSNSQSINDDIVRKEDYIYLDFFNLNQEWRVYTYKYF
KKEEMKLFLAPIYDSDEFYNTIQIKEYDEQPTYSCQLLFKKDEESTDEIGLIGIHRFYES
GIVFEEYKDYFCISKWYLKEVKRKPYNLKLGCNWQFIPKDEGWTEHHHHHHHHHH
SEQ ID NO: 9-Polypeptide Sequence of BoNT/AB Variant 3
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIIELGGGGSELSEILNNIILNLRYKDNN
LIDLSGYGAKVEVYDGVELNDKNQFKLTSSANSKIRVTQNQNIIFNSVFLDFSVSFWIRI
PKYKNDGIQNYIHNEYTIINCMKNNSGWKISIRGNRIIWTLIDINGKTKSVFFEYNIRED
ISEYINRWFFVTITNNLNNAKIYINGKLESNTDIKDIREVIANGEIIFKLDGDIDRTQFI
WMKYFSIFNTELSQSNIEERYKIQSYSEYLKDFWGNPLMYNKEYYMFNAGNKNSYIKLKK
DSPVGEILTRSKYNQNSKYINYRDLYIGEKFIIRRKSNSQSINDDIVRKEDYIYLDFFNL
NQEWRVYTYKYFKKEEMKLFLAPIYDSDEFYNTIQIKEYDEQPTYSCQLLFKKDEESTDE
IGLIGIHRFYESGIVFEEYKDYFCISKWYLKEVKRKPYNLKLGCNWQFIPKDEGWTEHHH
HHHHHHH
SEQ ID NO: 10-Polypeptide Sequence of BoNT/AB Variant 4
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTEHHHHHHHHHH
SEQ ID NO: 11-Polypeptide Sequence of BoNT/AB Variant 5
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEEKLFLAPISDSDEFYNTIQIKEYDEQPTYSCQLLFKKDEESTDEIGLIGIHRFYESG
IVFEEYKDYFCISKWYLKEVKRKPYNLKLGCNWQFIPKDEGWTE
SEQ ID NO: 12-Polypeptide Sequence of Native BoNT/A (BoNT/A)
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSISLNNEY
TIINCMENNSGWKVSLNYGEIIWTLQDTQEIKQRVVFKYSQMINISDYINRWIFVTITNNRLNN
SKIYINGRLIDQKPISNLGNIHASNNIMFKLDGCRDTHRYIWIKYFNLFDKELNEKEIKDLYDNQ
SNSGILKDFWGDYLQYDKPYYMLNLYDPNKYVDVNNVGIRGYMYLKGPRGSVMTTNIYLNS
SLYRGTKFIIKKYASGNKDNIVRNNDRVYINVVVKNKEYRLATNASQAGVEKILSALEIPDVGN
LSQVVVMKSKNDQGITNKCKMNLQDNNGNDIGFIGFHQFNNIAKLVASNWYNRQIERSSRT
LGCSWEFIPVDDGWGERPL
SEQ ID NO: 13-Polypeptide Sequence of BoNT/B
MPVTINNFNYNDPIDNNNIIMMEPPFARGTGRYYKAFKITDRIWIIPERYTFGYKPEDFN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVFNNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPFDL
SIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 14-Polypeptide Sequence of mrBoNT/A
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNIINTSILNLRYESKHLIDLSRYAS
KINIGSKVNFDPIDKNQIQLFNLESSKIEVILKKAIVYNSMYENFSTSFWIRIPKYFNKISLNNEY
TIINCMENNSGWKVSLNYGEIIWTLQDTKEIKQRVVFKYSQMINISDYINRWIFVTITNNRLNK
SKIYINGRLIDQKPISNLGNIHASNKIMFKLDGCRDTHRYIWIKYFNLFDKELNEKEIKDLYDNQ
SNSGILKDFWGDYLQYDKPYYMLNLYDPNKYVDVNNVGIRGYMYLKGPRGSVMTTNIYLNS
SLYRGTKFIIKKYASGNKDNIVRNNDRVYINVVVKNKEYRLATNASQAGVEKILSALEIPDVGN
LSQVVVMKSKNDKGITNKCKMNLQDNNGNDIGFIGFHQFNNIAKLVASNWYNRQIERSSRTL
GCSWEFIPVDDGWGERPL
SEQ ID NO: 15-Polypeptide Sequence of Cationic BoNT/A Variant 2
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNIINTSILNLRYESNHLIDLSRYAS
KINIGSKVNFDPIDKNQIQLFNLESSKIEVILKKAIVYNSMYENFSTSFWIRIPKYFKKISLNNEY
TIINCMENNSGWKVSLNYGEIIWTLQDTKEIKQRVVFKYSQMINISDYINRWIFVTITNNRLNK
SKIYINGRLIDQKPISNLGNIHASNKIMFKLDGCRDTHRYIWIKYFNLFDKELNEKEIKDLYDNQ
SNSGILKDFWGDYLQYDKPYYMLNLYDPNKYVDVNNVGIRGYMYLKGPRGSVMTTNIYLNS
SLYRGTKFIIKKYASGNKDNIVRNNDRVYINVVVKNKEYRLATNASQAGVEKILSALEIPDVGN
LSQVVVMKSKNDKGITNKCKMNLQDNNGNDIGFIGFHQFNNIAKLVASNWYNRQIERSSRTL
GCSWEFIPVDDGWGERPL
SEQ ID NO: 16-Polypeptide Sequence of Cationic BoNT/A Variant 3
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNIINTSILNLRYESNHLIDLSRYAS
KINIGSKVNFDPIDKNQIQLFNLESSKIEVILKKAIVYNSMYENFSTSFWIRIPKYFNKISLNNEY
TIINCMENNSGWKVSLNYGEIIWTLQDTKEIKQRVVFKYSQMINISDYINRWIFVTITNNRLKKS
KIYINGRLIDQKPISNLGNIHASNKIMFKLDGCRDTHRYIWIKYFNLFDKELNEKEIKDLYDNQS
NSGILKDFWGDYLQYDKPYYMLNLYDPNKYVDVNNVGIRGYMYLKGPRGSVMTTNIYLNSS
LYRGTKFIIKKYASGNKDNIVRNNDRVYINVVVKNKEYRLATNASQAGVEKILSALEIPDVGNL
SQVVVMKSKNDKGITNKCKMNLQDNNGNDIGFIGFHQFNNIAKLVASNWYNRQIERSSRTL
GCSWEFIPVDDGWGERPL
SEQ ID NO: 17-Polypeptide Sequence of Cationic BoNT/A Variant 4
MPFVNKQFNYKDPVNGVDIAYIKIPNAGQMQPVKAFKIHNKIWVIPERDTFTNPEEGDLNPPP
EAKQVPVSYYDSTYLSTDNEKDNYLKGVTKLFERIYSTDLGRMLLTSIVRGIPFWGGSTIDTE
LKVIDTNCINVIQPDGSYRSEELNLVIIGPSADIIQFECKSFGHEVLNLTRNGYGSTQYIRFSPD
FTFGFEESLEVDTNPLLGAGKFATDPAVTLAHELIHAGHRLYGIAINPNRVFKVNTNAYYEMS
GLEVSFEELRTFGGHDAKFIDSLQENEFRLYYYNKFKDIASTLNKAKSIVGTTASLQYMKNVF
KEKYLLSEDTSGKFSVDKLKFDKLYKMLTEIYTEDNFVKFFKVLNRKTYLNFDKAVFKINIVPK
VNYTIYDGFNLRNTNLAANFNGQNTEINNMNFTKLKNFTGLFEFYKLLCVRGIITSKTKSLDK
GYNKALNDLCIKVNNWDLFFSPSEDNFTNDLNKGEEITSDTNIEAAEENISLDLIQQYYLTFNF
DNEPENISIENLSSDIIGQLELMPNIERFPNGKKYELDKYTMFHYLRAQEFEHGKSRIALTNSV
NEALLNPSRVYTFFSSDYVKKVNKATEAAMFLGWVEQLVYDFTDETSEVSTTDKIADITIIIPYI
GPALNIGNMLYKDDFVGALIFSGAVILLEFIPEIAIPVLGTFALVSYIANKVLTVQTIDNALSKRN
EKWDEVYKYIVTNWLAKVNTQIDLIRKKMKEALENQAEATKAIINYQYNQYTEEEKNNINFNID
DLSSKLNESINKAMININKFLNQCSVSYLMNSMIPYGVKRLEDFDASLKDALLKYIYDNRGTLI
GQVDRLKDKVNNTLSTDIPFQLSKYVDNQRLLSTFTEYIKNIINTSILNLRYESNHLIDLSRYAS
KINIGSKVNFDPIDKNQIQLFNLESSKIEVILKNAIVYNSMYENFSTSFWIRIPKYFNSISLNNEY
TIINCMENNSGWKVSLNYGEIIWTLQDTQEIKQRVVFKYSQMINISDYINRWIFVTITNNRLNN
SKIYINGRLIDQKPISNLGNIHASNNIMFKLDGCRDTHRYIWIKYFNLFDKELNEKEIKDLYDNQ
SNSGILKDFWGDYLQYDKPYYMLNLYDPNKYVDVNNVGIRGYMYLKGPRGSVMTTNIYLNS
SLYRGTKFIIKKYASGNKDNIVRNNDRVYINVVVKRKEYRLATNASQAGVEKILSALEIPRVRR
LSQVVVMKSKNDQGITNKCKMNLQDRRGNDIGFIGFHQFNNIAKLVASNWYNRQIERRSRR
LGCSWEFIPVDDGWGERPL
SEQ ID NO: 18-Polypeptide Sequence of Capture Substrate Comprising an
Extracellular Portion (Amino Acids 1-61) of Human Wild-type SYTII
MSGSHHHHHHSSGMSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMCLDAF
PKLVCFKKRIEAIPQIDKYLKSSKYIAWPLQGWQATFGGGDHPPKSDLGHTGHRSGTENLYF
QGMRNIFKRNQEPIVAPATTTATMPIGPVDNSTESGGAGESQEDMFAKLKEKLFNEINKIPLP
SEQ ID NO: 19-Polypeptide Sequence of an Extracellular Portion (Amino Acids 1-61)
of Human Wild-type SYTII
MRNIFKRNQEPIVAPATTTATMPIGPVDNSTESGGAGESQEDMFAKLKEKLFNEINKIPLP
SEQ ID NO: 20-Polypeptide Sequence of Capture Substrate Comprising an
Extracellular Portion (Amino Acids 1-61) of Human Modified SYTII (L51F)
MSGSHHHHHHSSGMSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMCLDAF
PKLVCFKKRIEAIPQIDKYLKSSKYIAWPLQGWQATFGGGDHPPKSDLGHTGHRSGTENLYF
QGMRNIFKRNQEPIVAPATTTATMPIGPVDNSTESGGAGESQEDMFAKLKEKFFNEINKIPLP
SEQ ID NO: 21-Polypeptide Sequence of an Extracellular Portion (Amino Acids 1-61)
of Human Modified SYTII (L51F)
MRNIFKRNQEPIVAPATTTATMPIGPVDNSTESGGAGESQEDMFAKLKEKFFNEINKIPLP
SEQ ID NO: 22-Polypeptide Sequence of Capture Substrate Comprising an
Extracellular Portion (Amino Acids 1-64) of Mouse SYTII
MSGSHHHHHHSSGMSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMCL
DAFPKLVCFKKRIEAIPQIDKYLKSSKYIAWPLQGWQATFGGGDHPPKSDLGHTGHRSGTE
NLYFQGMRNIFKRNGEPNVAPATTTATMPLAPVAPADNSTESTGPGESQEDMFAKLKEKFF
NEINKIPLP
SEQ ID NO: 23-Polypeptide Sequence of Full-Length Human Wild-type SYTII
MRNIFKRNQEPIVAPATTTATMPIGPVDNSTESGGAGESQEDMFAKLKEKLFNEINKIPL
PPWALIAIAVVAGLLLLTCCFCICKKCCCKKKKNKKEKGKGMKNAMNMKDMKGGQDDDDA
ETGLTEGEGEGEEEKEPENLGKLQFSLDYDFQANQLTVGVLQAAELPALDMGGTSDPYVK
VFLLPDKKKKYETKVHRKTLNPAFNETFTFKVPYQELGGKTLVMAIYDFDRFSKHDIIGE
VKVPMNTVDLGQPIEEWRDLQGGEKEEPEKLGDICTSLRYVPTAGKLTVCILEAKNLKKM
DVGGLSDPYVKIHLMQNGKRLKKKKTTVKKKTLNPYFNESFSFEIPFEQIQKVQVVVTVL
DYDKLGKNEAIGKIFVGSNATGTELRHWSDMLANPRRPIAQWHSLKPEEEVDALLGKNK
SEQ ID NO: 24-Polypeptide Sequence of Full-Length Mouse Wild-type SYTII
MRNIFKRNQEPNVAPATTTATMPLAPVAPADNSTESTGPGESQEDMFAKLKEKFFNEINK
IPLPPWALIAMAVVAGLLLLTCCFCICKKCCCKKKKNKKEKGKGMKNAMNMKDMKGGQDD
DDAETGLTEGEGEGEEEKEPENLGKLQFSLDYDFQANQLTVGVLQAAELPALDMGGTSDP
YVKVFLLPDKKKKYETKVHRKTLNPAFNETFTFKVPYQELAGKTLVMAIYDFDRFSKHDI
IGEVKVPMNTVDLGQPIEEWRDLQGGEKEEPEKLGDICTSLRYVPTAGKLTVCILEAKNL
KKMDVGGLSDPYVKIHLMQNGKRLKKKKTTVKKKTLNPYFNESFSFEIPFEQIQKVQVVV
TVLDYDKLGKNEAIGKIFVGSNATGTELRHWSDMLANPRRPIAQWHSLKPEEEVDALLGK
NK
SEQ ID NO: 25-Polypeptide Sequence of Full-Length Human SV2a
MEEGFRDRAAFIRGAKDIAKEVKKHAAKKVVKGLDRVQDEYSRRSYSRFEEEDDDDDFPA
PSDGYYRGEGTQDEEEGGASSDATEGHDEDDEIYEGEYQGIPRAESGGKGERMADGAPL
A
GVRGGLSDGEGPPGGRGEAQRRKEREELAQQYEAILRECGHGRFQWTLYFVLGLALMAD
G
VEVFVVGFVLPSAEKDMCLSDSNKGMLGLIVYLGMMVGAFLWGGLADRLGRRQCLLISLS
VNSVFAFFSSFVQGYGTFLFCRLLSGVGIGGSIPIVFSYFSEFLAQEKRGEHLSWLCMFW
MIGGVYAAAMAWAIIPHYGWSFQMGSAYQFHSWRVFVLVCAFPSVFAIGALTTQPESPRF
FLENGKHDEAWMVLKQVHDTNMRAKGHPERVFSVTHIKTIHQEDELIEIQSDTGTWYQRW
GVRALSLGGQVWGNFLSCFGPEYRRITLMMMGVWFTMSFSYYGLTVWFPDMIRHLQAVD
Y
ASRTKVFPGERVEHVTFNFTLENQIHRGGQYFNDKFIGLRLKSVSFEDSLFEECYFEDVT
SSNTFFRNCTFINTVFYNTDLFEYKFVNSRLINSTFLHNKEGCPLDVTGTGEGAYMVYFV
SFLGTLAVLPGNIVSALLMDKIGRLRMLAGSSVMSCVSCFFLSFGNSESAMIALLCLFGG
VSIASWNALDVLTVELYPSDKRTTAFGFLNALCKLAAVLGISIFTSFVGITKAAPILFAS
AALALGSSLALKLPETRGQVLQ
SEQ ID NO: 26-Polypeptide Sequence of Full-Length Human SV2b
MDDYKYQDNYGGYAPSDGYYRGNESNPEEDAQSDVTEGHDEEDEIYEGEYQGIPHPDDV
K
AKQAKMAPSRMDSLRGQTDLMAERLEDEEQLAHQYETIMDECGHGRFQWILFFVLGLALM
ADGVEVFVVSFALPSAEKDMCLSSSKKGMLGMIVYLGMMAGAFILGGLADKLGRKRVLSM
SLAVNASFASLSSFVQGYGAFLFCRLISGIGIGGALPIVFAYFSEFLSREKRGEHLSWLG
IFWMTGGLYASAMAWSIIPHYGWGFSMGTNYHFHSWRVFVIVCALPCTVSMVALKFMPES
PRFLLEMGKHDEAWMILKQVHDTNMRAKGTPEKVFTVSNIKTPKQMDEFIEIQSSTGTWY
QRWLVRFKTIFKQVWDNALYCVMGPYRMNTLILAVVWFAMAFSYYGLTVWFPDMIRYFQD
EEYKSKMKVFFGEHVYGATINFTMENQIHQHGKLVNDKFTRMYFKHVLFEDTFFDECYFE
DVTSTDTYFKNCTIESTIFYNTDLYEHKFINCRFINSTFLEQKEGCHMDLEQDNDFLIYL
VSFLGSLSVLPGNIISALLMDRIGRLKMIGGSMLISAVCCFFLFFGNSESAMIGWQCLFC
GTSIAAWNALDVITVELYPTNQRATAFGILNGLCKFGAILGNTIFASFVGITKVVPILLA
AASLVGGGLIALRLPETREQVLM
SEQ ID NO: 27-Polypeptide Sequence of Full-Length Human SV2c
MEDSYKDRTSLMKGAKDIAREVKKQTVKKVNQAVDRAQDEYTQRSYSRFQDEEDDDDYYP
AGETYNGEANDDEGSSEATEGHDEDDEIYEGEYQGIPSMNQAKDSIVSVGQPKGDEYKDR
RELESERRADEEELAQQYELIIQECGHGRFQWALFFVLGMALMADGVEVFVVGFVLPSAE
TDLCIPNSGSGWLGSIVYLGMMVGAFFWGGLADKVGRKQSLLICMSVNGFFAFLSSFVQG
YGFFLFCRLLSGFGIGGAIPTVFSYFAEVLAREKRGEHLSWLCMFWMIGGIYASAMAWAI
IPHYGWSFSMGSAYQFHSWRVFVIVCALPCVSSVVALTFMPESPRFLLEVGKHDEAWMIL
KLIHDTNMRARGQPEKVFTVNKIKTPKQIDELIEIESDTGTWYRRCFVRIRTELYGIWLT
FMRCFNYPVRDNTIKLTIVWFTLSFGYYGLSVWFPDVIKPLQSDEYALLTRNVERDKYAN
FTINFTMENQIHTGMEYDNGRFIGVKFKSVTFKDSVFKSCTFEDVTSVNTYFKNCTFIDT
VFDNTDFEPYKFIDSEFKNCSFFHNKTGCQITFDDDYSAYWIYFVNFLGTLAVLPGNIVS
ALLMDRIGRLTMLGGSMVLSGISCFFLWFGTSESMMIGMLCLYNGLTISAWNSLDVVTVE
LYPTDRRATGFGFLNALCKAAAVLGNLIFGSLVSITKSIPILLASTVLVCGGLVGLCLPD
TRTQVLM
SEQ ID NO: 28-Polypeptide Sequence of Full-Length Human SYT-I
MVSESHHEALAAPPVTTVATVLPSNATEPASPGEGKEDAFSKLKEKFMNELHKIPLPPWA
LIAIAIVAVLLVLTCCFCICKKCLFKKKNKKKGKEKGGKNAINMKDVKDLGKTMKDQALK
DDDAETGLTDGEEKEEPKEEEKLGKLQYSLDYDFQNNQLLVGIIQAAELPALDMGGTSDP
YVKVFLLPDKKKKFETKVHRKTLNPVFNEQFTFKVPYSELGGKTLVMAVYDFDRFSKHDI
IGEFKVPMNTVDFGHVTEEWRDLQSAEKEEQEKLGDICFSLRYVPTAGKLTVVILEAKNL
KKMDVGGLSDPYVKIHLMQNGKRLKKKKTTIKKNTLNPYYNESFSFEVPFEQIQKVQVVV
TVLDYDKIGKNDAIGKVFVGYNSTGAELRHWSDMLANPRRPIAQWHTLQVEEEVDAMLAV
KK
SEQ ID NO: 29-Polypeptide Sequence of Capture Substrate Comprising an
Extracellular Portion (Amino Acids 473 to 567) of SV2c
MSPILGYWKIKGLVQPTRLLLEYLEEKYEEHLYERDEGDKWRNKKFELGLEFPNLPYYIDGD
VKLTQSMAIIRYIADKHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPKLVCFKKRIEAIP
QIDKYLKSSKYIAWPLQGWQATFGGGDHPPKSDLSSGLEVLFQGPVERDKYANFTINFTME
NQIHTGMEYDNGRFIGVKFKSVTFKDSVFKSCTFEDVTSVNTYFKNCTFIDTVFDNTDFEPY
KFIDSEFKNCSFFHNKT
SEQ ID NO: 30-Polypeptide Sequence of an Extracellular Portion (Amino Acids 473 to
567) of SV2c
VERDKYANFTINFTMENQIHTGMEYDNGRFIGVKFKSVTFKDSVFKSCTFEDVTSVNTYFKN
CTFIDTVFDNTDFEPYKFIDSEFKNCSFFHNKT
SEQ ID NO: 31-Polypeptide Sequence of VAMP1 human (P23763)
MSAPAQPPAEGTEGTAPGGGPPGPPPNMTSNRRLQQTQAQVEEVVDIIRVNVDKVLERDQ
KLSELDDRADALQAGASQFESSAAKLKRKYWWKNCKMMIMLGAICAIIVVVIVIYFFT
SEQ ID NO: 32-Polypeptide Sequence of VAMP2 human (P63027)
MSATAATAPPAAPAGEGGPPAPPPNLTSNRRLQQTQAQVDEVVDIMRVNVDKVLERDQKL
SELDDRADALQAGASQFETSAAKLKRKYWWKNLKMMIILGVICAIILIIIIVYFST
SEQ ID NO: 33-Polypeptide Sequence of VAMP3 human (Q15836)
MSTGPTAATGSNRRLQQTQNQVDEVVDIMRVNVDKVLERDQKLSELDDRADALQAGASQF
ETSAAKLKRKYWWKNCKMWAIGITVLVIFIIIIIVWVVSS
SEQ ID NO: 34-Polypeptide Sequence of VAMP4 human (O75379)
MPPKFKRHLNDDDVTGSVKSERRNLLEDDSDEEEDFFLRGPSGPRFGPRNDKIKHVQNQV
DEVIDVMQENITKVIERGERLDELQDKSESLSDNATAFSNRSKQLRRQMWWRGCKIKAIMAL
VAAILLLVIIILIVMKYRT
SEQ ID NO: 35-Polypeptide Sequence of VAMP5 human (O95183)
MAGIELERCQQQANEVTEIMRNNFGKVLERGVKLAELQQRSDQLLDMSSTFNKTTQNLAQK
KCWENIRYRICVGLVVVGVLLIILIVLLVVFLPQSSDSSSAPRTQDAGIASGPGN
SEQ ID NO: 36-Polypeptide Sequence of YKT6 human (O15498)
MKLYSLSVLYKGEAKVVLLKAAYDVSSFSFFQRSSVQEFMTFTSQLIVERSSKGTRASVKEQ
DYLCHVYVRNDSLAGVVIADNEYPSRVAFTLLEKVLDEFSKQVDRIDWPVGSPATIHYPALD
GHLSRYQNPREADPMTKVQAELDETKIILHNTMESLLERGEKLDDLVSKSEVLGTQSKAFYK
TARKQNSCCAIM
SEQ ID NO: 37-Polypeptide Sequence of Syntaxin 1A
MKDRTQELRTAKDSDDDDDVAVTVDRDRFMDEFFEQVEEIRGFIDKIAENVEEVKRKHSA
ILASPNPDEKTKEELEELMSDIKKTANKVRSKLKSIEQSIEQEEGLNRSSADLRIRKTQH
STLSRKFVEVMSEYNATQSDYRERCKGRIQRQLEITGRTTTSEELEDMLESGNPAIFASG
IIMDSSISKQALSEIETRHSEIIKLENSIRELHDMFMDMAMLVESQGEMIDRIEYNVEHA
VDYVERAVSDTKKAVKYQSKARRKKIMIIICCVILGIVIASTVGGIFA
SEQ ID NO: 38-Polypeptide Sequence of Syntaxin 1B
MKDRTQELRSAKDSDDEEEVVHVDRDHFMDEFFEQVEEIRGCIEKLSEDVEQVKKQHSAI
LAAPNPDEKTKQELEDLTADIKKTANKVRSKLKAIEQSIEQEEGLNRSSADLRIRKTQHS
TLSRKFVEVMTEYNATQSKYRDRCKDRIQRQLEITGRTTTNEELEDMLESGKLAIFTDDI
KMDSQMTKQALNEIETRHNEIIKLETSIRELHDMFVDMAMLVESQGEMIDRIEYNVEHSV
DYVERAVSDTKKAVKYQSKARRKKIMIIICCVVLGVVLASSIGGTLGL
SEQ ID NO: 39-Polypeptide Sequence of Spacer
SGGGGS
SEQ ID NO: 40-Polypeptide Sequence of Capture Substrate Comprising an
Extracellular Portion (Amino Acids 473 to 567) of SV2c
MGWSCIILFLVATATGVHSGGGGSSPILGYWKIKGLVQPTRLLLEYLEEKYEEHLYERDEGD
KWRNKKFELGLEFPNLPYYIDGDVKLTQSMAIIRYIADKHNMLGGCPKERAEISMLEGAVLDI
RYGVSRIAYSKDFETLKVDFLSKLPEMLKMFEDRLCHKTYLNGDHVTHPDFMLYDALDVVLY
MDPMCLDAFPKLVCFKKRIEAIPQIDKYLKSSKYIAWPLQGWQATFGGGDHPPKSDLSSGLE
VLFQGPVERDKYANFTINFTMENQIHTGMEYDNGRFIGVKFKSVTFKDSVFKSCTFEDVTSV
NTYFKNCTFIDTVFDNTDFEPYKFIDSEFKNCSFFHNKT
SEQ ID NO: 41-Polypeptide Sequence of BoNT/C-UniProt P18640
MPITINNFNYSDPVDNKNILYLDTHLNTLANEPEKAFRITGNIWVIPDRFSRNSNPNLNK
PPRVTSPKSGYYDPNYLSTDSDKDPFLKEIIKLFKRINSREIGEELIYRLSTDIPFPGNN
NTPINTFDFDVDFNSVDVKTRQGNNWVKTGSINPSVIITGPRENIIDPETSTFKLTNNTF
AAQEGFGALSIISISPRFMLTYSNATNDVGEGRFSKSEFCMDPILILMHELNHAMHNLYG
IAIPNDQTISSVTSNIFYSQYNVKLEYAEIYAFGGPTIDLIPKSARKYFEEKALDYYRSI
AKRLNSITTANPSSFNKYIGEYKQKLIRKYRFVVESSGEVTVNRNKFVELYNELTQIFTE
FNYAKIYNVQNRKIYLSNVYTPVTANILDDNVYDIQNGFNIPKSNLNVLFMGQNLSRNPA
LRKVNPENMLYLFTKFCHKAIDGRSLYNKTLDCRELLVKNTDLPFIGDISDVKTDIFLRK
DINEETEVIYYPDNVSVDQVILSKNTSEHGQLDLLYPSIDSESEILPGENQVFYDNRTQN
VDYLNSYYYLESQKLSDNVEDFTFTRSIEEALDNSAKVYTYFPTLANKVNAGVQGGLFLM
WANDVVEDFTTNILRKDTLDKISDVSAIIPYIGPALNISNSVRRGNFTEAFAVTGVTILL
EAFPEFTIPALGAFVIYSKVQERNEIIKTIDNCLEQRIKRWKDSYEWMMGTWLSRIITQF
NNISYQMYDSLNYQAGAIKAKIDLEYKKYSGSDKENIKSQVENLKNSLDVKISEAMNNIN
KFIRECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEGDVQ
LNPIFPFDFKLGSSGEDRGKVIVTQNENIVYNSMYESFSISFWIRINKWVSNLPGYTIID
SVKNNSGWSIGIISNFLVFTLKQNEDSEQSINFSYDISNNAPGYNKWFFVTVTNNMMGNM
KIYINGKLIDTIKVKELTGINFSKTITFEINKIPDTGLITSDSDNINMWIRDFYIFAKEL
DGKDINILFNSLQYTNVVKDYWGNDLRYNKEYYMVNIDYLNRYMYANSRQIVFNTRRNNN
DFNEGYKIIIKRIRGNTNDTRVRGGDILYFDMTINNKAYNLFMKNETMYADNHSTEDIYA
IGLREQTKDINDNIIFQIQPMNNTYYYASQIFKSNFNGENISGICSIGTYRFRLGGDWYR
HNYLVPTVKQGNYASLLESTSTHWGFVPVSE
SEQ ID NO: 42-Polypeptide Sequence of BoNT/D-UniProt P19321
MTWPVKDFNYSDPVNDNDILYLRIPQNKLITTPVKAFMITQNIWVIPERFSSDTNPSLSK
PPRPTSKYQSYYDPSYLSTDEQKDTFLKGIIKLFKRINERDIGKKLINYLVVGSPFMGDS
STPEDTFDFTRHTTNIAVEKFENGSWKVTNIITPSVLIFGPLPNILDYTASLTLQGQQSN
PSFEGFGTLSILKVAPEFLLTFSDVTSNQSSAVLGKSIFCMDPVIALMHELTHSLHQLYG
INIPSDKRIRPQVSEGFFSQDGPNVQFEELYTFGGLDVEIIPQIERSQLREKALGHYKDI
AKRLNNINKTIPSSWISNIDKYKKIFSEKYNFDKDNTGNFVVNIDKFNSLYSDLTNVMSE
WVYSSQYNVKNRTHYFSRHYLPVFANILDDNIYTIRDGFNLTNKGFNIENSGQNIERNPA
LQKLSSESVVDLFTKVCLRLTKNSRDDSTCIKVKNNRLPYVADKDSISQEIFENKIITDE
TNVQNYSDKFSLDESILDGQVPINPEIVDPLLPNVNMEPLNLPGEEIVFYDDITKYVDYL
NSYYYLESQKLSNNVENITLTTSVEEALGYSNKIYTFLPSLAEKVNKGVQAGLFLNWANE
WVEDFTTNIMKKDTLDKISDVSVIIPYIGPALNIGNSALRGNFNQAFATAGVAFLLEGFP
EFTIPALGVFTFYSSIQEREKIIKTIENCLEQRVKRWKDSYQWMVSNWLSRITTQFNHIN
YQMYDSLSYQADAIKAKIDLEYKKYSGSDKENIKSQVENLKNSLDVKISEAMNNINKFIR
ECSVTYLFKNMLPKVIDELNKFDLRTKTELINLIDSHNIILVGEVDRLKAKVNESFENTM
PFNIFSYTNNSLLKDIINEYFNSINDSKILSLQNKKNALVDTSGYNAEVRVGDNVQLNTI
YTNDFKLSSSGDKIIVNLNNNILYSAIYENSSVSFWIKISKDLTNSHNEYTIINSIEQNS
GWKLCIRNGNIEWILQDVNRKYKSLIFDYSESLSHTGYTNKWFFVTITNNIMGYMKLYIN
GELKQSQKIEDLDEVKLDKTIVFGIDENIDENQMLWIRDFNIFSKELSNEDINIVYEGQI
LRNVIKDYWGNPLKFDTEYYIINDNYIDRYIAPESNVLVLVQYPDRSKLYTGNPITIKSV
SDKNPYSRILNGDNIILHMLYNSRKYMIIRDTDTIYATQGGECSQNCVYALKLQSNLGNY
GIGIFSIKNIVSKNKYCSQIFSSFRENTMLLADIYKPWRFSFKNAYTPVAVTNYETKLLS
TSSFWKFISRDPGWVE
SEQ ID NO: 43-Polypeptide Sequence of BoNT/E-UniProt Q00496
MPKINSFNYNDPVNDRTILYIKPGGCQEFYKSFNIMKNIWIIPERNVIGTTPQDFHPPTS
LKNGDSSYYDPNYLQSDEEKDRFLKIVTKIFNRINNNLSGGILLEELSKANPYLGNDNTP
DNQFHIGDASAVEIKFSNGSQDILLPNVIIMGAEPDLFETNSSNISLRNNYMPSNHRFGS
IAIVTFSPEYSFRFNDNCMNEFIQDPALTLMHELIHSLHGLYGAKGITTKYTITQKQNPL
ITNIRGTNIEEFLTFGGTDLNIITSAQSNDIYTNLLADYKKIASKLSKVQVSNPLLNPYK
DVFEAKYGLDKDASGIYSVNINKENDIFKKLYSFTEFDLRTKFQVKCRQTYIGQYKYFKL
SNLLNDSIYNISEGYNINNLKVNFRGQNANLNPRIITPITGRGLVKKIIRFCKNIVSVKG
IRKSICIEINNGELFFVASENSYNDDNINTPKEIDDTVTSNNNYENDLDQVILNFNSESA
PGLSDEKLNLTIQNDAYIPKYDSNGTSDIEQHDVNELNVFFYLDAQKVPEGENNVNLTSS
IDTALLEQPKIYTFFSSEFINNVNKPVQAALFVSWIQQVLVDFTTEANQKSTVDKIADIS
IVVPYIGLALNIGNEAQKGNFKDALELLGAGILLEFEPELLIPTILVFTIKSFLGSSDNK
NKVIKAINNALKERDEKWKEVYSFIVSNWMTKINTQFNKRKEQMYQALQNQVNAIKTIIE
SKYNSYTLEEKNELTNKYDIKQIENELNQKVSIAMNNIDRFLTESSISYLMKIINEVKIN
KLREYDENVKTYLLNYIIQHGSILGESQQELNSMVTDTLNNSIPFKLSSYTDDKILISYF
NKFFKRIKSSSVLNMRYKNDKYVDTSGYDSNININGDVYKYPTNKNQFGIYNDKLSEVNI
SQNDYIIYDNKYKNFSISFWVRIPNYDNKIVNVNNEYTIINCMRDNNSGWKVSLNHNEII
WTFEDNRGINQKLAFNYGNANGISDYINKWIFVTITNDRLGDSKLYINGNLIDQKSILNL
GNIHVSDNILFKIVNCSYTRYIGIRYFNIFDKELDETEIQTLYSNEPNTNILKDFWGNYL
LYDKEYYLLNVLKPNNFIDRRKDSTLSINNIRSTILLANRLYSGIKVKIQRVNNSSTNDN
LVRKNDQVYINFVASKTHLFPLYADTATTNKEKTIKISSSGNRFNQVVVMNSVGNCTMNF
KNNNGNNIGLLGFKADTVVASTWYYTHMRDHTNSNGCFWNFISEEHGWQEK
SEQ ID NO: 44-Polypeptide Sequence of BoNT/F-UniProt A7GBG3
MPVVINSFNYNDPVNDDTILYMQIPYEEKSKKYYKAFEIMRNVWIIPERNTIGTDPSDFD
PPASLENGSSAYYDPNYLTTDAEKDRYLKTTIKLFKRINSNPAGEVLLQEISYAKPYLGN
EHTPINEFHPVTRTTSVNIKSSTNVKSSIILNLLVLGAGPDIFENSSYPVRKLMDSGGVY
DPSNDGFGSINIVTFSPEYEYTFNDISGGYNSSTESFIADPAISLAHELIHALHGLYGAR
GVTYKETIKVKQAPLMIAEKPIRLEEFLTFGGQDLNIITSAMKEKIYNNLLANYEKIATR
LSRVNSAPPEYDINEYKDYFQWKYGLDKNADGSYTVNENKFNEIYKKLYSFTEIDLANKF
KVKCRNTYFIKYGFLKVPNLLDDDIYTVSEGFNIGNLAVNNRGQNIKLNPKIIDSIPDKG
LVEKIVKFCKSVIPRKGTKAPPRLCIRVNNRELFFVASESSYNENDINTPKEIDDTTNLN
NNYRNNLDEVILDYNSETIPQISNQTLNTLVQDDSYVPRYDSNGTSEIEEHNVVDLNVFF
YLHAQKVPEGETNISLTSSIDTALSEESQVYTFFSSEFINTINKPVHAALFISWINQVIR
DFTTEATQKSTFDKIADISLVVPYVGLALNIGNEVQKENFKEAFELLGAGILLEFVPELL
IPTILVFTIKSFIGSSENKNKIIKAINNSLMERETKWKEIYSWIVSNWLTRINTQFNKRK
EQMYQALQNQVDAIKTVIEYKYNNYTSDERNRLESEYNINNIREELNKKVSLAMENIERF
ITESSIFYLMKLINEAKVSKLREYDEGVKEYLLDYISEHRSILGNSVQELNDLVTSTLNN
SIPFELSSYTNDKILILYFNKLYKKIKDNSILDMRYENNKFIDISGYGSNISINGDVYIY
STNRNQFGIYSSKPSEVNIAQNNDIIYNGRYQNFSISFWVRIPKYFNKVNLNNEYTIIDC
IRNNNSGWKISLNYNKIIWTLQDTAGNNQKLVFNYTQMISISDYINKWIFVTITNNRLGN
SRIYINGNLIDEKSISNLGDIHVSDNILFKIVGCNDTRYVGIRYFKVFDTELGKTEIETL
YSDEPDPSILKDFWGNYLLYNKRYYLLNLLRTDKSITQNSNFLNINQQRGVYQKPNIFSN
TRLYTGVEVIIRKNGSTDISNTDNFVRKNDLAYINVVDRDVEYRLYADISIAKPEKIIKL
IRTSNSNNSLGQIIVMDSIGNNCTMNFQNNNGGNIGLLGFHSNNLVASSWYYNNIRKNTS
SNGCFWSFISKEHGWQEN
SEQ ID NO: 45-Polypeptide Sequence of BoNT/G-UniProt Q60393
MPVNIKXFNYNDPINNDDIIMMEPFNDPGPGTYYKAFRIIDRIWIVPERFTYGFQPDQFN
ASTGVFSKDVYEYYDPTYLKTDAEKDKFLKTMIKLFNRINSKPSGQRLLDMIVDAIPYLG
NASTPPDKFAANVANVSINKKIIQPGAEDQIKGLMTNLIIFGPGPVLSDNFTDSMIMNGH
SPISEGFGARMMIRFCPSCLNVFNNVQENKDTSIFSRRAYFADPALTLMHELIHVLHGLY
GIKISNLPITPNTKEFFMQHSDPVQAEELYTFGGHDPSVISPSTDMNIYNKALQNFQDIA
NRLNIVSSAQGSGIDISLYKQIYKNKYDFVEDPNGKYSVDKDKFDKLYKALMFGFTETNL
AGEYGIKTRYSYFSEYLPPIKTEKLLDNTIYTQNEGENIASKNLKTEFNGQNKAVNKEAY
EEISLEHLVIYRIAMCKPVMYKNTGKSEQCIIVNNEDLFFIANKDSFSKDLAKAETIAYN
TQNNTIENNFSIDQLILDNDLSSGIDLPNENTEPFTNFDDIDIPVYIKQSALKKIFVDGD
SLFEYLHAQTFPSNIENLQLTNSLNDALRNNNKVYTFFSTNLVEKANTVVGASLFVNWVK
GVIDDFTSESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLSTVNTQFYTI
KERMYNALNNQSQAIEKIIEDQYNRYSEEDKMNINIDFNDIDFKLNQSINLAINNIDDFI
NQCSISYLMNRMIPLAVKKLKDFDDNLKRDLLEYIDTNELYLLDEVNILKSKVNRHLKDS
IPFDLSLYTKDTILIQVFNNYISNISSNAILSLSYRGGRLIDSSGYGATMNVGSDVIFND
IGNGQFKLNNSENSNITAHQSKFVVYDSMFDNFSINFWVRTPKYNNNDIQTYLQNEYTII
SCIKNDSGWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRELNATEVS
SLYWIQSSTNTLKDFWGNPLRYDTQYYLFNQGMQNIYIKYFSKASMGETAPRTNFNNAAI
NYQNLYLGLRFIIKKASNSRNINNDNIVREGDYIYLNIDNISDESYRVYVLVNSKEIQTQ
LFLAPINDDPTFYDVLQIKKYYEKTTYNCQILCEKDTKTFGLFGIGKFVKDYGYVWDTYD
NYFCISQWYLRRISENINKLRLGCNWQFIPVDEGWTE
SEQ ID NO: 46-Polypeptide Sequence of TeNT-UniProt P04958
MPITINNFRYSDPVNNDTIIMMEPPYCKGLDIYYKAFKITDRIWIVPERYEFGTKPEDFN
PPSSLIEGASEYYDPNYLRTDSDKDRFLQTMVKLFNRIKNNVAGEALLDKIINAIPYLGN
SYSLLDKFDTNSNSVSFNLLEQDPSGATTKSAMLTNLIIFGPGPVLNKNEVRGIVLRVDN
KNYFPCRDGFGSIMQMAFCPEYVPTFDNVIENITSLTIGKSKYFQDPALLLMHELIHVLH
GLYGMQVSSHEIIPSKQEIYMQHTYPISAEELFTFGGQDANLISIDIKNDLYEKTLNDYK
AIANKLSQVTSCNDPNIDIDSYKQIYQQKYQFDKDSNGQYIVNEDKFQILYNSIMYGFTE
IELGKKFNIKTRLSYFSMNHDPVKIPNLLDDTIYNDTEGFNIESKDLKSEYKGQNMRVNT
NAFRNVDGSGLVSKLIGLCKKIIPPTNIRENLYNRTASLTDLGGELCIKIKNEDLTFIAE
KNSFSEEPFQDEIVSYNTKNKPLNFNYSLDKIIVDYNLQSKITLPNDRTTPVTKGIPYAP
EYKSNAASTIEIHNIDDNTIYQYLYAQKSPTTLQRITMTNSVDDALINSTKIYSYFPSVI
SKVNQGAQGILFLQWVRDIIDDFTNESSQKTTIDKISDVSTIVPYIGPALNIVKQGYEGN
FIGALETTGVVLLLEYIPEITLPVIAALSIAESSTQKEKIIKTIDNFLEKRYEKWIEVYK
LVKAKWLGTVNTQFQKRSYQMYRSLEYQVDAIKKIIDYEYKIYSGPDKEQIADEINNLKN
KLEEKANKAMININIFMRESSRSFLVNQMINEAKKQLLEFDTQSKNILMQYIKANSKFIG
ITELKKLESKINKVFSTPIPFSYSKNLDCWVDNEEDIDVILKKSTILNLDINNDIISDIS
GFNSSVITYPDAQLVPGINGKAIHLVNNESSEVIVHKAMDIEYNDMFNNFTVSFWLRVPK
VSASHLEQYGTNEYSIISSMKKHSLSIGSGWSVSLKGNNLIWTLKDSAGEVRQITFRDLP
DKFNAYLANKWVFITITNDRLSSANLYINGVLMGSAEITGLGAIREDNNITLKLDRCNNN
NQYVSIDKFRIFCKALNPKEIEKLYTSYLSITFLRDFWGNPLRYDTEYYLIPVASSSKDV
QLKNITDYMYLTNAPSYTNGKLNIYYRRLYNGLKFIIKRYTPNNEIDSFVKSGDFIKLYV
SYNNNEHIVGYPKDGNAFNNLDRILRVGYNAPGIPLYKKMEAVKLRDLKTYSVQLKLYDD
KNASLGLVGTHNGQIGNDPNRDILIASNWYFNHLKDKILGCDWYFVPTDEGWTND
SEQ ID NO: 47-Polypeptide Sequence of BoNT/X
MKLEINKFNYNDPIDGINVITMRPPRHSDKINKGKGPFKAFQVIKNIWIVPERYNFTNNT
NDLNIPSEPIMEADAIYNPNYLNTPSEKDEFLQGVIKVLERIKSKPEGEKLLELISSSIP
LPLVSNGALTLSDNETIAYQENNNIVSNLQANLVIYGPGPDIANNATYGLYSTPISNGEG
TLSEVSFSPFYLKPFDESYGNYRSLVNIVNKFVKREFAPDPASTLMHELVHVTHNLYGIS
NRNFYYNFDTGKIETSRQQNSLIFEELLTFGGIDSKAISSLIIKKIIETAKNNYTTLISE
RLNTVTVENDLLKYIKNKIPVQGRLGNFKLDTAEFEKKLNTILFVLNESNLAQRFSILVR
KHYLKERPIDPIYVNILDDNSYSTLEGFNISSQGSNDFQGQLLESSYFEKIESNALRAFI
KICPRNGLLYNAIYRNSKNYLNNIDLEDKKTTSKTNVSYPCSLLNGCIEVENKDLFLISN
KDSLNDINLSEEKIKPETTVFFKDKLPPQDITLSNYDFTEANSIPSISQQNILERNEELY
EPIRNSLFEIKTIYVDKLTTFHFLEAQNIDESIDSSKIRVELTDSVDEALSNPNKVYSPF
KNMSNTINSIETGITSTYIFYQWLRSIVKDFSDETGKIDVIDKSSDTLAIVPYIGPLLNI
GNDIRHGDFVGAIELAGITALLEYVPEFTIPILVGLEVIGGELAREQVEAIVNNALDKRD
QKWAEVYNITKAQWWGTIHLQINTRLAHTYKALSRQANAIKMNMEFQLANYKGNIDDKAK
IKNAISETEILLNKSVEQAMKNTEKFMIKLSNSYLTKEMIPKVQDNLKNFDLETKKTLDK
FIKEKEDILGTNLSSSLRRKVSIRLNKNIAFDINDIPFSEFDDLINQYKNEIEDYEVLNL
GAEDGKIKDLSGTTSDINIGSDIELADGRENKAIKIKGSENSTIKIAMNKYLRFSATDNF
SISFWIKHPKPTNLLNNGIEYTLVENFNQRGWKISIQDSKLIWYLRDHNNSIKIVTPDYI
AFNGWNLITITNNRSKGSIVYVNGSKIEEKDISSIWNTEVDDPIIFRLKNNRDTQAFTLL
DQFSIYRKELNQNEVVKLYNYYFNSNYIRDIWGNPLQYNKKYYLQTQDKPGKGLIREYWS
SFGYDYVILSDSKTITFPNNIRYGALYNGSKVLIKNSKKLDGLVRNKDFIQLEIDGYNMG
ISADRFNEDTNYIGTTYGTTHDLTTDFEIIQRQEKYRNYCQLKTPYNIFHKSGLMSTETS
KPTFHDYRDWVYSSAWYFQNYENLNLRKHTKTNWYFIPKDEGWDED
SEQ ID NO: 48-His-TEV Sequence
MHHHHHHDDDDK
SEQ ID NO: 49-Nucleotide Sequence Encoding mrBoNT/A
ATGCCATTCGTCAACAAGCAATTCAACTACAAAGACCCAGTCAACGGCGTCGACATCGC
ATACATCAAGATTCCGAACGCCGGTCAAATGCAGCCGGTTAAGGCTTTTAAGATCCACA
ACAAGATTTGGGTTATCCCGGAGCGTGACACCTTCACGAACCCGGAAGAAGGCGATCT
GAACCCGCCACCGGAAGCGAAGCAAGTCCCTGTCAGCTACTACGATTCGACGTACCTG
AGCACGGATAACGAAAAAGATAACTACCTGAAAGGTGTGACCAAGCTGTTCGAACGTAT
CTACAGCACGGATCTGGGTCGCATGCTGCTGACTAGCATTGTTCGCGGTATCCCGTTCT
GGGGTGGTAGCACGATTGACACCGAACTGAAGGTTATCGACACTAACTGCATTAACGTT
ATTCAACCGGATGGTAGCTATCGTAGCGAAGAGCTGAATCTGGTCATCATTGGCCCGAG
CGCAGACATTATCCAATTCGAGTGCAAGAGCTTTGGTCACGAGGTTCTGAATCTGACCC
GCAATGGCTATGGTAGCACCCAGTACATTCGTTTTTCGCCGGATTTTACCTTCGGCTTTG
AAGAGAGCCTGGAGGTTGATACCAATCCGTTGCTGGGTGCGGGCAAATTCGCTACCGA
TCCGGCTGTCACGCTGGCCCATGAACTGATCCACGCAGGCCACCGCCTGTACGGCATT
GCCATCAACCCAAACCGTGTGTTCAAGGTTAATACGAATGCATACTACGAGATGAGCGG
CCTGGAAGTCAGCTTCGAAGAACTGCGCACCTTCGGTGGCCATGACGCTAAATTCATTG
ACAGCTTGCAAGAGAATGAGTTCCGTCTGTACTACTATAACAAATTCAAAGACATTGCAA
GCACGTTGAACAAGGCCAAAAGCATCGTTGGTACTACCGCGTCGTTGCAGTATATGAAG
AATGTGTTTAAAGAGAAGTACCTGCTGTCCGAGGATACCTCCGGCAAGTTTAGCGTTGA
TAAGCTGAAGTTTGACAAACTGTACAAGATGCTGACCGAGATTTACACCGAGGACAACT
TTGTGAAATTCTTCAAAGTGTTGAATCGTAAAACCTATCTGAATTTTGACAAAGCGGTTTT
CAAGATTAACATCGTGCCGAAGGTGAACTACACCATCTATGACGGTTTTAACCTGCGTA
ACACCAACCTGGCGGCGAACTTTAACGGTCAGAATACGGAAATCAACAACATGAATTTC
ACGAAGTTGAAGAACTTCACGGGTCTGTTCGAGTTCTATAAGCTGCTGTGCGTGCGCGG
TATCATCACCAGCAAAACCAAAAGCCTGGACAAAGGCTACAACAAGGCGCTGAATGACC
TGTGCATTAAGGTAAACAATTGGGATCTGTTCTTTTCGCCATCCGAAGATAATTTTACCA
ACGACCTGAACAAGGGTGAAGAAATCACCAGCGATACGAATATTGAAGCAGCGGAAGA
GAATATCAGCCTGGATCTGATCCAGCAGTACTATCTGACCTTTAACTTCGACAATGAACC
GGAGAACATTAGCATTGAGAATCTGAGCAGCGACATTATCGGTCAGCTGGAACTGATGC
CGAATATCGAACGTTTCCCGAACGGCAAAAAGTACGAGCTGGACAAGTACACTATGTTC
CATTACCTGCGTGCACAGGAGTTTGAACACGGTAAAAGCCGTATCGCGCTGACCAACA
GCGTTAACGAGGCCCTGCTGAACCCGAGCCGTGTCTATACCTTCTTCAGCAGCGACTAT
GTTAAGAAAGTGAACAAAGCCACTGAGGCCGCGATGTTCCTGGGCTGGGTGGAACAGC
TGGTATATGACTTCACGGACGAGACGAGCGAAGTGAGCACTACCGACAAAATTGCTGAT
ATTACCATCATTATCCCGTATATTGGTCCGGCACTGAACATTGGCAACATGCTGTACAAA
GACGATTTTGTGGGTGCCCTGATCTTCTCCGGTGCCGTGATTCTGCTGGAGTTCATTCC
GGAGATTGCGATCCCGGTGTTGGGTACCTTCGCGCTGGTGTCCTACATCGCGAATAAG
GTTCTGACGGTTCAGACCATCGATAACGCGCTGTCGAAACGTAATGAAAAATGGGACGA
GGTTTACAAATACATTGTTACGAATTGGCTGGCGAAAGTCAATACCCAGATCGACCTGAT
CCGTAAGAAAATGAAAGAGGCGCTGGAGAATCAGGCGGAGGCCACCAAAGCAATTATC
AACTACCAATACAACCAGTACACGGAAGAAGAGAAGAATAACATTAACTTCAATATCGAT
GATTTGAGCAGCAAGCTGAATGAATCTATCAACAAAGCGATGATCAATATCAACAAGTTT
TTGAATCAGTGTAGCGTTTCGTACCTGATGAATAGCATGATTCCGTATGGCGTCAAACGT
CTGGAGGACTTCGACGCCAGCCTGAAAGATGCGTTGCTGAAATACATTTACGACAATCG
TGGTACGCTGATTGGCCAAGTTGACCGCTTGAAAGACAAAGTTAACAATACCCTGAGCA
CCGACATCCCATTTCAACTGAGCAAGTATGTTGATAATCAACGTCTGTTGAGCACTTTCA
CCGAGTATATCAAAAACATCATCAATACTAGCATTCTGAACCTGCGTTACGAGAGCAAGC
ATCTGATTGATCTGAGCCGTTATGCTAGCAAGATCAACATCGGTAGCAAGGTCAATTTTG
ACCCGATCGATAAGAACCAGATCCAGCTGTTTAATCTGGAATCGAGCAAAATTGAGGTT
ATCCTGAAAAAGGCCATTGTCTACAACTCCATGTACGAGAATTTCTCCACCAGCTTCTGG
ATTCGCATCCCGAAATACTTCAACAAGATTAGCCTGAACAACGAGTATACTATCATCAAC
TGTATGGAGAACAACAGCGGTTGGAAGGTGTCTCTGAACTATGGTGAGATCATTTGGAC
CTTGCAGGACACCAAAGAGATCAAGCAGCGCGTCGTGTTCAAGTACTCTCAAATGATCA
ACATTTCCGATTACATTAATCGTTGGATCTTCGTGACCATTACGAATAACCGTCTGAATA
AGAGCAAGATTTACATCAATGGTCGCTTGATCGATCAGAAACCGATTAGCAACCTGGGT
AATATCCACGCAAGCAACAAGATTATGTTCAAATTGGACGGTTGCCGCGATACCCATCG
TTATATCTGGATCAAGTATTTCAACCTGTTTGATAAAGAACTGAATGAGAAGGAGATCAA
AGATTTGTATGACAACCAATCTAACAGCGGCATTTTGAAGGACTTCTGGGGCGATTATCT
GCAATACGATAAGCCGTACTATATGCTGAACCTGTATGATCCGAACAAATATGTGGATGT
CAATAATGTGGGTATTCGTGGTTACATGTATTTGAAGGGTCCGCGTGGCAGCGTTATGA
CGACCAACATTTACCTGAACTCTAGCCTGTACCGTGGTACGAAATTCATCATTAAGAAAT
ATGCCAGCGGCAACAAAGATAACATTGTGCGTAATAACGATCGTGTCTACATCAACGTG
GTCGTGAAGAATAAAGAGTACCGTCTGGCGACCAACGCTTCGCAGGCGGGTGTTGAGA
AAATTCTGAGCGCGTTGGAGATCCCTGATGTCGGTAATCTGAGCCAAGTCGTGGTTATG
AAGAGCAAGAACGACAAGGGTATCACTAACAAGTGCAAGATGAACCTGCAAGACAACAA
TGGTAACGACATCGGCTTTATTGGTTTCCACCAGTTCAACAATATTGCTAAACTGGTAGC
GAGCAATTGGTACAATCGTCAGATTGAGCGCAGCAGCcGTACTTTGGGCTGTAGCTGGG
AGTTTATCCCGGTCGATGATGGTTGGGGCGAACGTCCGCTG
SEQ ID NO: 50-Nucleotide Sequence Encoding an Extracellular Portion (Amino Acids
1-61) of Human Modified SYTII (L51F)
ATGCGTAACATCTTCAAACGTAACCAAGAGCCGATTGTTGCGCCGGCGACCACCACCG
CGACCATGCCGATTGGTCCGGTTGACAACAGCACCGAAAGCGGTGGCGCGGGTGAAA
GCCAAGAAGATATGTTTGCGAAGCTGAAAGAGAAGTTCTTTAACGAAATCAACAAGATTC
CGCTGCCG
SEQ ID NO: 51-Nucleotide Sequence Encoding an SV2c Capture Substrate
atgggctggagctgcattattctgtttctggtggcgaccgcgaccggcgtgcatagcggc
ggcggcggcagcagcccgattctgggctattggaaaattaaaggcctggtgcagccgacc
cgcctgctgctggaatatctggaagaaaaatatgaagaacatctgtatgaacgcgatgaa
ggcgataaatggcgcaacaaaaaatttgaactgggcctggaatttccgaacctgccgtat
tatattgatggcgatgtgaaactgacccagagcatggcgattattcgctatattgcggat
aaacataacatgctgggcggctgcccgaaagaacgcgcggaaattagcatgctggaaggc
gcggtgctggatattcgctatggcgtgagccgcattgcgtatagcaaagattttgaaacc
ctgaaagtggattttctgagcaaactgccggaaatgctgaaaatgtttgaagatcgcctg
tgccataaaacctatctgaacggcgatcatgtgacccatccggattttatgctgtatgat
gcgctggatgtggtgctgtatatggatccgatgtgcctggatgcgtttccgaaactggtg
tgctttaaaaaacgcattgaagcgattccgcagattgataaatatctgaaaagcagcaaa
tatattgcgtggccgctgcagggctggcaggcgacctttggcggcggcgatcatccgccg
aaaagcgatctgagcagcggcctggaagtgctgtttcagggcccggtggaacgcgataaa
tatgcgaactttaccattaactttaccatggaaaaccagattcataccggcatggaatat
gataacggccgctttattggcgtgaaatttaaaagcgtgacctttaaagatagcgtgttt
aaaagctgcacctttgaagatgtgaccagcgtgaacacctattttaaaaactgcaccttt
attgataccgtgtttgataacaccgattttgaaccgtataaatttattgatagcgaattt
aaaaactgcagcttttttcataacaaaacc
SEQ ID NO: 75-C-terminal L-chain Fragment
TKSLDKGYNK
SEQ ID NO: 76-C-terminal L-chain Fragment 2
SLDKGYNK
SEQ ID NO: 77-Di-Chain L-Chain 1
PFVNKQFNYKDPVNGVDIAYIKIPNAGQMQPVKAFKIHNKIWVIPERDTFTNPEEGDLNPPPE
AKQVPVSYYDSTYLSTDNEKDNYLKGVTKLFERIYSTDLGRMLLTSIVRGIPFWGGSTIDTEL
KVIDTNCINVIQPDGSYRSEELNLVIIGPSADIIQFECKSFGHEVLNLTRNGYGSTQYIRFSPDF
TFGFEESLEVDTNPLLGAGKFATDPAVTLAHELIHAGHRLYGIAINPNRVFKVNTNAYYEMSG
LEVSFEELRTFGGHDAKFIDSLQENEFRLYYYNKFKDIASTLNKAKSIVGTTASLQYMKNVFK
EKYLLSEDTSGKFSVDKLKFDKLYKMLTEIYTEDNFVKFFKVLNRKTYLNFDKAVFKINIVPKV
NYTIYDGFNLRNTNLAANFNGQNTEINNMNFTKLKNFTGLFEFYKLLCVRGIITSK
SEQ ID NO: 78-Di-Chain L-Chain 2
PFVNKQFNYKDPVNGVDIAYIKIPNAGQMQPVKAFKIHNKIWVIPERDTFTNPEEGDLNPPPE
AKQVPVSYYDSTYLSTDNEKDNYLKGVTKLFERIYSTDLGRMLLTSIVRGIPFWGGSTIDTEL
KVIDTNCINVIQPDGSYRSEELNLVIIGPSADIIQFECKSFGHEVLNLTRNGYGSTQYIRFSPDF
TFGFEESLEVDTNPLLGAGKFATDPAVTLAHELIHAGHRLYGIAINPNRVFKVNTNAYYEMSG
LEVSFEELRTFGGHDAKFIDSLQENEFRLYYYNKFKDIASTLNKAKSIVGTTASLQYMKNVFK
EKYLLSEDTSGKFSVDKLKFDKLYKMLTEIYTEDNFVKFFKVLNRKTYLNFDKAVFKINIVPKV
NYTIYDGFNLRNTNLAANFNGQNTEINNMNFTKLKNFTGLFEFYKLLCVRGIITSKTK
SEQ ID NO: 79-Di-Chain H-Chain
ALNDLCIKVNNWDLFFSPSEDNFTNDLNKGEEITSDTNIEAAEENISLDLIQQYYLTFNFDNEP
ENISIENLSSDIIGQLELMPNIERFPNGKKYELDKYTMFHYLRAQEFEHGKSRIALTNSVNEAL
LNPSRVYTFFSSDYVKKVNKATEAAMFLGWVEQLVYDFTDETSEVSTTDKIADITIIIPYIGPAL
NIGNMLYKDDFVGALIFSGAVILLEFIPEIAIPVLGTFALVSYIANKVLTVQTIDNALSKRNEKWD
EVYKYIVTNWLAKVNTQIDLIRKKMKEALENQAEATKAIINYQYNQYTEEEKNNINFNIDDLSS
KLNESINKAMININKFLNQCSVSYLMNSMIPYGVKRLEDFDASLKDALLKYIYDNRGTLIGQVD
RLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYT
IINCMKNNSGWKISIRGNRIIWTLIDINGKTKSVFFEYNIREDISEYINRWFFVTITNNLNNAKIYI
NGKLESNTDIKDIREVIANGEIIFKLDGDIDRTQFIWMKYFSIFNTELSQSNIEERYKIQSYSEYL
KDFWGNPLMYNKEYYMFNAGNKNSYIKLKKDSPVGEILTRSKYNQNSKYINYRDLYIGEKFII
RRKSNSQSINDDIVRKEDYIYLDFFNLNQEWRVYTYKYFKKEEMKLFLAPIYDSDEFYNTIQIK
EYDEQPTYSCQLLFKKDEESTDEIGLIGIHRFYESGIVFEEYKDYFCISKWYLKEVKRKPYNL
KLGCNWQFIPKDEGWTE
SEQ ID NO: 80-Polypeptide Sequence of an Extracellular Portion (Amino Acids 1-60)
of Human SYT-I
MVSESHHEALAAPPVTTVATVLPSNATEPASPGEGKEDAFSKLKEKFMNELHKIPLPPWA
SEQ ID NO: 81-Polypeptide Sequence of Capture Substrate Comprising an
Extracellular Portion (Amino Acids 1-60) of Human SYT-I
MSGSHHHHHHSSGMSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMCLDAF
PKLVCFKKRIEAIPQIDKYLKSSKYIAWPLQGWQATFGGGDHPPKSDLGHTGHRSGTENLYF
QGMVSESHHEALAAPPVTTVATVLPSNATEPASPGEGKEDAFSKLKEKFMNELHKIPLPPW
A
SEQ ID NO: 82-Polypeptide Sequence of Full-Length Mouse SYT-I
MVSASRPEALAAPVTTVATLVPHNATEPASPGEGKEDAFSKLKQKFMNELHKIPLPPWALIAI
AIVAVLLVVTCCFCVCKKCLFKKKNKKKGKEKGGKNAINMKDVKDLGKTMKDQALKDDDAE
TGLTDGEEKEEPKEEEKLGKLQYSLDYDFQNNQLLVGIIQAAELPALDMGGTSDPYVKVFLL
PDKKKKFETKVHRKTLNPVFNEQFTFKVPYSELGGKTLVMAVYDFDRFSKHDIIGEFKVPMN
TVDFGHVTEEWRDLQSAEKEEQEKLGDICFSLRYVPTAGKLTVVILEAKNLKKMDVGGLSD
PYVKIHLMQNGKRLKKKKTTIKKNTLNPYYNESFSFEVPFEQIQKVQVVVTVLDYDKIGKNDA
IGKVFVGYNSTGAELRHWSDMLANPRRPIAQWHTLQVEEEVDAMLAVKK
SEQ ID NO: 83-Polypeptide Sequence of an Extracellular Portion (Amino Acids 1-59)
of Mouse SYT-I
MVSASRPEALAAPVTTVATLVPHNATEPASPGEGKEDAFSKLKQKFMNELHKIPLPPWA
SEQ ID NO: 84-Polypeptide Sequence of Capture Substrate Comprising an
Extracellular Portion (Amino Acids 1-59) of Mouse SYT-I
MSGSHHHHHHSSGMSPILGYWKIKGLVQPTRLLLEYLEEKYEEHLYERDEGDKWRNKKFEL
GLEFPNLPYYIDGDVKLTQSMAIIRYIADKHNMLGGCPKERAEISMLEGAVLDIRYGVSRIAYS
KDFETLKVDFLSKLPEMLKMFEDRLCHKTYLNGDHVTHPDFMLYDALDVVLYMDPMCLDAF
PKLVCFKKRIEAIPQIDKYLKSSKYIAWPLQGWQATFGGGDHPPKSDLGHTGHRSGTENLYF
QGMVSASRPEALAAPVTTVATLVPHNATEPASPGEGKEDAFSKLKQKFMNELHKIPLPPWA

### EXAMPLES

### Example 1

### Materials & Methods

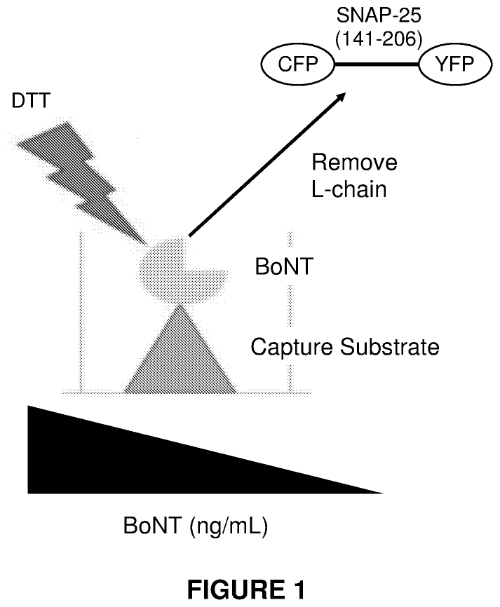

Relevant capture substrates for the neurotoxin serotype were immobilised onto clear maxisorp 96 well microtitre plates at 5 ug/mL in Dulbecco's PBS for 1 hour at 37° C. shaking at 600 rpm before blocking with 150 μL 1% BSA Dulbecco's PBS 0.05% Tween20. Serial dilutions of relevant neurotoxin test samples were then applied to the plate in 1% BSA Dulbecco's PBS 0.05% Tween20. After 1 hour shaking at 600 rpm at 37° C., unbound neurotoxin was then removed from the plate via washing 1× in 1% BSA Dulbecco's PBS 0.05% Tween20. Bound toxin was then liberated from the capture substrates via reduction in 50 mM Hepes-NaOH, pH 7.1, 5 mM NaCl, 0.1% Tween-20, 10 μM ZnCl, 5 mM DTT for 15 minutes shaking at 600 rpm at 37° C. The supernatant was then transferred to a black opaque microtitre plate containing 2 μM CFP/YFP flanked SNAP25 substrate (see FIG. 1), before incubation at 37° C. at 600 rpm for 3 hours, followed by measuring fluorescence at an excitation/emission of 434/526 (FRET signal), and 434/470 (CFP signal).

### Results

A novel plate based assay platform for accessing clostridial neurotoxin affinity to its receptor, as well as its endopeptidase activity was developed (FIG. 1). This assay utilised the commercially available BoTest endopeptidase reporter system (BioSentinel), combined with a bespoke recombinantly expressed receptor substrate (capture substrate), to measure toxin light-chain and heavy-chain quality respectively. The aim of the method was to: (i) provide accurate sample potency prediction to limit cell-based assay demand; and (ii) characterise and understand the mechanism of action during toxin sample degradation.

### Substrates

For the binding of mrBoNT/A (SEQ ID NO: 14 converted into a di-chain toxin by incubation with Lys-C—see WO2014/080206, which is incorporated herein by reference), the toxin binding region comprising SV2c (519-563) was employed. Recombinant GST-SV2c (amino acids 473-567) shown as SEQ ID NO: 29 was expressed in E. coli.

In order to generate similar material with eukaryotic peptide glycosylations, the GST-SV2c (473-567) substrate (SEQ ID NO: 40) was also generated in a HEK293 mammalian cells. Mass spectrometry analysis was performed and indicated a mixture of the glycans GOF, G1F and G2F suggesting consistent, but heterogenous glycosylation of the GST-SV2c (473-567) substrate when expressed in mammalian HEK293 cells (see table below).

| | N-linked oligos observed | |
|---|---|---|
| | Batch 2 | Batch 3 |
| Site | (trypsin digest) | (trypsin digest) |
| T40-41: | G2f, G1f, G0f | Man-5, G0f-GlcNAc, |
| FIDSEFKNCSFFHNK | | G0f, G1f, G2f |
| T41: NCSFFHNK | G2f, G1f, G0f and | Man-5 |
| | aglycosylated | |
| T41-42: NCSFFHNKT | Aglycosylated | Man-5 |

In particular, glycosylation was observed at physiologically-relevant amino acid residue N559.

For the binding of mrBoNT/AB (SEQ ID NO: 7 converted into a di-chain toxin by incubation with Lys-C), synaptotagmin II (SYTII) substrate was produced in E. coli, GST tagged SYTII (1-61). The SYTII was human with an L51F amino acid mutation (SEQ ID NO: 20).

### Characterisation of Capture Substrate Binding

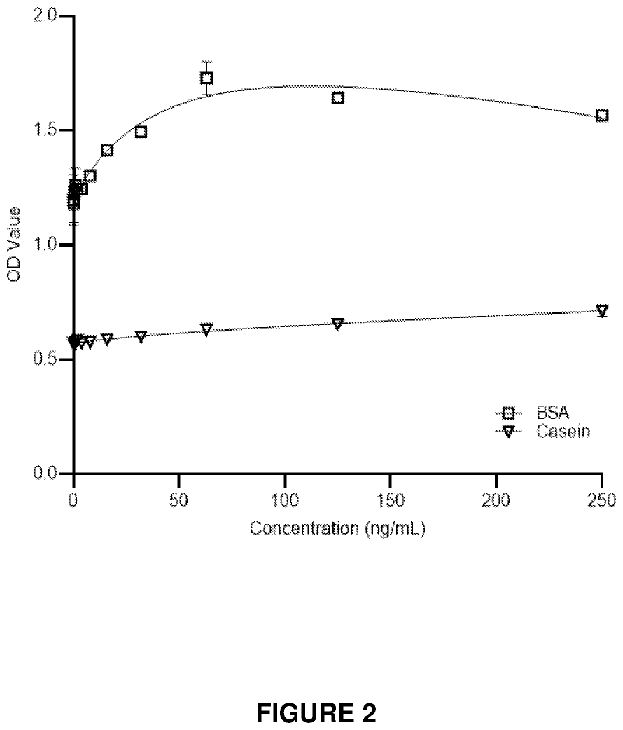

Following toxin serial dilution and application to the plate, the captured toxin was probed with an anti-BoNT antibody before secondary detection with a horseradish peroxidase (HRP) coupled anti-species antibody. The level of binding to the capture receptor was proportionate to the neurotoxin's affinity to the capture substrates. The effect of carrier protein blocking buffers on assay sensitivity was assessed. Surprisingly, when compared to casein, using BSA (1%) as a blocker improved binding (FIG. 2).

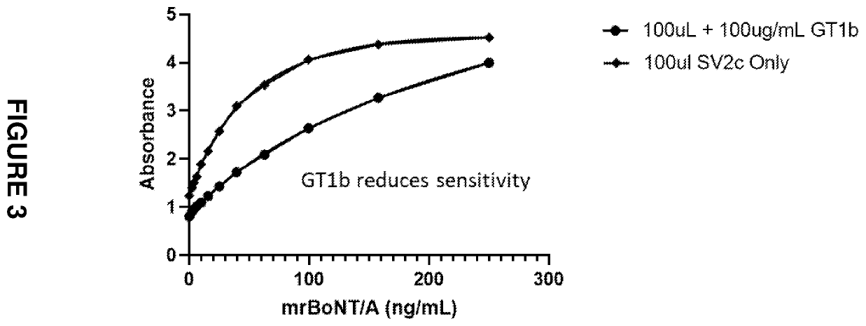

In order to increase toxin binding, several attempts at co-immobilising the ganglioside GT1b to plates along with capture substrates comprising an extracellular portion of SV2c were made. Neither pre-coating before SV2c portion capture substrate immobilisation, nor co-immobilisation of GT1b with SV2c portion capture substrate improved toxin capture over SV2c portion capture substrate alone. In fact, surprisingly, co-immobilisation using GT1b actually reduced sensitivity of the assay (FIG. 3). Therefore, to improve sensitivity, it was considered advantageous to exclude the use of a ganglioside (e.g. GT1b) in the binding and cleavage assay.

### Binding & Cleavage Assay

Having characterised binding, the full capture substrate binding and cleavage assay was characterised. The assay was carried out as described above. Briefly, capture substrates were immobilised in wells of multi-well plates, prior to incubation with mrBoNT/A or mrBoNT/AB respectively. The BoNT-capture substrate complexes were washed and a buffer containing 50 mM HEPES-NaOH, PH 7.1, 5 mM NaCl, 0.1% Tween-20, 10 μM ZnCl₂, and 5 mM DTT was added for liberation of the light-chain from the bound toxin. The supernatant was then transferred to an additional assay plate containing the CFP/YFP FRET-based cleavable substrates (FIG. 1). This allowed for measuring the light-chain endopeptidase activity of toxin that has specifically bound to its respective capture substrate, which removes the contribution of free light-chain polypeptides that may be produced through toxin degradation and impurities.

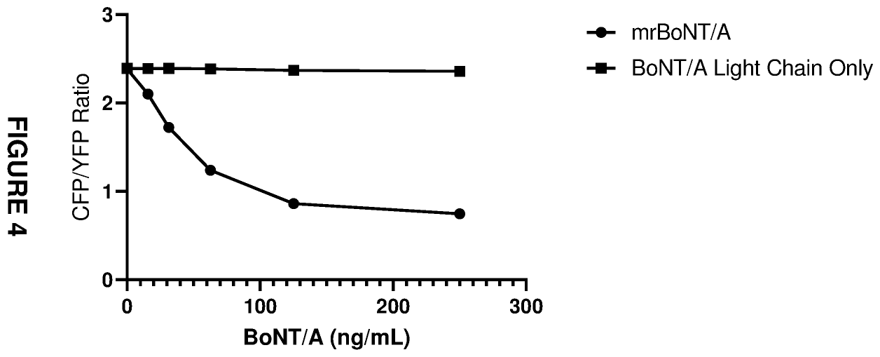

In order to assess the contribution of any non-specific endopeptidase activity carry over from the toxin binding step, a recombinant light-chain only control was used. FIG. 4 shows that no endopeptidase activity was detected in samples containing only recombinant light-chain showing that there is no carry over of neurotoxin that has not bound to its specific receptor. Thus, the assay was shown to have little/no non-specific background.

### Assay Qualification

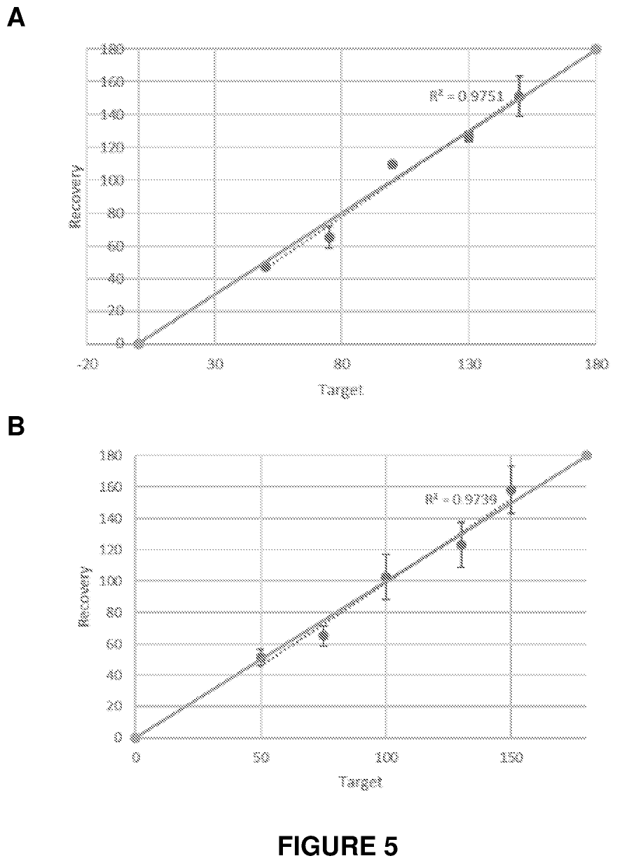

In order to assess accuracy, precision and reproducibility, the binding and cleavage assay was performed in triplicate over 5 levels from 50% to 150% for mrBoNT/A and mrBoNT/AB, respectively. As shown in FIG. 5A, the assay shows a high degree of accuracy and precision over 5 levels of the assay. Standard deviation of triplicates over 5 levels is on average less than 6%, and the average recovery to target is less than 5% drift. This therefore demonstrates that the assay has good linearity, accuracy and precession between 50% and 150%. As shown in FIG. 5B, the assay composition also shows comparable accuracy, linearity and precision when carried out using a composition comprising mrBoNT/AB. The table below presents a summary for assays carried out with mrBoNT/A or mrBoNT/AB compositions.

| | mrBoNT/A | mrBoNT/AB |
|---|---|---|
| Average triplicate standard deviation | 5.0 | 14.4 |
| Average recovery to target drift+/− | 1.0 | 0.84 |

### Determination of Clostridial Neurotoxin Composition Stability

In order to test the stability indicating, comparability, and potency-predicting properties of the assay, degraded samples of mrBoNT/A and mrBoNT/A were tested and compared to orthogonal binding methods, as well as mass spectroscopy and cell based methods.

Oxidised mrBoNT/A

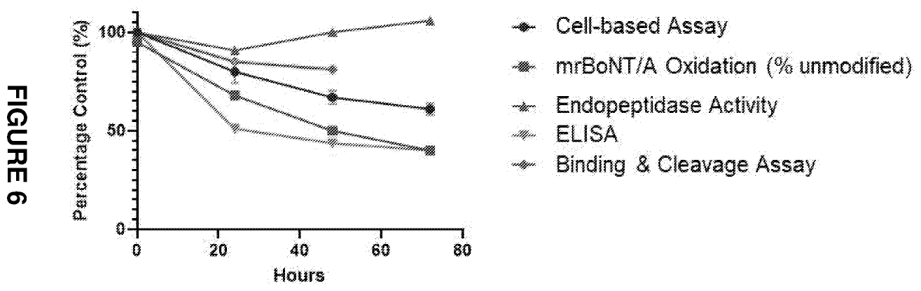

FIG. 6 shows that the binding and cleavage assay showed high comparability to existing cell-based methods and the activity level determined correlated with the amount of oxidised mrBoNT/A present in the composition. mrBoNT/A forced oxidation did not appear to effect light-chain activity alone when tested with the standard BoTest, however when tested in the binding and cleavage assay, potency estimation closely aligned with total physiological potency as tested by cell-based assay.

mrBoNT/A samples from the same oxidation study were also assessed via bi-layer interferometry (BLI) using the Octet Red 96e system, both with glycosylated and non-glycosylated capture substrates comprising a GST-tagged extracellular portion of SV2c.

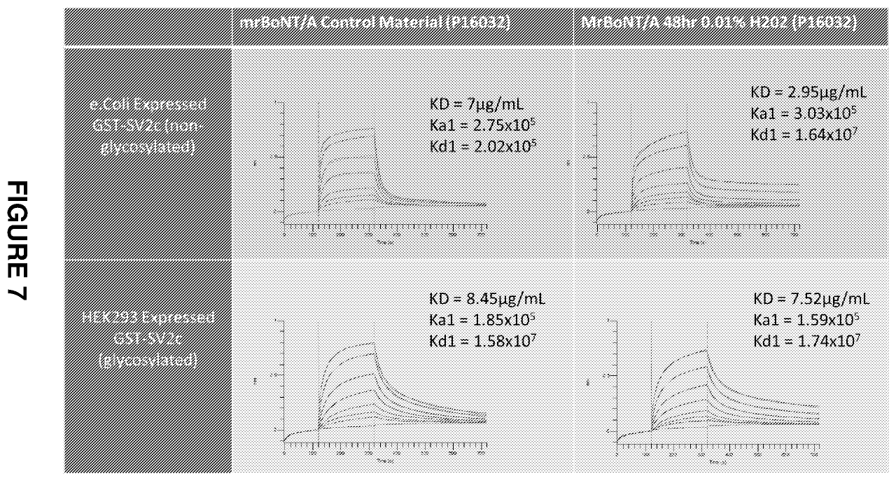

As shown in FIG. 7, the non-glycosylated capture substrates bound to mrBoNT/A faster than the glycosylated form. However the glycosylated capture substrates had a significantly slower dissociation rate from the toxin. Oxidised mrBoNT/A shows a reduction in capture substrate binding for both glycosylated and non-glycosylated forms. The dissociation rate of the toxin is also prolonged with oxidised mrBoNT/A with both types of (glycosylated and non-glycosylated) capture substrate.

Thus, taking the results of FIGS. 6 and 7 together, the binding and cleavage assay was able to detect differences in activity of oxidised BoNTs, which is likely as a result of decreased capture substrate binding.

Oxidised mrBoNT/AB

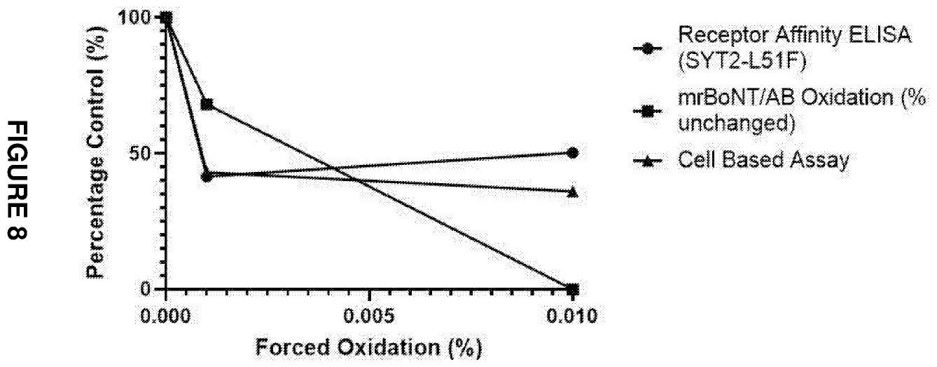

Next, forced oxidised samples of mrBoNT/AB were analysed via ELISA as described above with capture substrates comprising an extracellular portion of an L51F mutated human SYTII. FIG. 8 shows that oxidation of the H_{C} domain of mrBoNT/AB was associated with a reduction in activity as assessed via a cell-based assay and reduced receptor binding via ELISA.

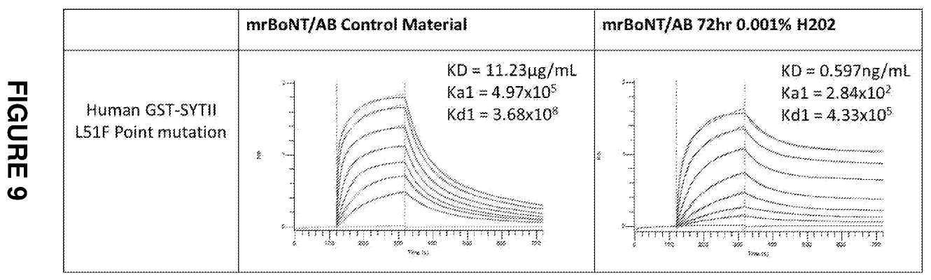

Similarly, to mrBoNT/A, forced oxidation samples were tandem tested with BLI (FIG. 9). The binding affinity of oxidised mrBoNT/AB to capture substrates comprising the L51F mutant SYTII was reduced.

Hence, these results show that oxidised mrBoNT/AB has a different receptor binding profile than equivalent non-oxidised mrBoNT/AB. Thus, it is plausible that the binding and cleavage assay will allow a distinction to made between the activity level of oxidised versus non-oxidised mrBoNT/AB. Moreover, it is plausible that the binding and cleavage assay can be used to attribute loss of activity to a specific part of the toxin's mechanism of action (e.g. binding and/or proteolytic activity), thereby providing mechanistic insights into the root cause of loss of activity of a composition.

### Correlation to Cell-Based Activity Results

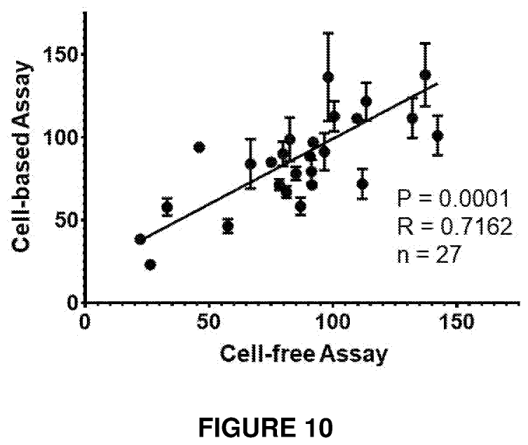

As the binding and cleavage assay measures both the heavy-chain affinity of a toxin composition, and the endopeptidase activity of the toxin, which has successfully bound to its respective receptor, samples which produce a signal in this assay are highly likely to be physiologically active. To confirm this, the binding and cleavage assay was used to test a given composition and the same composition was, in parallel, tested using the cell-based assay. FIG. 10 shows that there is a highly significant correlation between activity data obtained using the binding and cleavage assay and a cell-based assay (P=0.0001). This dataset comprised 27 samples, which included samples mrBoNT/AB or mrBoNT/A samples subjected to forced degradation (including oxidation, glycation, and thermal stress) or samples obtained from various points in the mrBoNT/AB production process (including those of varying purity levels, or expressed or isolated under different conditions).

### Conclusions

In conclusion, the binding and cleavage assay reliably predicts physiological activity of a clostridial neurotoxin composition. As the data correlated strongly with cell-based assay results, the binding and cleavage assay may also be used as a high-throughput, screening tool to triage large numbers of compositions before cell-based testing. Through testing of samples, the assay has demonstrated stability indication for several toxin covalent post-translational modifications such as oxidation and glycation, which can be attributed to specific domains. The assay has also demonstrated stability indication of toxin manufacture process changes with unknown structural/biological differences. The results in this Example suggested that loss of neurotoxin potency can be predicted and specifically attributed to domains of toxins samples by a combined approach (see Example 3).

### Example 2

### Materials & Methods

Relevant capture substrates for the neurotoxin serotype were immobilised onto white maxisorp 96 well microtitre plates at 5 ug/mL in Dulbecco's PBS for 1 hour at 37° C. shaking at 600 rpm before blocking with 150 μL 1% BSA Dulbecco's PBS 0.05% Tween20. Serial dilutions of relevant neurotoxin test samples were then applied to the plate in 1% BSA Dulbecco's PBS 0.05% Tween20. After 1 hour shaking at 600 rpm at 37° C., unbound neurotoxin was then removed from the plate via washing 1× in 1% BSA Dulbecco's PBS 0.05% Tween20. Bound toxin was then liberated from the capture substrates via reduction in 50 mM Hepes-NaOH, pH 7.1, 0.1 mg/mL BSA, 0.1% Tween-20, 50 μM ZnCl, 5 mM DTT with 0.1 μM cleavage substrate comprising a first luciferase domain, second luciferase domain and SNAP-25 cleavage site (SEQ ID NO: 6 plus N-terminal His-TEV tag [SEQ ID NO: 48]). The assay plate was then shaken at 600 rpm at 37° C. for 3 hours. 50 μL of Furimazine at 37.5 μM diluted in 50 mM HEPES-NaOH, 0.1% Tween20, 50 μM ZnCl, 5 mM DTT was then added to all wells. Luminescence was subsequently captured using a plate reader based method.

### Results

### Cleavable Substrates

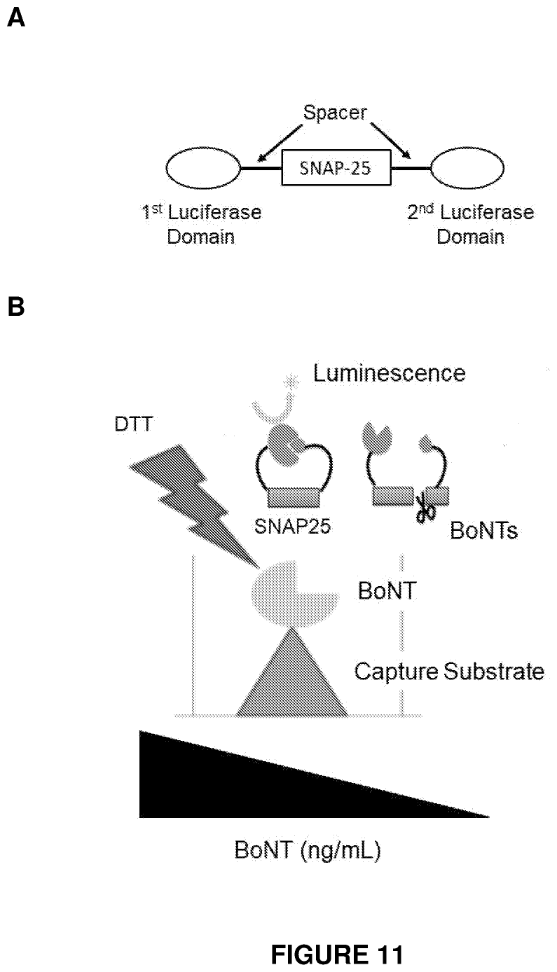

A new cleavable substrate (comprising SEQ ID NO: 6) was produced comprising an N-terminal first luciferase domain (SEQ ID NO: 2) and a C-terminal second luciferase domain (SEQ ID NO: 3) flanking a linker comprising a SNAP-25 cleavage site flanked by two spacers (FIG. 11A).

### Modified Binding & Cleavage Assay

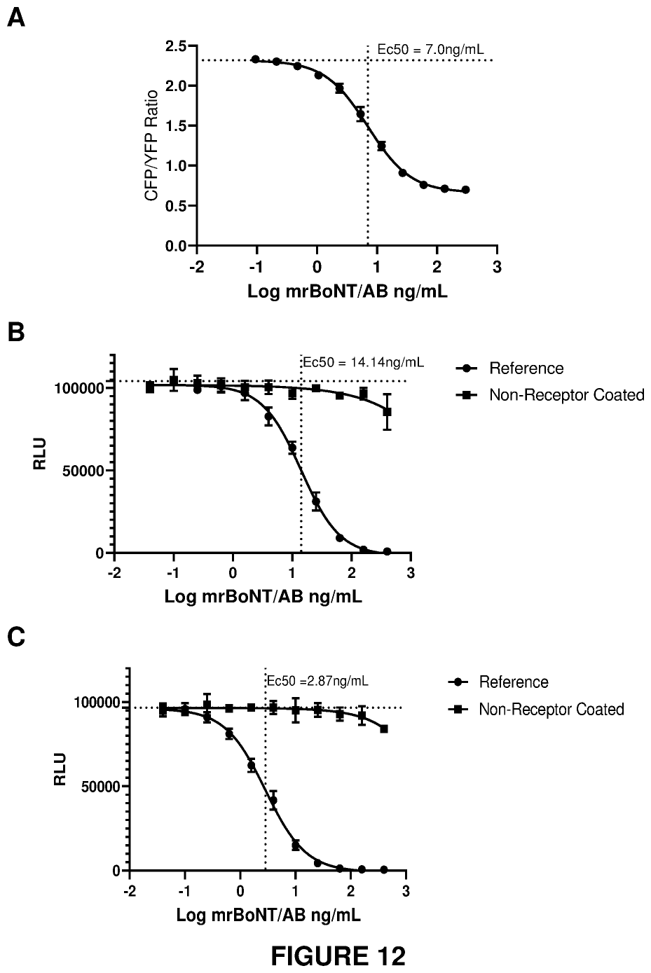

The binding and cleavage assay was carried out according to Example 1 but substituting the CFP/YFP cleavage substrates with that shown in FIG. 11A (and measuring luminescence in a white maxisorp 96 well microtitre plate). However, it was found that the sensitivity of the assay was much lower (FIG. 12B) when compared to the same assay, with the same mrBoNT/AB composition employing the CFP/YFP cleavage substrates (FIG. 12A).

As part of the assay characterisation procedure, the assay was carried out as shown in FIG. 11B and as described in the Example 2 Materials & Methods section. Specifically, together with the addition of DTT, the luciferase-based cleavage substrates were added to the well (containing the clostridial neurotoxin-capture substrate complexes) and incubated.

Subsequently, luciferase substrate (furimazine [Carbosynth ZEC04024]) was added, and luminescence assessed.

Prior to carrying out the experiment, it was expected that the non-specific background binding would have been substantial and that specificity of the assay would have been negatively affected. Unexpectedly, this was not the case and it was found that adopting this method not only significantly improved the sensitivity of the assay (FIG. 12C), but did so without increasing non-specific background, which was minimal as shown by a control assay in which capture substrates had not been immobilised on the assay plate (FIG. 12C, square). In fact, the sensitivity was substantially improved when compared to the assay of Example 1 employing the CFP/YFP cleavage substrates (FIG. 12A).

In view of this finding, advantageously, the number of plates used for a given assay can be reduced. This means that the modified binding and cleavage assay is associated with reduced wastage and cost, and is more amenable to high-throughput testing.

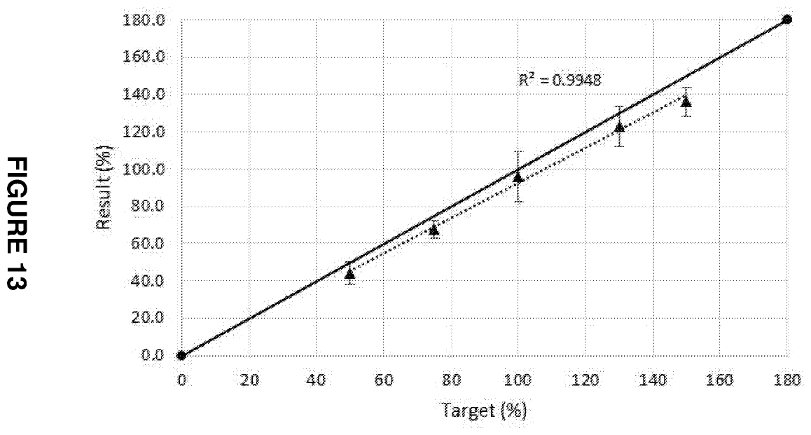

The modified binding and cleavage assay was performed in triplicate over 5 levels from 50% to 150% for mrBoNT/AB to further interrogate assay accuracy, precision and reproducibility. As shown in FIG. 13, the assay is associated with a high degree of accuracy and precision over 5 levels. Standard deviation of triplicates over 5 levels is on average 8.6%, and the average recovery to target is less than 0.4% drift. This therefore demonstrates that the assay has good linearity, accuracy and precession between 50% and 150%.

### Characterisation of Clostridial Neurotoxin Formulations

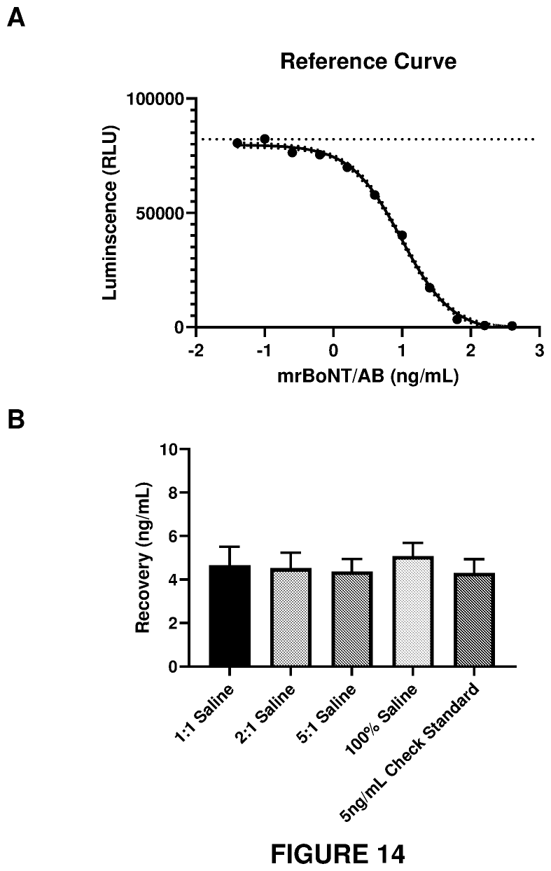

FIG. 14A shows a reference curve for an mrBoNT/AB composition produced using an assay according to Example 1, but employing cleavable substrates shown in FIG. 11A (comprising first and second luciferase domains and measuring luminescence in a white maxisorp 96 well microtitre plate). When assessing different cosmetic or therapeutic clostridial neurotoxin formulations, assay interference from various excipients and saline based reconstitution buffers may occur. The assay according to Example 1 lacked the required sensitivity to achieve a parallel linear potency assay.

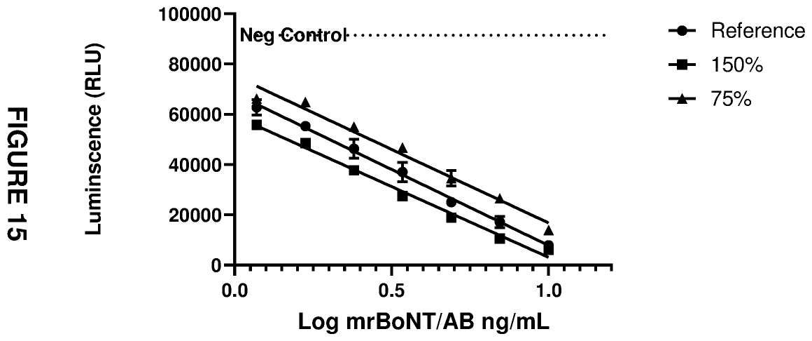

In contrast, owing to an associated improved sensitivity, the modified binding and cleavage assay as shown in FIG. 11B demonstrated linearity such that very small differences in activity could be detected (FIG. 15). As shown in FIG. 14B, the modified binding and cleavage assay was not significantly affected by increasing saline recon which alters excipient and buffer concentrations. Thus, the modified binding and cleavage assay is particularly suited to characterising/screening appropriate excipients and/or excipient concentrations for production for formulating therapeutic and/or cosmetic clostridial neurotoxin compositions.

### Example 3

### Materials & Methods

### Heavy-Chain Receptor Affinity ELISA

Relevant capture substrates for the neurotoxin serotype were immobilised onto clear maxisorp 96 well microtitre plates at 5 ug/mL in Dulbecco's PBS for 1 hour at 37° C. shaking at 600 rpm before blocking with 150 μL 1% BSA Dulbecco's PBS 0.05% Tween20. Serial dilutions of relevant neurotoxin test samples were then applied to the plate in 1% BSA Dulbecco's PBS 0.05% Tween20. After 1 hour shaking at 600 rpm at 37° C., unbound neurotoxin was then removed from the plate via washing 1× in 1% BSA Dulbecco's PBS 0.05% Tween20. Captured toxin was then fixed in 2% paraformaldehyde at room temperature for 10 minutes, before an additional wash in 1% BSA Dulbecco's PBS 0.05% Tween20. Anti-BoNT antibody diluted in 1% BSA Dulbecco's PBS 0.05% Tween20 was then applied at 2 μg/mL, followed by a 1 hour 37° C. incubation with shaking at 600 rpm. Plates were then washed once in 1% BSA Dulbecco's PBS 0.05% Tween20 before the addition of the relevant anti-species HRP conjugated antibody at 0.4 μg/mL. Following a final incubation at 37° C. for 1 hour with shaking at 600 rpm, plates were then washed twice with Dulbecco's PBS, before addition of pre-warmed TMB (3,3′,5,5′-Tetramethylbenzidine) substrate (ThermoFisher N301) for 5 minutes, before being quenched with ELISA stop solution (0.16M sulfuric acid, TMB Stop solution, ThermoFisher N600).

### Endopeptidase Activity Only

Relevant BoNT neurotoxin preparations were serially diluted in 50 mM HEPES NaOH, 50 μM ZnCl 0.1% Tween20, 5 mM DTT. Prepared serial dilutions of neurotoxin were then added to white 96 well microtitre plates pre-loaded with 10 μL of 1 μM cleavage substrate comprising a first luciferase domain, second luciferase domain and SNAP-25 cleavage site (comprising SEQ ID NO: 6) diluted in 50 mM HEPES NaOH, 50 μM ZnCl 0.1% Tween20, 5 mM DTT. Plates were then incubated for 3 hours at 37° C. shaking at 600 rpm. 50 μL of 37.5 μM furimazine diluted in 50 mM HEPES NaOH, 50 μM ZnCl 0.1% Tween20, 5 mM DTT was then added to all wells. Luminescence was then captured using a plate reader based method.

### Results

Samples of mrBoNT/AB and mrBoNT/A (respectively) were subjected to forced degradation and tested by way of a heavy-chain receptor affinity ELISA, in vitro endopeptidase assay; or the binding and cleavage assay described herein.

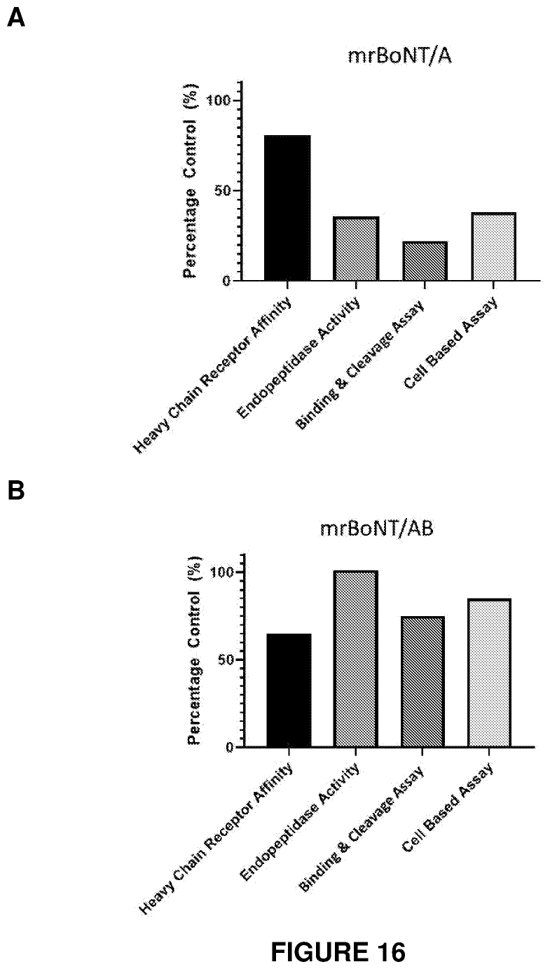

FIG. 16 shows that combining: (i) a heavy-chain receptor affinity ELISA; (ii) an in vitro endopeptidase assay; and (iii) the binding and cleavage assay of Example 2, allowed for the interrogation of quality and efficacy of neurotoxin preparations in a domain specific manner. Reduced receptor affinity of either mrBoNT/A (FIG. 16A) or mrBoNT/AB (FIG. 16B) was readily detected using a receptor capture sandwich ELISA format. Where evidence of comparable endopeptidase activity to a control (e.g. reference standard) was present, loss of potency observed in the binding and cleavage assay (confirmed by traditional cell based assays) was attributed to topological changes or post translational covalent modification of the neurotoxin heavy chain which binds to its relevant receptor (FIG. 16B). Conversely, in the presence of comparable heavy chain receptor affinity, and reduced endopeptidase activity, (FIG. 16A), loss of potency detected by the binding and cleavage assay, confirmed by traditional cell based assay, can be attributed to loss of endopeptidase kinetic activity.

### Conclusions

In traditional neurotoxin analytical techniques focusing on the potency of preparations, such as the mouse LD₅₀ assay, and more recently cell based assays, the specific reason for changes in potency cannot easily be determined. The well published and well characterised domains of neurotoxins, which each have a specific function in the mode of action, are all essential in producing the extremely high potency and clinical efficacy of clostridial neurotoxin products. Reduced affinity to the relevant neurotoxin receptor will result in reduced cell binding, and therefore cellular penetration, preventing a therapeutic response. Conversely, reduced catalytic activity of neurotoxin preparations will result in slower, or absent SNARE protein cleavage within the cellular cytoplasm. In either event, mouse LD₅₀ or cell-based assays are unable to diagnose a cause of reduced or increased potency.

The cell-free binding and cleavage assay is capable of predicting physiological potency by interrogating the major functions of neurotoxin domains and is comparable to cell based assays in determining potency. Additionally, by combining this with a heavy chain receptor affinity ELISA, and in vitro endopeptidase assay, reduced or increased activity can be readily detected. Therefore, unlike cell based assays, changes in potency can be attributed to specific functions of neurotoxin subunits.

### Example 4

### Materials & Methods

To test binding to SYT-I receptor substrates, mrBoNT/AB (SEQ ID NO: 14 converted into a di-chain toxin by incubation with Lys-C) or wild type BoNT/B (purchased from Metabiologics, Madison, USA) were assessed via bi-layer interferometry (BLI) using the Octet Red 96e system. SYT-I receptor substrates of both mouse (SEQ ID NO: 84) and human sequence (SEQ ID NO: 81) were immobilised on His or GST tag-capture biosensor probes and exposed to a dilution series of mrBoNT/AB (starting at concentrations of 300 μg/ml with a half dilution series) or wild type BoNT/B (starting at concentrations of 500 μg/ml). All data presented represents an average of 3n, where the data has been fitted with a 1:1 binding model within the Octet software, with an acceptance criteria of >0.95 R2 goodness of fit (except for the GT1b assay where data showed a lower R2 goodness of fit).

Single ganglioside preparations of GT1b were purchased from Biosynth UK (catalogue number OO16323). Where GT1b was used, a fixed concentration of 50 μg/ml (24.1 μM) was employed and blanked subtracted against an assay buffer only control. GT1b was introduced into the procedure in both the pre-association baseline step as well as both the association/dissociation phases. This ensured that the receptor-ganglioside complex was formed before toxin association and that the signal would return to its receptor-ganglioside complex baseline during the dissociation phase.

### Results

The table below shows that mrBoNT/AB was able to bind to human and mouse SYT-I. The affinity of mrBoNT/AB to human and mouse SYT-I was further increased by combined use of GT1b. This appeared to arise due to a slower Kdis rate from the SYT-I-GT1b complex compared to SYT-I alone.

mrBoNT/AB (K_{D} in nM)

| Capture substrate | Receptor only | +50 μg/ml GT1b |
|---|---|---|
| SYT-I - Human | 69.0 | 34.9 |
| SYT-I - Mouse | 51.8 | 32.4 |

The table below shows that BoNT/B was able to bind to human and mouse SYT-I.

| Capture substrate | BoNT/B (K_{D} in nM) |
|---|---|
| SYT-I - Human | 1202.0 |
| SYT-I - Mouse | 435.7 |

Thus, SYT-I has been shown to be a suitable capture substrate, optionally together with GT1b, for use in the present invention.

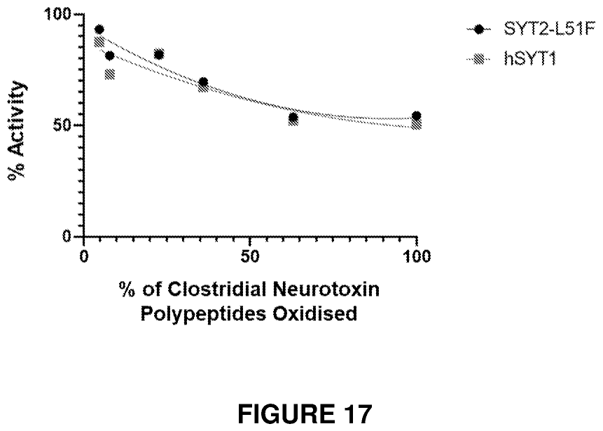

By way of further confirmation, mrBoNT/AB was subjected to forced oxidation and tested in a modified binding and cleavage assay (see Example 2) employing either human SYT-I or human SYT-II (L51F modified) capture substrates. Briefly, mrBoNT/AB was spiked with 0.05% H₂O₂ and held at room temperature, foil wrapped for various time points up to 96 hours, before being quenched with methionine to destroy any remaining peroxide. The % oxidation at various time points was then checked by mass spectrometry. Activity was tested at each timepoint relative to a control/T=0 timepoint. FIG. 17 shows that the assays carried out with the two capture substrates gave very similar activity results, with activity falling as the % of clostridial neurotoxin polypeptides in the composition oxidised at a reference amino acid residue increased.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A cell-free method for determining the clostridial neurotoxin activity of a composition comprising clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding a reducing agent for dissociating light-chain (L-chain) polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining an amount of cleavable substrates cleaved in the assay sample by the L-chain polypeptides, thereby determining the clostridial neurotoxin activity of the composition.

2. The method according to claim 1, wherein the method comprises:
(a) providing capture substrates for the clostridial neurotoxin polypeptides, wherein the capture substrates comprise a clostridial neurotoxin receptor polypeptide or a ganglioside to which the clostridial neurotoxin binds;
(b) contacting the capture substrates with the composition for binding of the clostridial neurotoxin polypeptides to the capture substrates;
(c) removing unbound clostridial neurotoxin polypeptides;
(d) adding a reducing agent for dissociating L-chain polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining an amount of cleavable substrates cleaved in the assay sample by the L-chain polypeptides, thereby determining the clostridial neurotoxin activity of the composition, wherein each cleavable substrate comprises a single-chain polypeptide comprising: (i) a first luciferase domain; (ii) a linker comprising a clostridial neurotoxin cleavage site; and (iii) a second luciferase domain;
wherein the linker functionally joins the first and second luciferase domains, thereby providing a functional luciferase.

3. A cell-free method for determining the clostridial neurotoxin activity of a composition comprising clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing unbound clostridial neurotoxin polypeptides;
(d) adding a reducing agent for dissociating light-chain (L-chain) polypeptides of the bound clostridial neurotoxin polypeptides; and
(e) determining an amount of cleavable substrates cleaved by the L-chain polypeptides, thereby determining the clostridial neurotoxin activity of the composition.

4. The method according to claim 3, wherein the method comprises:
(a) providing capture substrates for the clostridial neurotoxin polypeptides, wherein the capture substrates comprise a clostridial neurotoxin receptor polypeptide or a ganglioside to which the clostridial neurotoxin binds;
(b) contacting the capture substrates with the composition for binding of the clostridial neurotoxin polypeptides to the capture substrates;
(c) removing unbound clostridial neurotoxin polypeptides;
(d) adding a reducing agent for dissociating L-chain polypeptides of the bound clostridial neurotoxin polypeptides; and
(e) determining an amount of cleavable substrates cleaved by the L-chain polypeptides, thereby determining the clostridial neurotoxin activity of the composition, wherein each cleavable substrate comprises a single-chain polypeptide comprising: (i) a first luciferase domain; (ii) a linker comprising a clostridial neurotoxin cleavage site; and (iii) a second luciferase domain;
wherein the linker functionally joins the first and second luciferase domains, thereby providing a functional luciferase.

5. A cell-free method for determining whether or not a composition comprises clostridial neurotoxin polypeptides, the method comprising:
(a) providing capture substrates for the clostridial neurotoxin polypeptides;
(b) contacting the capture substrates with the composition for binding of the clostridial neurotoxin polypeptides to the capture substrates;
(c) removing any unbound clostridial neurotoxin polypeptides;
(d) adding a reducing agent for dissociating any light-chain (L-chain) polypeptides of any bound clostridial neurotoxin polypeptides; and
(e) determining that cleavable substrates have been cleaved, thereby determining that the composition comprises clostridial neurotoxin polypeptides, or determining that cleavable substrates have not been cleaved, thereby determining that the composition does not comprise clostridial neurotoxin polypeptides.

6. The method according to claim 5, wherein the method comprises:
(a) providing capture substrates for the clostridial neurotoxin polypeptides, wherein the capture substrates comprise a clostridial neurotoxin receptor polypeptide or a ganglioside to which the clostridial neurotoxin binds;
(b) contacting the capture substrates with the composition for binding of the clostridial neurotoxin polypeptides to the capture substrates;
(c) removing any unbound clostridial neurotoxin polypeptides;
(d) adding a reducing agent for dissociating any light-chain (L-chain) polypeptides of any bound clostridial neurotoxin polypeptides; and
(e) determining that cleavable substrates have been cleaved, thereby determining that the composition comprises clostridial neurotoxin polypeptides, or determining that cleavable substrates have not been cleaved, thereby determining that the composition does not comprise clostridial neurotoxin polypeptides, wherein each cleavable substrate comprises a single-chain polypeptide comprising: (i) a first luciferase domain; (ii) a linker comprising a clostridial neurotoxin cleavage site; and (iii) a second luciferase domain;
wherein the linker functionally joins the first and second luciferase domains, thereby providing a functional luciferase.

7. The method according to claim 5, wherein the method comprises:
(a) providing capture substrates for the clostridial neurotoxin polypeptides;
(b) contacting the capture substrates with the composition for binding of the clostridial neurotoxin polypeptides to the capture substrates;
(c) removing any unbound clostridial neurotoxin polypeptides;
(d) adding a reducing agent for dissociating any light-chain (L-chain) polypeptides of any bound clostridial neurotoxin polypeptides, thereby providing an assay sample comprising any dissociated L-chain polypeptides and any complexes comprising a capture substrate and clostridial neurotoxin receptor binding domain (H_{CC} domain, e.g. H_{C} domain); and
(e) determining that cleavable substrates have been cleaved in the assay sample, thereby determining that the composition comprises clostridial neurotoxin polypeptides, or determining that cleavable substrates have not been cleaved in the assay sample, thereby determining that the composition does not comprise clostridial neurotoxin polypeptides.

8. The method according to claim 5 or 7, wherein the method comprises:
(a) providing capture substrates for the clostridial neurotoxin polypeptides, wherein the capture substrates comprise a clostridial neurotoxin receptor polypeptide or a ganglioside to which the clostridial neurotoxin binds;
(b) contacting the capture substrates with the composition for binding of the clostridial neurotoxin polypeptides to the capture substrates;
(c) removing unbound clostridial neurotoxin polypeptides;
(d) adding a reducing agent for dissociating L-chain polypeptides of the bound clostridial neurotoxin polypeptides, thereby providing an assay sample comprising dissociated L-chain polypeptides and complexes comprising a capture substrate and clostridial neurotoxin receptor binding domain (H_{CC} domain, e.g. H_{C} domain); and
(e) determining that cleavable substrates have been cleaved in the assay sample, thereby determining that the composition comprises clostridial neurotoxin polypeptides, wherein each cleavable substrate is a single-chain polypeptide comprising: (i) a first luciferase domain; (ii) a linker comprising a clostridial neurotoxin cleavage site; and (iii) a second luciferase domain; wherein the linker functionally joins the first and second luciferase domains, thereby providing a functional luciferase.

9. The method according to any one of the preceding claims, wherein the cleavable substrates are added simultaneously with the reducing agent.

10. The method according to any one of the preceding claims, wherein the cleavable substrate comprises (preferably is) a single-chain polypeptide comprising: (i) a first luciferase domain; (ii) a linker comprising a clostridial neurotoxin cleavage site; and (iii) a second luciferase domain; wherein the linker functionally joins the first and second luciferase domains, thereby providing a functional luciferase, preferably wherein:
(i) when the linker is cleaved, the construct is incapable of exhibiting luciferase activity; and
(ii) when the linker is intact, the construct is capable of exhibiting luciferase activity.

11. A cell-free method for determining whether or not clostridial neurotoxin polypeptides or a portion thereof (e.g. an H-chain, such as an H_{C} or H_{CC} domain thereof) comprised in a composition comprise an activity-altering property, the method comprising:
(a) providing capture substrates for the clostridial neurotoxin polypeptides or the portion thereof (e.g. an H-chain, such as an H_{C} or H_{CC} domain thereof);
(b) contacting the capture substrates with the composition for binding of the clostridial neurotoxin polypeptides or the portion thereof (e.g. an H-chain, such as an H_{C} or H_{CC} domain thereof) to the capture substrates;
(c) removing unbound clostridial neurotoxin polypeptides or the portion thereof (e.g. an H-chain, such as an H_{C} or H_{CC} domain thereof);
(d) determining an amount of the clostridial neurotoxin polypeptides or the portion thereof (e.g. an H-chain, such as an H_{C} or H_{CC} domain thereof) bound to the capture substrates;
(e) comparing the amount of bound clostridial neurotoxin polypeptides or the portion thereof (e.g. an H-chain, such as an H_{C} or H_{CC} domain thereof) with a control; and
(f) determining whether or not clostridial neurotoxin polypeptides or the portion thereof (e.g. an H-chain, such as an H_{C} or H_{CC} domain thereof) comprised in the composition comprise the activity-altering property (e.g. the activity-altering property of the H-chain, e.g. H_{C} or H_{CC} domain) based on the comparison.

12. The method according to any one of the preceding claims, wherein the capture substrate is directly or indirectly immobilised on a solid support, preferably a plastic support.

13. The method according to any one of the preceding claims, wherein the method employs the use of a combination of a capture substrate comprising a clostridial neurotoxin receptor polypeptide and a capture substrate comprising a ganglioside.

14. The method according to any one of the preceding claims, wherein the clostridial neurotoxin polypeptides are botulinum neurotoxin (BoNT) polypeptides.

15. The method according to any one of the preceding claims, wherein the capture substrates comprise an extracellular portion of a BoNT receptor polypeptide, e.g. an extracellular portion of a neuronal BoNT receptor polypeptide.

16. The method according to claim 15, wherein the extracellular portion of the neuronal BoNT receptor polypeptide comprises an amino acid modification and/or a post-translational modification.

17. The method according to claim 15 or 16, wherein the extracellular portion of the BoNT receptor polypeptide is an extracellular portion of synaptotagmin II (SYTII).

18. The method according to claim 17, wherein the extracellular portion of SYTII comprises amino acid residues 1-61 of SYTII.

19. The method according to any one of claims 15-18, wherein the extracellular portion of the BoNT receptor polypeptide is an extracellular portion of human SYTII comprising an L51F substitution.

20. The method according to claim 15 or 16, wherein the extracellular portion of the BoNT receptor polypeptide is an extracellular portion of synaptotagmin I (SYT-I).

21. The method according to any one of claims 14-20, wherein the BoNT polypeptides each comprise a BoNT/B H_{CC} domain.

22. The method according to claim 16, wherein the post-translational modification is a glycosylation, preferably an N-linked glycosylation.

23. The method according to any one of claim 15-16 or 22, wherein the extracellular portion of the BoNT receptor polypeptide is an extracellular portion of SV2c, preferably wherein the extracellular portion comprises SV2c luminal domain 4 (e.g. amino acid residues 519-563 of SV2c).

24. The method according to claim 23, wherein the extracellular portion comprises amino acid residues 473-567 of SV2c.

25. The method according to any one of claim 1-16, 23 or 24, wherein the extracellular portion of the neuronal BoNT receptor polypeptide is an extracellular portion of SV2c comprising a glycosylation at N559.

26. The method according to any one of claims 22-25, wherein the glycosylation comprises a Man-5 glycan, a G0f glycan, a G1f glycan, or a G2f glycan, preferably wherein the glycan further comprises N-acetyl glucosamine (GlcNAc), e.g. G0f-GlcNAc.

27. The method according to any one of the preceding claims, wherein the capture substrate has been produced recombinantly in a mammalian cell, preferably in a human cell.

28. The method according to any one of claim 14-16 or 22-27, wherein the BoNT polypeptides each comprise a BoNT/A H_{CC} domain.

29. The method according to any one of the preceding claims, wherein the method does not comprise the use of a ganglioside, preferably does not comprise the use of GT1b.

30. The method according to any one of the preceding claims, wherein the composition is a first clostridial neurotoxin formulation comprising one or more pharmaceutically acceptable carrier(s), excipient(s), adjuvant(s), propellant(s), and/or salt(s).

31. The method according to claim 30, wherein the clostridial neurotoxin activity of at least a second clostridial neurotoxin formulation is determined using the method according to any one of the preceding claims, wherein the at least second clostridial neurotoxin formulation comprises the same clostridial neurotoxin present in the same amount as the first clostridial neurotoxin formulation and one or more pharmaceutically acceptable carrier(s), excipient(s), adjuvant(s), propellant(s), and/or salt(s), wherein the one or more pharmaceutically acceptable carrier(s), excipient(s), adjuvant(s), propellant(s), and/or salt(s) are different to the one or more pharmaceutically acceptable carrier(s), excipient(s), adjuvant(s), propellant(s), and/or salt(s) present in the first clostridial neurotoxin formulation or are the same but present in a different amount (e.g. a different concentration).

32. The method according to claim 31, wherein the clostridial neurotoxin activity of the first clostridial neurotoxin formulation and the at least second clostridial neurotoxin formulation are compared, and the one or more pharmaceutically acceptable carrier(s), excipient(s), adjuvant(s), propellant(s), and/or salt(s) or amount thereof are selected when the clostridial neurotoxin formulation comprising the same exhibits the highest activity.

33. The method according to any one of claim 1-4, 9-10, or 12-32, further comprising obtaining the results of a heavy-chain binding assay and/or a cell-free substrate cleavage assay.

34. The method according to any one of claim 1-4, 9-10, or 12-33, further comprising comparing the results of a method according to any one of claim 1-4, 9-10, or 12-33 (e.g. the determined clostridial neurotoxin activity of the composition) with the results of a heavy-chain binding assay and/or a cell-free substrate cleavage assay.

35. The method according to claim 34, wherein the comparison allows for the determination as to whether clostridial neurotoxin polypeptides (or portion thereof, e.g. the L-chain or H-chain or portions thereof, such as the H_{C} or H_{CC} domain) comprised in a composition comprise an activity-altering property or as to an amount of clostridial neurotoxin polypeptides (or portion thereof, e.g. the L-chain or H-chain or portions thereof, such as the H_{C} or H_{CC} domain) comprised in a composition that comprise an activity-altering property.

36. Use of an isolated capture substrate for a clostridial neurotoxin for determining the presence or absence of an activity-altering property of clostridial neurotoxin polypeptides or a portion thereof (e.g. an H-chain, such as an H_{C} or H_{CC} domain thereof) comprised in a composition, wherein the isolated capture substrate comprises an extracellular portion of a clostridial neurotoxin receptor polypeptide that comprises an amino acid modification and/or a post-translational modification.

37. The use according to claim 36, wherein the activity-altering property is an activity-reducing H-chain modification, more preferably activity-reducing H-chain oxidation.

38. A method for producing a therapeutic or cosmetic clostridial neurotoxin composition, the method comprising:
(a) obtaining the results of the method of any one of claim 1-4 or 9-10, or 12-35, and formulating and/or packaging the composition for therapeutic or cosmetic use when the clostridial neurotoxin activity is the same as or higher than a positive control (e.g. a positive reference standard); or
(b) subjecting the composition to further purification when the clostridial neurotoxin activity is lower than a positive control (e.g. a positive reference standard), and formulating and/or packaging the further purified composition for therapeutic or cosmetic use.

39. A method for producing a therapeutic or cosmetic clostridial neurotoxin composition, the method comprising:
(a) obtaining the results of a method according to any one of claims 11-35; and
(b) formulating and/or packaging the composition for therapeutic or cosmetic use when the clostridial neurotoxin polypeptides comprised in the composition do not comprise the activity-altering property; or
(c) subjecting the composition to further purification when the clostridial neurotoxin polypeptides comprised in the composition do comprise the activity-altering property; and
(d) formulating and/or packaging the further purified composition for therapeutic or cosmetic use.

40. A therapeutic or cosmetic clostridial neurotoxin composition obtainable by the method according to claim 38 or 39, optionally wherein the therapeutic or cosmetic clostridial neurotoxin composition is packaged.

41. An isolated capture substrate for a clostridial neurotoxin, wherein the capture substrate comprises an extracellular portion of a clostridial neurotoxin receptor polypeptide that comprises an amino acid modification and/or a post-translational modification.

42. A kit comprising:
(a) the isolated capture substrate according to claim 41; and
(b) optionally means for detecting binding of a clostridial neurotoxin (e.g. a botulinum neurotoxin) to the capture substrate; and/or
(c) optionally instructions for the use of the same.


SEQUENCE LISTING (90 sequences)

SEQ ID NO: 1 - protein, 170 aa, synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEIL

SEQ ID NO: 2 - protein, 159 aa, synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 3 - protein, 11 aa, unidentified
VTGYRLFEEIL

SEQ ID NO: 4 - protein, 206 aa, synthetic construct
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 5 - protein, 65 aa, synthetic construct
RENEMDENLEQVSGIIGNLRHMALDMGNEIDTQNRQIDRIMEKADSNKTRIDEANQRATKMLGSG

SEQ ID NO: 6 - protein, 247 aa, synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSSGGGGSRENEMDENLEQVSGIIGNLRHMALDMGNEIDTQNRQIDRIMEKADSNKTRIDEANQRATKMLGSGSGGGGSVTGYRLFEEIL

SEQ ID NO: 7 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 9 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 10 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 11 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 12 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 13 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 14 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 15 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFKKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 16 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLKKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 17 - protein, 1296 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKRKEYRLATNASQAGVEKILSALEIPRVRRLSQVVVMKSKNDQGITNKCKMNLQDRRGNDIGFIGFHQFNNIAKLVASNWYNRQIERRSRRLGCSWEFIPVDDGWGERPL

SEQ ID NO: 18 - protein, 311 aa, synthetic construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSGTENLYFQGMRNIFKRNQEPIVAPATTTATMPIGPVDNSTESGGAGESQEDMFAKLKEKLFNEINKIPLP

SEQ ID NO: 19 - protein, 61 aa, synthetic construct
MRNIFKRNQEPIVAPATTTATMPIGPVDNSTESGGAGESQEDMFAKLKEKLFNEINKIPLP

SEQ ID NO: 20 - protein, 311 aa, synthetic construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSGTENLYFQGMRNIFKRNQEPIVAPATTTATMPIGPVDNSTESGGAGESQEDMFAKLKEKFFNEINKIPLP

SEQ ID NO: 21 - protein, 61 aa, synthetic construct
MRNIFKRNQEPIVAPATTTATMPIGPVDNSTESGGAGESQEDMFAKLKEKFFNEINKIPLP

SEQ ID NO: 22 - protein, 314 aa, synthetic construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSGTENLYFQGMRNIFKRNGEPNVAPATTTATMPLAPVAPADNSTESTGPGESQEDMFAKLKEKFFNEINKIPLP

SEQ ID NO: 23 - protein, 419 aa, Homo sapiens
MRNIFKRNQEPIVAPATTTATMPIGPVDNSTESGGAGESQEDMFAKLKEKLFNEINKIPLPPWALIAIAVVAGLLLLTCCFCICKKCCCKKKKNKKEKGKGMKNAMNMKDMKGGQDDDDAETGLTEGEGEGEEEKEPENLGKLQFSLDYDFQANQLTVGVLQAAELPALDMGGTSDPYVKVFLLPDKKKKYETKVHRKTLNPAFNETFTFKVPYQELGGKTLVMAIYDFDRFSKHDIIGEVKVPMNTVDLGQPIEEWRDLQGGEKEEPEKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSLKPEEEVDALLGKNK

SEQ ID NO: 24 - protein, 422 aa, Homo sapiens
MRNIFKRNQEPNVAPATTTATMPLAPVAPADNSTESTGPGESQEDMFAKLKEKFFNEINKIPLPPWALIAMAVVAGLLLLTCCFCICKKCCCKKKKNKKEKGKGMKNAMNMKDMKGGQDDDDAETGLTEGEGEGEEEKEPENLGKLQFSLDYDFQANQLTVGVLQAAELPALDMGGTSDPYVKVFLLPDKKKKYETKVHRKTLNPAFNETFTFKVPYQELAGKTLVMAIYDFDRFSKHDIIGEVKVPMNTVDLGQPIEEWRDLQGGEKEEPEKLGDICTSLRYVPTAGKLTVCILEAKNLKKMDVGGLSDPYVKIHLMQNGKRLKKKKTTVKKKTLNPYFNESFSFEIPFEQIQKVQVVVTVLDYDKLGKNEAIGKIFVGSNATGTELRHWSDMLANPRRPIAQWHSLKPEEEVDALLGKNK

SEQ ID NO: 25 - protein, 742 aa, Homo sapiens
MEEGFRDRAAFIRGAKDIAKEVKKHAAKKVVKGLDRVQDEYSRRSYSRFEEEDDDDDFPAPSDGYYRGEGTQDEEEGGASSDATEGHDEDDEIYEGEYQGIPRAESGGKGERMADGAPLAGVRGGLSDGEGPPGGRGEAQRRKEREELAQQYEAILRECGHGRFQWTLYFVLGLALMADGVEVFVVGFVLPSAEKDMCLSDSNKGMLGLIVYLGMMVGAFLWGGLADRLGRRQCLLISLSVNSVFAFFSSFVQGYGTFLFCRLLSGVGIGGSIPIVFSYFSEFLAQEKRGEHLSWLCMFWMIGGVYAAAMAWAIIPHYGWSFQMGSAYQFHSWRVFVLVCAFPSVFAIGALTTQPESPRFFLENGKHDEAWMVLKQVHDTNMRAKGHPERVFSVTHIKTIHQEDELIEIQSDTGTWYQRWGVRALSLGGQVWGNFLSCFGPEYRRITLMMMGVWFTMSFSYYGLTVWFPDMIRHLQAVDYASRTKVFPGERVEHVTFNFTLENQIHRGGQYFNDKFIGLRLKSVSFEDSLFEECYFEDVTSSNTFFRNCTFINTVFYNTDLFEYKFVNSRLINSTFLHNKEGCPLDVTGTGEGAYMVYFVSFLGTLAVLPGNIVSALLMDKIGRLRMLAGSSVMSCVSCFFLSFGNSESAMIALLCLFGGVSIASWNALDVLTVELYPSDKRTTAFGFLNALCKLAAVLGISIFTSFVGITKAAPILFASAALALGSSLALKLPETRGQVLQ

SEQ ID NO: 26 - protein, 683 aa, Homo sapiens
MDDYKYQDNYGGYAPSDGYYRGNESNPEEDAQSDVTEGHDEEDEIYEGEYQGIPHPDDVKAKQAKMAPSRMDSLRGQTDLMAERLEDEEQLAHQYETIMDECGHGRFQWILFFVLGLALMADGVEVFVVSFALPSAEKDMCLSSSKKGMLGMIVYLGMMAGAFILGGLADKLGRKRVLSMSLAVNASFASLSSFVQGYGAFLFCRLISGIGIGGALPIVFAYFSEFLSREKRGEHLSWLGIFWMTGGLYASAMAWSIIPHYGWGFSMGTNYHFHSWRVFVIVCALPCTVSMVALKFMPESPRFLLEMGKHDEAWMILKQVHDTNMRAKGTPEKVFTVSNIKTPKQMDEFIEIQSSTGTWYQRWLVRFKTIFKQVWDNALYCVMGPYRMNTLILAVVWFAMAFSYYGLTVWFPDMIRYFQDEEYKSKMKVFFGEHVYGATINFTMENQIHQHGKLVNDKFTRMYFKHVLFEDTFFDECYFEDVTSTDTYFKNCTIESTIFYNTDLYEHKFINCRFINSTFLEQKEGCHMDLEQDNDFLIYLVSFLGSLSVLPGNIISALLMDRIGRLKMIGGSMLISAVCCFFLFFGNSESAMIGWQCLFCGTSIAAWNALDVITVELYPTNQRATAFGILNGLCKFGAILGNTIFASFVGITKVVPILLAAASLVGGGLIALRLPETREQVLM

SEQ ID NO: 27 - protein, 727 aa, Homo sapiens
MEDSYKDRTSLMKGAKDIAREVKKQTVKKVNQAVDRAQDEYTQRSYSRFQDEEDDDDYYPAGETYNGEANDDEGSSEATEGHDEDDEIYEGEYQGIPSMNQAKDSIVSVGQPKGDEYKDRRELESERRADEEELAQQYELIIQECGHGRFQWALFFVLGMALMADGVEVFVVGFVLPSAETDLCIPNSGSGWLGSIVYLGMMVGAFFWGGLADKVGRKQSLLICMSVNGFFAFLSSFVQGYGFFLFCRLLSGFGIGGAIPTVFSYFAEVLAREKRGEHLSWLCMFWMIGGIYASAMAWAIIPHYGWSFSMGSAYQFHSWRVFVIVCALPCVSSVVALTFMPESPRFLLEVGKHDEAWMILKLIHDTNMRARGQPEKVFTVNKIKTPKQIDELIEIESDTGTWYRRCFVRIRTELYGIWLTFMRCFNYPVRDNTIKLTIVWFTLSFGYYGLSVWFPDVIKPLQSDEYALLTRNVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKTGCQITFDDDYSAYWIYFVNFLGTLAVLPGNIVSALLMDRIGRLTMLGGSMVLSGISCFFLWFGTSESMMIGMLCLYNGLTISAWNSLDVVTVELYPTDRRATGFGFLNALCKAAAVLGNLIFGSLVSITKSIPILLASTVLVCGGLVGLCLPDTRTQVLM

SEQ ID NO: 28 - protein, 422 aa, Homo sapiens
MVSESHHEALAAPPVTTVATVLPSNATEPASPGEGKEDAFSKLKEKFMNELHKIPLPPWALIAIAIVAVLLVLTCCFCICKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 29 - protein, 327 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSSGLEVLFQGPVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKT

SEQ ID NO: 30 - protein, 95 aa, synthetic construct
VERDKYANFTINFTMENQIHTGMEYDNGRFIGVKFKSVTFKDSVFKSCTFEDVTSVNTYFKNCTFIDTVFDNTDFEPYKFIDSEFKNCSFFHNKT

SEQ ID NO: 31 - protein, 118 aa, Homo sapiens
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVIYFFT

SEQ ID NO: 32 - protein, 116 aa, Homo sapiens
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 33 - protein, 100 aa, Homo sapiens
MSTGPTAATGSNRRLQQTQNQVDEVVDIMRVNVDKVLERDQKLSELDDRADALQAGASQFETSAAKLKRKYWWKNCKMWAIGITVLVIFIIIIIVWVVSS

SEQ ID NO: 34 - protein, 141 aa, Homo sapiens
MPPKFKRHLNDDDVTGSVKSERRNLLEDDSDEEEDFFLRGPSGPRFGPRNDKIKHVQNQVDEVIDVMQENITKVIERGERLDELQDKSESLSDNATAFSNRSKQLRRQMWWRGCKIKAIMALVAAILLLVIIILIVMKYRT

SEQ ID NO: 35 - protein, 116 aa, Homo sapiens
MAGIELERCQQQANEVTEIMRNNFGKVLERGVKLAELQQRSDQLLDMSSTFNKTTQNLAQKKCWENIRYRICVGLVVVGVLLIILIVLLVVFLPQSSDSSSAPRTQDAGIASGPGN

SEQ ID NO: 36 - protein, 198 aa, Homo sapiens
MKLYSLSVLYKGEAKVVLLKAAYDVSSFSFFQRSSVQEFMTFTSQLIVERSSKGTRASVKEQDYLCHVYVRNDSLAGVVIADNEYPSRVAFTLLEKVLDEFSKQVDRIDWPVGSPATIHYPALDGHLSRYQNPREADPMTKVQAELDETKIILHNTMESLLERGEKLDDLVSKSEVLGTQSKAFYKTARKQNSCCAIM

SEQ ID NO: 37 - protein, 288 aa, Homo sapiens
MKDRTQELRTAKDSDDDDDVAVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 38 - protein, 288 aa, Homo sapiens
MKDRTQELRSAKDSDDEEEVVHVDRDHFMDEFFEQVEEIRGCIEKLSEDVEQVKKQHSAILAAPNPDEKTKQELEDLTADIKKTANKVRSKLKAIEQSIEQEEGLNRSSADLRIRKTQHSTLSRKFVEVMTEYNATQSKYRDRCKDRIQRQLEITGRTTTNEELEDMLESGKLAIFTDDIKMDSQMTKQALNEIETRHNEIIKLETSIRELHDMFVDMAMLVESQGEMIDRIEYNVEHSVDYVERAVSDTKKAVKYQSKARRKKIMIIICCVVLGVVLASSIGGTLGL

SEQ ID NO: 39 - protein, 6 aa, synthetic construct
SGGGGS

SEQ ID NO: 40 - protein, 350 aa, synthetic construct
MGWSCIILFLVATATGVHSGGGGSSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLSSGLEVLFQGPVERDKYANFTINFTMENQIHTGMEYDNGRFIGVKFKSVTFKDSVFKSCTFEDVTSVNTYFKNCTFIDTVFDNTDFEPYKFIDSEFKNCSFFHNKT

SEQ ID NO: 41 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 42 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 43 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 44 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 45 - protein, 1297 aa, Clostridium botulinum
MPVNIKXFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 46 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 47 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 48 - protein, 12 aa, synthetic construct
MHHHHHHDDDDK

SEQ ID NO: 49 - DNA, 3888 bp, synthetic construct
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 50 - DNA, 183 bp, synthetic construct
  source 1..183: mol_type = other DNA
atgcgtaacatcttcaaacgtaaccaagagccgattgttgcgccggcgaccaccaccgcgaccatgccgattggtccggttgacaacagcaccgaaagcggtggcgcgggtgaaagccaagaagatatgtttgcgaagctgaaagagaagttctttaacgaaatcaacaagattccgctgccg

SEQ ID NO: 51 - DNA, 1050 bp, synthetic construct
  source 1..1050: mol_type = other DNA
atgggctggagctgcattattctgtttctggtggcgaccgcgaccggcgtgcatagcggcggcggcggcagcagcccgattctgggctattggaaaattaaaggcctggtgcagccgacccgcctgctgctggaatatctggaagaaaaatatgaagaacatctgtatgaacgcgatgaaggcgataaatggcgcaacaaaaaatttgaactgggcctggaatttccgaacctgccgtattatattgatggcgatgtgaaactgacccagagcatggcgattattcgctatattgcggataaacataacatgctgggcggctgcccgaaagaacgcgcggaaattagcatgctggaaggcgcggtgctggatattcgctatggcgtgagccgcattgcgtatagcaaagattttgaaaccctgaaagtggattttctgagcaaactgccggaaatgctgaaaatgtttgaagatcgcctgtgccataaaacctatctgaacggcgatcatgtgacccatccggattttatgctgtatgatgcgctggatgtggtgctgtatatggatccgatgtgcctggatgcgtttccgaaactggtgtgctttaaaaaacgcattgaagcgattccgcagattgataaatatctgaaaagcagcaaatatattgcgtggccgctgcagggctggcaggcgacctttggcggcggcgatcatccgccgaaaagcgatctgagcagcggcctggaagtgctgtttcagggcccggtggaacgcgataaatatgcgaactttaccattaactttaccatggaaaaccagattcataccggcatggaatatgataacggccgctttattggcgtgaaatttaaaagcgtgacctttaaagatagcgtgtttaaaagctgcacctttgaagatgtgaccagcgtgaacacctattttaaaaactgcacctttattgataccgtgtttgataacaccgattttgaaccgtataaatttattgatagcgaatttaaaaactgcagcttttttcataacaaaacc

SEQ ID NO: 52 - protein, 14 aa, Clostridium botulinum
CLRLTKNSRDDSTC

SEQ ID NO: 53 - protein, 14 aa, Clostridium botulinum
CLRLTRNSRDDSTC

SEQ ID NO: 54 - protein, 17 aa, Clostridium botulinum
CHKAIDGRSLYNKTLDC

SEQ ID NO: 55 - protein, 25 aa, Clostridium botulinum
CVRGIITSKTKSLDEGYNKALNELC

SEQ ID NO: 56 - protein, 25 aa, Clostridium botulinum
CVRGIITSKTKSLDEGYNKALNDLC

SEQ ID NO: 57 - protein, 25 aa, Clostridium botulinum
CVRGIITSKTKSLDKGYNKALNDLC

SEQ ID NO: 58 - protein, 25 aa, Clostridium botulinum
CVRGIIPFKTKSLDEGYNKALNYLC

SEQ ID NO: 59 - protein, 25 aa, Clostridium botulinum
CVRGIIPFKTKSLDEGYNKALNDLC

SEQ ID NO: 60 - protein, 11 aa, Clostridium botulinum
CSNSNTKNSLC

SEQ ID NO: 61 - protein, 15 aa, Clostridium botulinum
CKNIVSVKGIRKSIC

SEQ ID NO: 62 - protein, 15 aa, Clostridium botulinum
CTNIFSPKGIRKSIC

SEQ ID NO: 63 - protein, 15 aa, Clostridium botulinum
CKNIVFSKGITKSIC

SEQ ID NO: 64 - protein, 15 aa, Clostridium botulinum
CKNIVFSKGIRKSIC

SEQ ID NO: 65 - protein, 15 aa, Clostridium botulinum
CKSIVSKKGTKNSLC

SEQ ID NO: 66 - protein, 15 aa, Clostridium botulinum
CLNSSFKKNTKKPLC

SEQ ID NO: 67 - protein, 17 aa, Clostridium botulinum
CKSVIPRKGTKAPPRLC

SEQ ID NO: 68 - protein, 17 aa, Clostridium botulinum
CKSIIPRKGTKAPPRLC

SEQ ID NO: 69 - protein, 17 aa, Clostridium botulinum
CKSIIPRKGTKQSPSLC

SEQ ID NO: 70 - protein, 29 aa, Clostridium tetani
CKKIIPPTNIRENLYNRTASLTDLGGELC

SEQ ID NO: 71 - protein, 15 aa, Clostridium botulinum
CKPVMYKNTGKSEQC

SEQ ID NO: 72 - protein, 10 aa, Clostridium botulinum
CKSVKVPGIC

SEQ ID NO: 73 - protein, 10 aa, Clostridium botulinum
CKSVRAPGIC

SEQ ID NO: 74 - protein, 10 aa, Clostridium botulinum
CKSVKAPGIC

SEQ ID NO: 75 - protein, 10 aa, synthetic construct
TKSLDKGYNK

SEQ ID NO: 76 - protein, 8 aa, synthetic construct
SLDKGYNK

SEQ ID NO: 77 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 78 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 79 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 80 - protein, 60 aa, synthetic construct
MVSESHHEALAAPPVTTVATVLPSNATEPASPGEGKEDAFSKLKEKFMNELHKIPLPPWA

SEQ ID NO: 81 - protein, 310 aa, synthetic construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSGTENLYFQGMVSESHHEALAAPPVTTVATVLPSNATEPASPGEGKEDAFSKLKEKFMNELHKIPLPPWA

SEQ ID NO: 82 - protein, 421 aa, Mus musculus
MVSASRPEALAAPVTTVATLVPHNATEPASPGEGKEDAFSKLKQKFMNELHKIPLPPWALIAIAIVAVLLVVTCCFCVCKKCLFKKKNKKKGKEKGGKNAINMKDVKDLGKTMKDQALKDDDAETGLTDGEEKEEPKEEEKLGKLQYSLDYDFQNNQLLVGIIQAAELPALDMGGTSDPYVKVFLLPDKKKKFETKVHRKTLNPVFNEQFTFKVPYSELGGKTLVMAVYDFDRFSKHDIIGEFKVPMNTVDFGHVTEEWRDLQSAEKEEQEKLGDICFSLRYVPTAGKLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTGAELRHWSDMLANPRRPIAQWHTLQVEEEVDAMLAVKK

SEQ ID NO: 83 - protein, 59 aa, synthetic construct
MVSASRPEALAAPVTTVATLVPHNATEPASPGEGKEDAFSKLKQKFMNELHKIPLPPWA

SEQ ID NO: 84 - protein, 309 aa, synthetic construct
MSGSHHHHHHSSGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLGHTGHRSGTENLYFQGMVSASRPEALAAPVTTVATLVPHNATEPASPGEGKEDAFSKLKQKFMNELHKIPLPPWA

SEQ ID NO: 85 - protein, 6 aa, Clostridium botulinum
NIINTS

SEQ ID NO: 86 - protein, 6 aa, Clostridium botulinum
NIVNTS

SEQ ID NO: 87 - protein, 6 aa, Clostridium botulinum
NITNAS

SEQ ID NO: 88 - protein, 6 aa, Clostridium botulinum
NITNTS

SEQ ID NO: 89 - protein, 6 aa, Clostridium botulinum
EILNNI

SEQ ID NO: 90 - protein, 6 aa, Clostridium botulinum
DILNNI